(19) United States
(12) Patent Application Publication
(10) Pub. No.: US 2007/0297980 A1
(43) Pub. Date: Dec. 27, 2007

(54) Geldanamycin and Derivatives Inhibit Cancer Invasion and Identify Novel Targets

(76) Inventors: Qian Xie, Grand Rapids, MI (US); David Wenkert, Okemos, MI (US); Yuchai Shen, East Lansing, MI (US); George F. Vande Woude, Ada, MI (US); Ricky Hay, Ada, MI (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 10/594,136

(22) PCT Filed: Mar. 28, 2005
(86) PCT No.: PCT/US05/10351

§ 371 Date: Sep. 25, 2006

Related U.S. Application Data
(60) Provisional application No. 60/556,474, filed on Mar. 26, 2004.

(51) Int. Cl. A61K 31/395; A61K 31/397; A61K 51/04; A61P 35/00; A61P 35/04; C07D 225/06

(52) U.S. Cl. 424/1.89; 424/1.85; 514/183; 514/210.21; 540/461

(57) ABSTRACT

Geldanamycin derivatives that block the uPA-plasmin network and inhibit growth and invasion by glioblastoma cells and other tumors at femtomolar concentrations are potentially highly active anti-cancer drugs. GA and various 17-amino-17-demethoxygelddanamycin derivatives are disclosed that block HGF/SF-mediated Met tyrosine kinase receptor-dependent uPA activation at fM levels. Other ansamycins (macbecins I and II), GA derivatives, and radicicol required concentrations several logs higher (≧nM) to achieve such inhibition. The inhibitory activity of tested compounds was discordant with the known ability of drugs of this class to bind to hsp90, indicating the existence of a novel target(s) for HGF/SF-mediated events in tumor development. Methods of using such compounds to inhibit cancer cell activities and to treat tumors are disclosed. Such treatment with low doses of these highly active compounds provide an option for treating various Met-expressing tumors, in particular invasive brain cancers, either alone or in combination with conventional surgery, chemotherapy, or radiotherapy.

### BACKGROUND OF THE INVENTION

1. Field of the Invention

The present invention in the field of cancer pharmacology is directed to chemical derivatives of geldanamycin (1), some of which are novel compounds, that inhibit cancer cell activities at femtomolar concentrations, and the use of these compounds to inhibit HGF-dependent, Met-mediated tumor cell activation, growth, invasion, and metastasis. These compounds, acting on a novel, yet unidentified target, are exquisitely potent anticancer agents.

2. Description of the Background Art

Geldanamycin (GA) is an ansamycin natural product drug (Sasaki K et al, 1970; DeBoer C et al, 1970). Geldanamycins (GAs) are referred to here as a class of GA derivatives some of which demonstrated anti-tumor activity in mouse xenograft models of human breast cancer, melanoma, and ovarian cancer (Schulte T W et al, 1998; Webb C P et al., 2000). Moreover, drugs of the GA class reduced the expression of several tyrosine kinase and serine kinase oncogene products, including Her2, Met, Raf, cdk4, and Akt (Blagosklonny, 2002; Ochel et al., 2001; Schulte et al, supra); Solit et al., 2002; Webb et al., supra. These drugs have been found to act at concentrations in the nanomolar range (and are thus referred to herein as nM GA inhibitors or “nM-GAi”) by inhibiting the molecular chaperone HSP90, thereby preventing proper folding of client oncoproteins, leading to their destabilization (Bonvini et al., 2001; Ochel et al., 2001). Moreover, some of the compounds drugs listed in Webb et al. (supra) as supplied by the National Cancer Institute Anticancer Drug Screen NCI-Ads were found to be impure (by thin layer chromatography), leading to a conclusion that earlier results and interpretations may likely be incorrect.

Recent work has shown that the Met signaling pathway is a potential therapeutic target for cancer therapy. Met-directed ribozyme and anti-sense strategies reduced Met and HGF/SF expression, tumor growth and metastatic tumor potential (Abounader, R et al., 1999; Jiang, W G et al., 2001; Abounader, R et al., 2002; Stabile, L P et al., 2004). NK4, a HGF/SF fragment possessing its N-terminal four-kringle domain, is a competitive HGF/SF antagonist for the Met receptor (Date, K et al., 1997) and has been demonstrated to inhibit tumor invasion and metastasis, as well as tumor angiogenesis (Matsumoto, K et al., 2003). Monoclonal antibodies directed to HGF/SF neutralizes its activity with inhibition of human xenograft tumor growth in athymic nu/nu mice (Cao, B et al., 2001). The indole-based receptor tyrosine kinase inhibitors K252a and PHA-665752 inhibit Met kinase activity and Met-driven tumor growth and metastatic potential (Morotti, A et al., 2002; Christensen, J G et al., 2003).

Webb et al. (2000) screened inhibitors of the Met receptor signal transduction pathway that might inhibit tumor cell invasion. HGF/SF induces the expression of the urokinase plasminogen activator (uPA) and its receptor (uPAR), mediators of cell invasion and metastasis. Webb et al. (2000) described a cell-based assay utilizing the induction of uPA and uPAR and the subsequent conversion of plasminogen to plasmin which allowed the screening of compounds for inhibitory properties in MDCK-2 cells. Geldanamycin (1) and some derivatives thereof were found to exhibit high inhibitory activity: at femtomolar (fM) concentrations. This exquisite inhibitory activity has been by the present inventors (as disclosed below) to include additional activities of the invasion complex, notably the in, vitro invasion of human tumor cells through three-dimensional Matrigel®. No loss of Met expression was observed at lower than nanomolar (nM) concentrations, indicating that the observed inhibitory activity was independent of down-regulation of the Met receptor.

Geldanamycin and 17-alkylamino-17-demethoxygeldanamycin derivatives are best known for their ability to bind to the ATP binding site of the amino-terminal domain region of heat shock protein 90 (hsp90) (Stebbins, C E et al., 1997; Grenert, J P et al., 1997; Schulte, T W et al., 1998; Roe, S M et al., 1999; Jez, J M et al., 2003). Hsp90 belongs to the structural protein family of GHKL ATPases (Dutta, R et al., 2000). This abundant protein helps regulate activity, turnover, and trafficking of various critical proteins. It facilitates folding and regulation of proteins in cellular signaling, such as transcription factors, steroid receptors, and protein kinases (Fink, A L, 1999; Richter, I et al., 2001; Picard, D, 2002; Pratt, W B et al., 2003). The function of hsp90 is blocked by ansamycin natural products, such as GA and macbecin I (2) (Blagosklonny M V et al., 1996; Bohen S P et al., 1998), as well as radicicol (3) (Whitesell, L et al., 1994; Sharma, S V et al., 1998; Schulte, T W et al., 1998) (see Description of Invention for chemical structures). The antitumor effect of 17-allylamino-17-demethoxygeldanamycin (4), a drug now in clinical trials, has been attributed to the blockage of hsp90 function (Maloney A et al., 2002; Neckers, L et al., 2003).

A drawback to the clinical use of GA are its solubility and toxicity limitations, but the derivative 17-allylamino-17-demethoxygeldanamycin (abbreviated 17-AAG) (4) (also designated NSC.330507), had tumor inhibitory activity with lower toxicity (Kamal A et al., 2003 Nature 425:407-410) and is being evaluated in phase I-II clinical trials (Goetz M P et al., (2003) Annals Oncol. 14: 1169-1176; Maloney T et al., (2001) Expert Opin. Biol. Ther. 2: 3-24). Another GA derivative in preclinical evaluation, which has greater solubility in water and is available for oral delivery, is 17-(dimethylaminoethyl)amino-17-demethoxygeldanamycin (5) (abbreviated 17-DMAG) was essentially 100% when given i.p., about twice that of orally delivered 17-AAG (4) (Egorin M J et al., 2002). 17-amino-17-demethoxygeldanamycin (6, a metabolite of 17-AAG (4), has equivalent biological activity as determined by the ability to decrease p185^{erbB2} and is under development as a potential therapeutic (Egorin M J et al. (1998)). Both GA and 17-AAG can sensitize breast cancer cells to Taxol- and doxorubicin-mediated apoptosis (Munster P N et al., (2001) Clin. Cancer Res. 1: 2228-2236).

U.S. Pat. No. 4,262,989 (to Sasaki et al.) discloses various geldanomycin derivatives substituted at the C17 and C19 position. The substituents at both these positions are listed as including an amine which may be di-substituted with various radicals including alkyl groups (C₂₋₁₂) which may be further substituted with hydroxy, amino, methylamino, pyrrolidino, pyridinyl, methoxy, piperidino, morpholino, halogens, cycloalkyls and other groups. These compounds are said to inhibit growth in vitro of a particular “cancer cell, which is, in effect, a murine fibroblast clone transformed by an oncogenic virus.

Rosen et al., WO98/51702(1998, Nov. 19) disclose GA derivatives coupled to Hsp90-targeting moieties which comprises both a targeting moiety that binds specifically to a protein, receptor or marker and ah hsp09-binding moiety which binds to the hsop90 pocket to which ansamycin antibiotics bind. This document discloses reacting GA with aziridine to produce compound 15 as disclosed herein, which is an intermediate in the synthesis process. This compound is reacted with cyanogen iodide (ICN) to produced 17-(N-iodoethyl-N-cyano-17-demethoxygeldanamycin. The latter analogue bund to HSP90, and was readily radiolabeled during synthesis by using radioactive ICN. It was disclosed that the “corresponding 17-N-iodoakly-N-cyano) compounds can be made using azetidine (3 carbons), pyrrolidine (4 carbons), etc., in place of aziridine.”

Gallaschun et al., WO95/01342 (11995, January 12) disclose various ansamycin derivatives as inhibitors of oncogene products and as antitumor/anticancer agents. See page 15, line 19, through page 17, line 12, and Examples 2-99.

U.S. Pat. No. 5,932,566 to Schnur et al.) disclose a large number of GA derivatives which are substituted at the following ring positions of GA, including C4, 5, 11, 17, 19, and 22. The compound are said to inhibit growth of SKBr3 breast cancer cells in vivo, although no results showing any antitumor effects at any level are provided.

PCT Publication WO 2004/087045 (2004, Oct. 14) discloses GA analogues as preventing or reducing restenosis alone or in combination with other drugs. At page 4, the following compounds are mentioned: 17-Allylamino-17-demethoxygeldanamycin (present compound 4) 7-[2-dimethylamino)ethylamino]-demethoxy-11-O-methylgeldanamycin and 17-N-Azetidinyl-17-demethoxygeldanamycin (present compound 14).

The Met receptor tyrosine kinase and its ligand, hepatocyte growth factor/scatter factor (HGF/SF), contribute to tumorigenesis and metastasis. Inappropriate Met expression is highly correlated with metastasis and reduced overall survival of patients with cancer (Birchmeier et al., 2003; Maulik et al., 2002b), and both Met and HGF/SF have been implicated in many types of human and animal carcinomas and sarcomas. See URL <vai.org/metandcancer/> for an inclusive list, which is incorporated by reference in its entirety. Met signaling induces proliferation and invasion in vitro and tumorigenesis and metastasis in animal models. HGF/SF is a potent angiogenic and survival molecule (Birchmeier et al., supra). One consequence of Met activation by HGF/SF is induction of the urokinase-type plasminogen activator (uPA) proteolysis network, an important factor in tumor invasion and metastasis. Exposure of Met-expressing cells to HGF/SF induces the expression of uPA and/or the uPA receptor (uPAR), leading to plasmin production by cleavage of plasminogen (Hattori et al., 2004; Jeffers et al., 1996; Tacchini et al., 2003). To search for drugs that might inhibit tumor cell invasion, Webb et al. (2000) developed a cell-based assay in canine kidney MDCK epithelial cells and searched for compounds that inhibit uPA activity. Several derivatives of GA inhibited uPA activity at femtomolar (fM) concentrations (fM-GAi), around 6 orders of magnitude below the nM concentrations required to reduce Met expression (Webb et al., 2000). These studies suggested that MDCK cells possess a novel target for fM-GAi drugs that is high in affinity and likely low in abundance.

The target(s) for disruption of the Met signal transduction pathway at fM levels in tumor cells by GA and its derivatives remains unknown. The above described disruption of hsp90 function is an effect of this ansamycin class of drugs known to occur at higher concentrations, i.e., micromolar (ELM) and greater. The present inventors have assessed the structure-activity relationship of GA derivatives for an unknown target(s) and have been able to distinguish the fM target(s) from hsp90.

There is a need in the art for highly potent compounds of the GA class as novel anti-cancer therapeutics that are effective at very low concentrations. The present invention responds to that need.

Previous work from the present inventors' laboratory showed that only 4 out of over 30 GA-derived drugs provided by the NCI Anti-Neoplastic Drug Screen Program (NCI ADS) inhibited the activation of urokinase plasminogen activator (uPA)-plasmin by hepatocyte growth factor/scatter factor (HGF/SF) in MDCK cells at femtomolar concentrations (Ref. 1: Webb C P et al., Cancer Res. 60: 342-3491). There drugs are referred to herein as “fM-GAi” drugs versus drugs of the GA family drugs that show activity in the nanomolar range (referred to as “nM-GAi” drugs.

### SUMMARY OF THE INVENTION

The present inventors have discovered that the femtomolar (or even lower) activity of certain GA derivatives (“fM-Gai” compounds) on inhibiting the uPA proteolysis network in MDCK cells is HGF/SF dependent. Such sensitivity is also present in human tumor cells in which uPA activity can be significantly up-regulated by HGF/SF.

In addition to inhibiting HGF/SF-mediated uPA induction, fM-GAi compounds, including various 17-amino-17-demethoxygeldanamycin derivatives, were found to inhibit HGF/SF-induced scattering of MDCK cells and in vitro invasive activity of several human glioblastoma cell lines—DBTRG, SNB19 and U373. However, it is disclosed herein that HSP90 is not the fM-GAi target. First, not all HSP90-binding compounds display fM-GAi activity. Radicicol (RA), which binds to HSP90 with high affinity (Roe et al., 1999; Schulte et al., 1999) inhibits HGF/SF-induced uPA activation not at concentrations below nM. GA, a fM-GAi drug, other ansamycins including macbecins I and II (MA)), certain GA derivatives, and radicicol inhibit uPA activity and Met expression in parallel at nM concentrations. Using various cell lines and nM concentrations of these agents, the present inventors showed that all available HSP90 binding sites were occupied. However, at GA at picomolar (pM) and lower concentrations, at which HSP90 is unoccupied by GA, and Met protein levels remain unaffected, uPA activity, cell scattering and tumor cell invasion were still inhibited. Thus, fM-GAi drugs are potent inhibitors of important biological activities of HGF/SF such as tumor cell invasion but do not mediate this effect through HSP90. This indicates a novel target(s) for HGF/SF-mediated uPA activation.

Thus, these fM-GAi compounds are drug candidates for interfering with tumor cell invasion, and may be combined with surgery, conventional chemotherapy, or radiotherapy to prevent cancer cell invasion. They also have utility as diagnostic/prognostic agents when coupled with detectable labels such as radionuclides.

Specifically, the present invention is directed to a compound of Formula I or Formula II or a pharmaceutically acceptable salt thereof which has the property of inhibiting the activation of Met by HGF/SF in cancer cells at a concentration below 10⁻¹¹ M, wherein:

R¹ is lower alkyl, lower alkenyl, lower alkynyl, optionally substituted lower alkyl, alkenyl, or alkynyl; lower alkoxy, alkenoxy and alkynoxy; straight or branched alkylamines, alkenyl amines and alkynyl amines; a 3-6 member heterocyclic group that is optionally substituted (and R¹ is preferably a 3-6 member heterocyclic ring wherein N is the heteroatom).

R² is H, lower alkyl, lower alkenyl, lower alkynyl, optionally substituted lower alkyl, alkenyl, or alkynyl; lower alkoxy, alkenoxy and alkynoxy; straight and branched alkylamines, alkenyl amines and alkynyl amines; a 3-6 member heterocyclic group that is optionally substituted;

R³ is H, lower alkyl, lower alkenyl, lower alkynyl, optionally substituted lower alkyl, alkenyl, or alkynyl; lower alkoxy, alkenoxy and alkynoxy; straight or branched alkylamines, alkenyl amines, alkynyl amines; or wherein the N is a member of a heterocycloalkyl, heterocylokenyl or heteroaryl ring that is optionally substituted;

R⁴ is H, lower alkyl, lower alkenyl, lower alkynyl, optionally substituted lower alkyl, alkenyl, or alkynyl, and wherein

the ring double bonds between positions C₂═C₃, C₄═C₅, and C₈═C₉ are optionally hydrogenated to single bonds.

The compound preferably inhibits the activation of Met by HGF/SF in cancer cells at a concentration below 10⁻¹¹ M or below 10⁻¹² M, below 10⁻¹³ M or below 10⁻¹⁴ M or below 10⁻¹⁵ M or below 10⁻¹⁶ M or below 10⁻¹⁷ M or below 10⁻¹⁸ M or below 10⁻¹⁹ M.

In a preferred embodiment, R¹ is a substituent as indicated and each of R², R³ and R⁴ is H.

The compound is preferably selected from the group consisting of:
(a) 17-(2-Fluoroethyl)amino-17-demethoxygeldanamycin;
(b) 17-Allylamino-17-demethoxygeldanamycin;
(c) 17-N-Aziridinyl-17-demethoxygeldanamycin;
(d) 17-Amino-17-demethoxygeldanamycin;
(e) 17-N-Azetidinyl-17-demethoxygeldanamycin;
(f) 17-(2-Dimethylaminoethyl)amino-17-demethoxygeldanamycin;
(g) 17-(2-Chloroethyl)amino-17-demethoxygeldanamycin; and
(h) Dihydrogeldanamycin

Also provided is pharmaceutical composition comprising the above compound and a pharmaceutically acceptable carrier or excipient.

The invention is directed to a method of inhibiting a HGF/SF-induced, Met receptor mediated biological activity of a Met-bearing tumor or cancer cell, comprising providing to said cells an effective amount of a compound as above 9 which compound has an IC₅₀ of less than about 10⁻¹¹ M or less than about 10⁻¹² M or less than about 10⁻¹³ M or less than about 10⁻¹⁴ M or less than about 10⁻¹⁵ M or less than about 10⁻¹⁶ M or less than about 10⁻¹⁷ M or less than about 10⁻¹⁸ M for inhibition of said biological activity. The biological activity may be the induction of uPA activity in the cells, growth in vitro or in vivo, or scatter of the cells, invasion of said cells in vitro or in vivo.

Also included is a method of inhibiting in a subject metastasis of Met-bearing tumor or cancer cells that is induced by HGF/SF, comprising providing to said subject an effective amount of a compound as disclosed herein which compound has an IC₅₀ of less than about 10⁻¹¹ M or lower, as indicated above for inhibition tumor cell invasion when measured in an assay in vitro. Preferably, the inhibition results in measurable regression of a tumor caused by said cells or measurable attenuation of tumor growth in said subject.

A method of protecting against growth or metastasis of a Met-positive tumor in a susceptible subject, preferably a human, comprises administering to said subject who is either

(a) at risk for development of said tumor,

(b) in the case of an already treated subject, at risk for recurrence of said tumor,

an effective amount of the compound as above.

The above compound detectably labeled with a halogen radionuclide preferably bonded to the R¹ group, preferably selected from the group consisting of ¹⁸F, ⁷⁶Br, ⁷⁶Br, ¹²³I, ¹²⁴I, ¹²⁵I, and ¹³¹I.

### BRIEF DESCRIPTION OF THE DRAWINGS

FIGS. 1 and 2. Activity of representative GA derivative compounds. Absorbances were read at 405 nm following initial MDCK cell exposure to HGF/SF in absence or presence of varying concentrations of tested compounds and exposure 24 hours later to a plasmin-sensitive chromophore. Values displayed represent mean values ±1 S.D. from triplicate assays at each concentration of each tested compound.

FIG. 3-6. Effects of GA and related compounds on uPA inhibition in human tumor cell lines. Cells were incubated for 24 hours with 60 units/ml HGF/SF in the absence or presence of various concentrations of GA and related compounds as indicated. The uPA activity assay (upper panels) was performed on MDCK cells essentially as previously described (Webb et al, 2000). Examples. Cells used were as follows: FIG. 3—MDCK; FIG. 4—DBTRG; FIG. 5—U373; FIG. 6—SNB19. Test compounds included RA and MA and were used at the indicated concentrations. GA derivatives are abbreviated as follows: GA=geldanamycin; 17-AAG=17-allylamino-17-demethoxygeldanamycin and 17-ADG=17-amino-17-demethoxygeldanamycin.

FIGS. 7-9. Effects of GA and related compounds on proliferation o human tumor cell lines. Normalized cell growth results from drug treated cells were normalized to the mean value obtained from cells stimulated with HGF/SF in the absence of drug and are expressed as a percentage of control. Values displayed represent mean values ±1 s.d. from triplicate assays (MTS assay described in Examples) at each concentration of each test compound. Cells used were as follows: FIG. 7-BTRG; FIG. 8—U373; FIG. 9—SNB19. Test compounds and abbreviations described for FIGS. 3-6, above.

FIG. 10. Effects of GA on cell scattering. MDCK cells were seeded in 96-well plates at 1500 cells/well in triplicate and HGF/SF (100 ng/ml) was added alone or in the presence of GA 24 hrs later: After an additional 24 hrs the cells were fixed and stained using Diff-Quik stain set. Representative micrographs of treated MDCK cell preparations are shown in the panels as follows: MDCK cells (a-j); HGF/SF treated cells (b-j); plus GA at 10⁻⁷ M in (c); GA at 10⁻⁹ M in (d); GA at 10⁻¹³ M in (e); GA at 10⁻¹⁵ M in (f); 17-AAG at 10⁻⁷ M (g); 17-AAG at 10⁻⁹ M in (h); 17-AAG at 10⁻¹³M in (i); 17 AAG at 10⁻¹⁵ M in (j).

FIGS. 11-13: Effects of GA on cell invasion in vitro. DBTRG (FIG. 11), SNB19 (FIG. 12) and U373 (FIG. 13) cells were measured by the Matrigel invasion assay as described in the Example 19. Cells penetrating the Matrigel® layer were counted after 24 hrs of drug exposure. Each bar represents the mean ±1 s.d. for cell number from triplicate samples.

FIG. 14. Effects of MA and GA exposure on HSP90α and Met expression. MDCK and DBTRG cells were treated with HGF/SF (100 ng/ml) in the presence of mecbecine (MA) or GA at the indicated concentrations. Cell lysates were analyzed as described in Example 10. An aliquot of each cell lysate was also incubated with GA-affinity beads as described in and eluates from the beads were analyzed by SDS-PAGE followed by immunoblotting with antibody against HSP90α. Control cultures received no HGF/SF and no test compound. Relevant regions of the resulting fluorograms are shown: Samples for lanes 1-6 and 7-10 are respectively from MDCK and DBTRG total cell lysates. HSP90α was detected in pull-down experiments with GA gel beads (upper panel) or in whole cell lysates (lower panel) in Western blots with anti-HSP90α antibody. Samples in lanes 2-6 and 8-12 were from cells treated with HGF/SF. Samples in lanes 3, 4 and 9, 10 were from cells treated with MA as indicated. Samples in lanes 5, 6, and 11, 12 were from cells treated with GA as indicated.

FIG. 15. Effects of long-term MDCK cell culture in MA on sensitivity of Met and HSP90α to nM-GAi and fM-GAi drug challenge. MDCK cells were maintained for 2-3 months in MA at concentrations of 1, 2 or 3×10⁻⁶ M to generate MDCKG1, MDCKG2, and MDCKG3 cells, respectively. 10⁶ parental MDCK cells or long-term-exposed cells (G1-G3) were seeded in dishes, grown to 80% confluency, and then further exposed to either GA (+GA, 10⁻⁶ M) or MA (+MA, 10⁻⁵ M) for 24 hours. Cells were harvested, lysed, and lysates analyzed for relative abundance of Met, HSP90α, and β-actin (loading control) by Western blots (see Example 19). Relevant regions of the resulting fluorograms are shown.

FIG. 16. HGF/SF-Met signaling in cell cultures exposed long-term to MA. 2.5×10⁵ cells of parental MDCK cells and MA maintained MDCKG3 cells were seeded in 60×15 mm dishes and exposed to HGF/SF (100 ng/ml) 24 hours later. At the indicated times, cells were harvested, lysed, and lysates were analyzed for relative abundance of total and phosphorylated Met, total and phosphorylated Erk1, Erk2, and β-actin (loading control) by Western blots with appropriate antibodies (see Example 19). Relevant regions depicting Met, p-Met, Erk 1, Erk2, and p-Erk1, p-Erk2 in the resulting fluorograms are shown.

FIG. 17. Effects of MA and GA on HGF/SF stimulated scattering in MDCK AND MDCKG3 cells. 1500 cells of parental MDCK cells (panel a-c) or MDCKG3 cells maintained in 3×10⁻⁶ M MA (panels d-i) were seeded in 96-well plates. HGF/SF was added 24 hrs later alone (HGF/SF, 100 ng/ml), with MA (3×10⁻⁶ M) or with GA (10⁻⁷ to 10⁻¹⁵ M). 24 hrs later, scattering was evaluated microscopically. Representative micrographs (100×) are shown: (a) Control MDCK cells; (b) MDCK cells+HGF/SF; (c) MDCK cells+HGF/SF+MA (3×10⁻⁶ M); (d) Control MDCKG3 cells; (e) MDCKG3 cells+HGF/SF; (f) MDCKG3 cells+HGF/SF plus GA (10⁻⁷ M); (g) MDCKG3 cells+HGF/SF+GA (10⁻⁹ M); (h) MDCKG3 cells+HGF/SF plus GA (10⁻¹³ M); (i) MDCKG3 cells+HGF/SF plus GA (10⁻¹⁵ M).

FIG. 18. Effects of MA and GA on HGF/SF-stimulated uPA induction in MDCK AND MDCKG3 cells. 1500 cells were seeded and treated with HGF/SF or with macbecin II (MA) or geldanamycin (GA). After an additional 24 hours of incubation, cells were washed twice with DMEM, and 200 μl of reaction buffer containing the plasmin-sensitive chromophore was added to each well. The plates were then incubated at 37° C., 5% CO₂ for 4 h, at which time the absorbances generated were read on an automated spectrophotometric plate reader at a single wavelength of 405 nm.

### DESCRIPTION OF THE PREFERRED EMBODIMENTS

Ansamycins, including geldanamycin and the derivative 17-allylamino-17-demethoxygeldanamycin, and radicicol are known for their ability to tightly bind heat shock protein 90, a presumed mechanism for their actions on cells. Indeed GA and 17-alkylamino-17-demethoxygeldanamycin bind to the ATP binding site of the amino-terminal domain hsp90)

The present inventors have discovered that geldanamycin (GA) and some of its derivatives inhibit at femtomolar levels HGF/SF-mediated Met tyrosine kinase receptor activation, which can be measured as receptor-dependent activation of uPA. Assessment is of structural requirements for such activity led to the conclusion that the target of this activity is not HSP90, but rather an unknown protein of complex.

A number of compounds were synthesized (or obtained from the National Cancer Institute) and tested and are discussed below. See Examples 1-19. Compounds 1-3 are GA, macbecin and radicicol respectively.

| | |
|---|---|
| 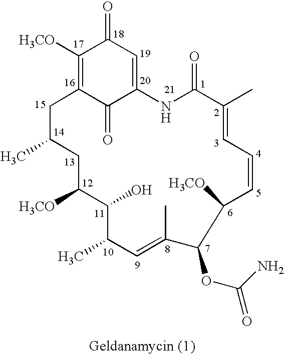 | |
| 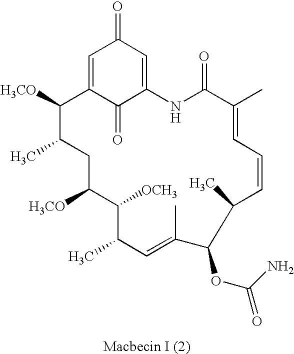 | |
| 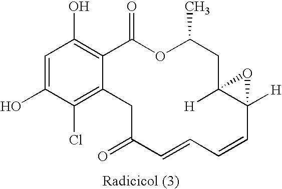 | |
| 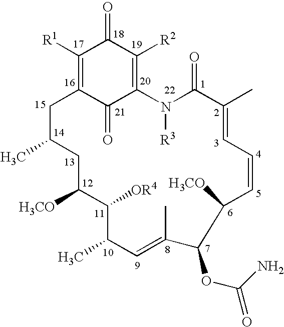 | Formula I |
| 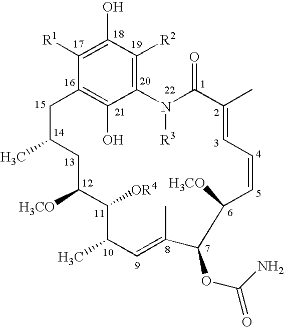 | Formula II |

| Cpd | R¹ | R² | R³ | R⁴ |
|---|---|---|---|---|
| 4 | —NHCH₂CH═CH₂ | H | H | H |
| 5 | —NHCH₂CH₂N(CH₃)₂ | H | H | H |
| 6 | —NH₂ | H | H | H |
| 7 | —NHCH₂CH₂Cl | H | H | H |
| 8 | —NHCH₂CH₂F | H | H | H |
| 9 | —NHCH₂CH₂NHC(O)CH₃ | H | H | H |
| 10 | —NH(CH₂)₆NHC(O)CH₃ | H | H | H |
| 11 | —NH(CH₂)₆NH—biotinyl | H | H | H |
| 12 | —NH(CH₂CH₂O)₂CH₂CH₂NHC(O)CH₃ | H | H | H |
| 13 | —NHCH₂CO₂H | H | H | H |
| 14 | —NCH2CH2CH2-(azetidinyl) | H | H | H |
| 15 | —NCH2CH2-(aziridinyl) | H | H | H |

Two additional structures shown in Formulas III and IV with the indicated substituents were synthesized and studied (see Examples). One of these compounds, 14 also appears above as a substituent of Formulas I or II.

It should be noted that active compounds of the present invention, particularly those with fm-GAi activity, can have either the oxidized (benzoquinone, Formula I) or the reduced (hydroquinone, Formula II) structure.

| | |
|---|---|
| | Formula III |
| 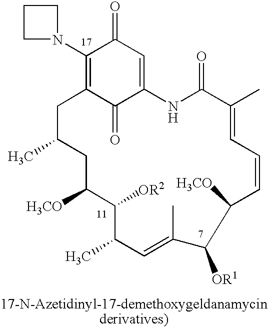 | |

| Cpd | R¹ | R² |
|---|---|---|
| 14 | —C(O)NH₂ | —H |
| 18 | —C(O)NH₂ | —C(O)CH₃ |
| 19 | —H | —H |

| | |
|---|---|
| | Formula IV |
| 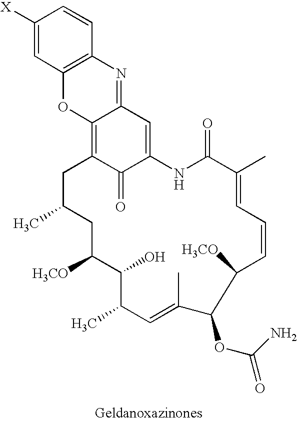 | |

| Cpd | X |
|---|---|
| 16 | Br |
| 17 | I |

Unless indicated otherwise, the alkyl, alkoxy, and alkenyl moieties referred to herein may comprise linear, branched and cyclic moieties and combinations thereof and the term “halo” includes fluoro, chloro, bromo and iodo. It is clear that a group comprising only 1 or 2 atoms cannot be branched or cyclic. Furthermore, unless otherwise indicated “optionally substituted” means comprising from zero to the maximum number of substituents, e.g., 3 for a methyl group, 5 for a phenyl group, etc. As used herein the term “alkyl”, denotes straight chain, branched or cyclic fully saturated hydrocarbon residues. Unless the number of carbon atoms is specified, “alkyl” term refers to C₁₋₆ alkyl groups (also called “lower alkyl”). When “alkyl” groups are used in a generic sense, e.g., “propyl,” “butyl”, “pentyl” and “hexyl,” etc., it will be understood that each term may include all isomeric forms (straight, branched or cyclic) thereof.

A preferred alkyl is C₁₋₆ alkyl, more preferably C₁₋₄ alkyl or C₁₋₃ alkyl. Examples of straight chain and branched alkyl groups are methyl, ethyl, n-propyl, isopropyl, n-butyl, sec-butyl, tert-butyl, n-pentyl, iso-pentyl, 1,2-dimethylpropyl, 1,1-dimethylpropyl.

Example of cycloalkyl groups are cyclopropyl, cyclopropylmethyl, cyclopropylethyl, cyclobutyl, cyclopentyl, cyclohexyl, etc.

An alkyl group, as defined herein, may be optionally substituted by one or more substituents. Suitable substituents may include halo; haloalkyl (e.g., trifluoromethyl, trichloromethyl); hydroxy; mercapto; phenyl; benzyl; amino; alkylamino; dialkylamino; arylamino; heteroarylamino; alkoxy (e.g., methoxy, ethoxy, butoxy, propoxy phenoxy; benzyloxy, etc.); thio; alkylthio (e.g. methyl thio, ethyl thio); acyl, for example acetyl; acyloxy, e.g., acetoxy; carboxy (—CO₂H); carboxyalkyl; carboxyamide (e.g., —CONH-alkyl, —CON(alkyl)₂, etc.); carboxyaryl and carboxyamidoaryl (e.g., CONH-aryl, —CON(aryl)₂); cyano; or keto (where a CH₂ group is replaced by C═O).

As used herein the term “alkenyl” denotes groups formed from straight chain, branched or cyclic hydrocarbon residues containing at least one C═C double bond including ethylenically mono-, di- or poly-unsaturated alkyl or cycloalkyl groups as previously defined. Thus, cycloalkenyls are also intended. Unless the number of carbon atoms is specified, alkenyl preferably refers to C₂₋₈ alkenyl. More preferred are lower alkenyls (C₂₋₆), preferably C₂₋₅, more preferably C₂₋₄ or C₂₋₃. Examples of alkenyl and cycloalkenyl include ethenyl, propenyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1,4-pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl and 1,3,5,7-cyclooctatetraenyl. Preferred alkenyls are straight chain or branched. As defined herein, an alkenyl group may optionally be substituted by the optional substituents described above for substituted alkyls.

As used herein the term “alkynyl” denotes groups formed from straight chain, branched or cyclic hydrocarbon residues containing at least one C≡C triple bond including ethynically mono-, di- or poly-unsaturated alkyl or cycloalkyl groups as previously defined. Unless the number of carbon atoms is specified, the term refers to C₂₋₆ alkynyl (lower alkynyl), preferably C₂₋₅, more preferably C₂₋₄ or C₂₋₃ alkynyl. Examples include ethynyl, 1-propynyl, 2-propynyl, butynyl (including isomers), and pentynyl (including isomers). Preferred alkynyls are straight chain or branched alkynyls. As defined herein, an alkynyl may optionally be substituted by the optional substituents described above for alkyl.

The terms “alkoxy” refer to alkyl groups respectively when linked by oxygen. GA (1) has a methoxy group (—OCH₃) substituting the 17 C position (i.e., R¹ of Formula I is —CH₃). Other groups that may substitute at this position include C₂-C₆ straight or branched chain alkoxy radicals, preferably ethoxy and propyloxy. C₂-C₆ straight or branched alkenoxy or C₂-C₆ alkynoxy groups may also appear at this position.

The term “aryl” denotes a single, polynuclear, conjugated or fused residue of an aromatic hydrocarbon ring system. Examples of aryl are phenyl, biphenyl and naphthyl. An aryl group may be optionally substituted by one or more substituents as herein defined. Accordingly, “aryl” as used herein also refers to a substituted aryl.

The present compounds include the following substituents for R in R¹ of Formulas I/II, when R¹ represents OR:: lower alkyl, lower alkenyl, lower alkynyl, optionally substituted lower alkyl, alkenyl, or alkynyl; lower alkoxy, alkenoxy and alkynoxy; straight and branched alkylamines, alkenyl amines and alkynyl amines (wherein the N may be tertiary or quatenary).

Most preferred R¹ groups are 3-6 member heterocyclic groups, preferably heteroaryl group with a single N heteroatom. Most preferred are 3 member (aziridinyl) and 4 member (azetidinyl) heteroaryl rings. Also preferred are larger rings, including, pyridyl, pyrrolyl, piperidinyl, etc.

More broadly, the term “heteroaryl” denotes a single, polynuclear, conjugated or fused aromatic heterocyclic ring system, wherein one or more carbon atoms of a cyclic hydrocarbon residue is substituted with a heteroatom to provide a heterocyclic aromatic residue. Where two or more carbon atoms are replaced, the replacing atoms may be two or more of the same heteroatom or two different heteroatoms. Besides N, suitable heteroatoms include O, S and Se. The heterocyclic rings may include single and double bonds. Examples of groups within the scope of this invention are those with other heteroatoms, fused rings, etc., include thienyl, furyl, indolyl, imidazolyl, oxazolyl, pyridazinyl, pyrazolyl, pyrazinyl, thiazolyl, pyrimidinyl, quinolinyl, isoquinolinyl, benzofuranyl, benzothienyl, purinyl, quinazolinyl, phenazinyl, acridinyl, benzoxazolyl, benzothiazolyl and the like. As defined herein, a heteroaryl group may be optionally further mono- or di-substituted by one or more substituents as described above at available ring positions, with, for example, lower alkyl, alkoxy, alkenyl, alkenoxy groups, etc.

In one preferred embodiment, R¹ in Formula I/II is a substituted aryl group which is substituted by one or more alkyl, carboxy, amido or amino groups, for example, —CH₃, —CH₂CH₃, —(CH₂)ₘCO₂R¹, —(CH₂)ₘCH₂OR², —(CH₂)ₘCONHR², —(CH₂)ₘNHR², —(CH₂)ₘCONR²R³ or —(CH₂)ₘCONR²R³ wherein m=0-3, R¹ is H, alkyl or aryl, and wherein R² or R³, independently, is H, alkyl, aryl or acyl. Other preferred R¹ groups in formula I include: phenyl; 2-methylphenyl; 2,4-dimethylphenyl; 2,4,6-trimethylphenyl; 2-methyl, 4-chlorophenyl; aryloxyalkyl (e.g., phenoxymethyl or phenoxyethyl); benzyl; phenethyl; 2, 3 or 4-methoxyphenyl; 2, 3 or 4-methylphenyl; 2, 3 or 4-pyridyl; 2, 4 or 5-pyrimidinyl; 2 or 3-thiophenyl; 2,4, or 5-(1,3)-oxazolyl; 2, 4 or 5-(1,3)-thiazolyl; 2 or 4-imidazolyl; 3 or 5-symtriazolyl.

An alkylene chain can be lengthened, for example, by the Arndt-Eistert synthesis wherein an acid chloride is converted to a carboxylic acid with the insertion of CH₂. Thus, a carboxylic acid group can be converted to its acid chloride derivative, for example by treatment with SO₂Cl₂. The acid chloride derivative can be reacted with diazomethane to form the diazoketone which can then be treated with Ag₂/H₂O or silver benzoate and triethylamine. The process can be repeated to further increase the length of the alkylene chain. Alternatively, an aldehyde (or keto) group could be subjected to Wittig-type reaction (using e.g., Ph₃(P)═CHCO₂Me) to produce the α,β-unsaturated ester. Hydrogenation of this double bond yields the alkylene chain that has been increased in length by two carbon atoms. In a similar manner, other phosphoranes can be used to generate longer (and optionally substituted, branched or unsaturated) carbon chains.

The present compounds include those with R² substituents of Formulas I/II that are the same as those described for R¹. Both ansamycin ring positions C17 and C19 may be independently substituted, though it is preferred that if C17 is substituted R² is H.

The R³ substituent bonded to the N at ring position 22 of Formula I/II is preferably H, (as in GA and the compounds exemplified herein), or lower alkyl, lower alkenyl, lower alkynyl, optionally substituted lower alkyl, alkenyl, or alkynyl; lower alkoxy, alkenoxy and alkynoxy; straight and branched alkylamines, alkenyl amines and alkynyl amines (wherein the N may be tertiary or quatenary). The N may be part of a heterocycloalkyl, heterocylokenyl or heteroaryl ring that is optionally substituted. If the N is part of a ring, it is preferably a 3-6 member ring, preferably with no additional heteroatoms. Most preferred are aziridinyl, azetidinyl, pyridyl, pyrrolyl, piperidinyl, etc.

Bonded to ring position C11 of Formula I/II is an O atom that is substituted with an R⁴ group. R⁴ is most preferably lower alkyl but may also be lower alkenyl, lower alkynyl, optionally substituted lower alkyl, alkenyl, or alkynyl, such that the moiety bonded to C11 is preferably an alkoxy moiety, but may also be an alkenoxy and alkynoxy moiety.

In addition to the various substituents of Formulas I/II disclosed above, the ring double bonds between positions C₂═C₃, C₄═C₅, and C₈═C₉ may be hydrogenated to single bonds.

It should be evident that chemical manipulation of a substituent at certain positions in the ring Formula I/II may require protection of other potentially reactive groups. Suitable protective groups for use under the appropriate conditions, as well as methods for their introduction and removal are well-known in the art and are described in Greene T W et al., Protective Groups in Organic Synthesis, 3^{rd} ed, John Wiley and Son, 1999, the contents of which are incorporated herein by reference.

The compound of the present invention may optionally be bound to, or include in its substituted ring structure, a radionuclide that is diagnostically or therapeutically useful. (See below). The compound may be bound to a targeting moiety that binds specifically to a protein.

In one embodiment of the present invention, in view of WO98/51702 (supra), the GA derivative of the present invention (whether free or detectably labeled to bound to a targeting moiety) is a compound as described herein, with the proviso that the compound is not GA (compound), compound 15; or 17-(N-iodoethyl-N-cyano-17-demethoxygeldanamycin (with or without a radioactive iodine). However, embodiments of the present methods may encompass such excluded compounds based on the fact that the uses of the present invention were not disclosed in that reference.

In another embodiment of the present invention, in view of WO95/01342 (supra), the GA derivative whether free or bound to a targeting moiety or labeled with a detectable label to compound of the present invention is a compound as described herein with the proviso that the compound is not one disclosed in WO95/01342, specifically, the compounds listed beginning at page 15, line 19, through page 17, line 12, or Examples 2-99. Example 21 of this reference discloses present compound 8, but, does not suggest its novel property of being active against tumor cells at a fM or sub-fm concentrations.

In another embodiment of the present invention, in view of U.S. Pat. No. 5,932,566 (Schnur et al., supra) the GA derivative whether free, detectably labeled, or bound to a targeting moiety, is a compound as described herein with the proviso that the compound is not:
17-amino-4,5-dihydro-17-demethoxygeldanamycin;
17-methylamino-4,5-dihydro-17-demethoxygeldanamycin;
17-cyclopropylamino-4,5-dihydro-17-demethoxygeldanamycin;
17-(2′-Hydroxyethylamino)-4,5-dihydro-17-demethoxygelclanamycin;
17-(2-Methoxyethylamino)-4,5-dihydro-17-demethoxygeldanamycin;
17-(2′-Fluoroethylamino)-4,5-dihydro-17-demethoxygeldanamycin;
17-s-(+)-2-Hydroxypropylamino!-4,5-dihydro-17-demethoxygeldanamycin;
17-azetidin-1-yl-4,5-dihydro-17-demethoxygeldanamycin;
17-(3-hydroxyazetidin-1-yl)-4,5-dihydro-17-demethoxygeldanamycin;
17-azetidin-1-yl-4,5-dihydro-11-α-fluoro-17-demethoxygeldanamycin;
17-azetidin-1-yl-17-demethoxygeldanamycin;
17-(2′-cyanoethylamino)-17-demethoxygeldanamycin;
17-(2′-fluoroethylamino)-17-demethoxygeldanamycin;
17-amino-22-(2′-methoxyphenacyl)-17-demethoxygeldanamycin;
17-amino-22-(3′-methoxyphenacyl)-17-demethoxygeldanetmycin;
17-amino-22-(4′-chlorophenacyl)-17-demethoxygeldanamycin;
17-amino-22-(3′,4′-dichlorophenacyl)-17-demethoxygeldanamycin;
17-amino-22-(4′-amino-3′-iodophenacyl)-17-demethoxygeldanamycin;
17-amino-22-(4′-azido-3′-iodophenacyl)-17-demethoxygeldanamycin;
17-amino-11-α-fluoro-17-demethoxygeldanamycin;
17-allylamino-11-α-fluoro-17-demethoxygeldanamycin;
17-propargylamino-11-α-fluoro-17-demethoxygeldanamycin;
17-(2′-fluoroethylamino)-11-α-fluoro-17-demethoxygeldanamycin;
17-azetidin-1-yl-11-(4′-azidophenyl)sulfamylcarbonyl-17-demethoxygeldanamycin;
17-(2′-Fluoroethylamino)-11-keto-17-demethoxygeldanamycin;
17-azetidin-1-yl-11-keto-17-demethoxygeldanamycin; and
17-(3′-hydroxyazetidin-1-yl)-11-keto-17-demethoxygeldanamycin.

In another embodiment of the present invention, in view of WO 2004/087045 (supra) the GA derivative whether free or bound to a targeting moiety or labeled with a detectable label is a compound as described herein with the proviso that the compound is not 17-allylamino-17-demethoxygeldanamycin; 17-2-dimethylamino)ethylamino]-demethoxy-11-O-methylgeldanamycin; or 17-N-Azetidinyl-17. However, embodiments of the present methods may encompass such excluded compounds based on the fact that the uses of the present invention were not disclosed in that reference.

Radiolabeled GA Derivatives for Imaging

A preferred composition is a detectably or diagnostically labeled GA derivative compound of the present invention to which is covalently bound a detectable label that is preferably one that is imageable in vivo. Preferred detectable labels are radionuclides, in particular, halogen atoms that can be readily attached to the GA derivative.

The chemistry of substituting a halogen group X(═F, Br, Cl, I) by using the HX acid to open a N-containing heterocyclic ring such as the aziridine ring of a GA derivative, in particular 17-(1-aziridinyl)-17-demethoxygeldanamycin (“17-ARG”) which is compound 15 herein, is relatively straightforward (See Example 19) for details of making fluoro, chloro, bromo and iodo forms of this GA derivative. The radionuclide atom is covalently bonded. Such halogenated GA derivatives can be useful imaging agents in vivo, for experimental animal models and humans, for research, diagnosis and prognosis.

In a preferred embodiment, a 17-(2-haloethyl)amino-17-demethoxygeldanamycin is are made as described in Example 19, by reacting 15 with radioactive HX* acid ((wherein X*=¹⁸F, ⁷⁶Br, ⁷⁶Br, ¹²³I, ¹²⁴I, ¹²⁵I, or ¹³¹I).

A summary of the properties of some of these nuclides appears below (some taken from Vallabhajosula, S, Radiopharmaceuticals in Oncology, Chapter 3, Nuclear Oncology: Diagnosis and Thzerapy (I Khalkhali et al., eds) Lippincott, Williams & Wilkins, Philadelphia, 2001, p. 33)

HALOGEN RADIONUCLIDES FOR DIAGNOSTIC USES

| | Half-life | | Photon energy | Abundance τ |
|---|---|---|---|---|
| Nuclide | (h) | Decay mode | (keV) | emission (%) |
| ¹³¹I | 193 | β−, τ | 364 | 81 |
| ¹²³I | 13 | EC | 159 | 83 |
| | | | 33 (Te x-rays) | |

| | Half-life | Decay | Energy (MeV) | Max Range | τ Photon | |
|---|---|---|---|---|---|---|
| Nuclide | (d) | mode | Max/Avg | in Tissue | (Mev) | |
| ¹³¹I | 8.04 | β⁻, τ | 0.61/0.20 | 2.4 mm | 364 | Mev |
| ¹²⁵I | 60.3 | EC | 0.4 keV (Auger e⁻) | 10.0 μm | 25-35 | keV |

POSITRON-EMITTING RADIONUCLIDES (for PET Imaging)

| | |
|---|---|
| Energy of Particles | β⁺ |

| | Half | | | Max β+ | | Range |
|---|---|---|---|---|---|---|
| Nuclide | Life | | Decay modes | Energy | Photon | (mm) |
| ¹⁸F | 110 | min | 96.9 β+ | 0.63 | 0.511 | 2.4 |
| ⁷⁶Br | 16.2 | hr | 57% β+ | 3.98 MeV | | |
| | | | (18 mm positron | | | |
| | | | range) 43% EC | | | |
| | | | 0.68 Auger | | | |
| | | | e⁻/decay | | | |
| ⁷⁷Br | 2.4 | d | 0.74% β+ | 0.36 MeV | | |
| | | | (0.2 mm positron | | | |
| | | | range) 99.3% EC | | | |
| | | | 0.85 Conversion | | | |
| | | | e⁻/decay | | | |
| ¹²⁴I | 4.2 | d | 25% β+ | 2.14 MeV | 0.511 | |
| | | | (10 mm positron | | | |
| | | | range) 75% EC | | | |
| | | | 0.713 Auger | | | |
| | | | e−/decay | | | |

EC, Electron capture
¹²⁵I and ¹³¹I are two additional radionuclides; both have potential therapeutic as well as diagnostic utility.
¹²⁵I decays by electron capture and emits Auger electrons as well as β irradiation.
¹³¹I is a β emitter.
¹²⁵I is particularly useful in small animal imaging, for example, to image tumors, by scintigraphy or Single photon emission computed tomography (SPECT). For a general description of SPECT, see: Heller, S. L. et al., Sem. Nucl. Med. 17: 183-199 (1987); Cerquiera, M. D. et al., Sem. Nucl. Med. 17: 200-213 (1987); Ell, P. J. et al., Sem. Nucl. Med. 17: 214-219 (1987)).
¹²³I, radionuclide used for in vivo imaging does not emit particles, but produces a large number of photons in a 140-200 keV range, which may be readily detected by conventional gamma cameras.

These types of labels permits detection or quantitation of the Met bearing cells in a tissue sample and can be used, therefore, as a diagnostic and a prognostic tool in a disease where expression or enhanced expression of Met (or its binding of HGF) plays a pathological or serves as a diagnostic marker and/or therapeutic target, particularly, cancer.

Preferred diagnostic methods are thus PET imaging, scintigraphic analysis, and SPECT. These can performed in a manner that results in serial total body images and allows determination of regional activity by quantitative “region-of-interest” (ROI) analysis.

Examples of imaging procedures and analysis, especially for animal models, are described in Gross M D et al. (1984) Invest Radiol 19:530-534; Hay R V et al. (1997) Nucl Med Commun 18:367-378).

Pharmaceutical Compositions, Their Formulation and Use

The compounds of Formula I/II and their pharmaceutically acceptable salts are useful as unusually highly potent antitumor/anticancer agents and appear to act by inhibiting certain cellular interactions between, or subsequent to binding of, HGF/SF and its receptor, Met. They may also be useful in inhibiting other growth factor/receptor interactions s that play an important role in uncontrolled cell proliferation, such as the EGF receptor, the NGF receptor, the PDGF receptor and the insulin receptor.

A pharmaceutical composition according to this invention comprises the FM-GAi compound in a formulation that, as such, is known in the art. Pharmaceutical compositions within the scope of this invention include all compositions wherein the fM-GAi compound is contained in an amount effective to achieve its intended purpose. While individual needs vary, determination of optimal ranges of effective amounts of each component is within the skill of the art. Typical dosages comprise 0.01 pg to 100 μg/kg/body mass, more preferably 1 pg to 100 μg/kg body mass, more preferably 10 pg-10 μg/kg body mass.

In addition to the pharmacologically active molecule, the pharmaceutical compositions may contain suitable pharmaceutically acceptable carriers comprising excipients and auxiliaries which facilitate processing of the active compounds into preparations which can be used pharmaceutically as is well known in the art. Suitable solutions for administration by injection or orally, may contain from about 0.01 to 99 percent, active compound(s) together with the excipient.

The pharmaceutical preparations of the present invention are manufactured in a manner which is known, for example, by means of conventional mixing, granulating, dissolving, or lyophilizing processes. Suitable excipients may include fillers binders, disintegrating agents, auxiliaries and stabilizers, all of which are known in the art. Suitable formulations for parenteral administration include aqueous solutions of the proteins in water-soluble form, for example, water-soluble salts. Compounds are preferably be dissolved in dimethylsulfoxide (DMSO) and administered intravenously (i.v.) as a DMSO solution mixed into an aqueous i.v. formulation (see Goetz J P et al., 2005, J. Clin. Oncol. 2005, 23:1078-1087, for a description of the administration of 17-allylamino-17-demethoxygeldanamycin. Another compound, 17-(2-dimethylaminoethyl)amino-17-demethoxygeldanamycin can be given i.v. in DMSO as above, or orally in a different formulation. For the compounds and methods of the present invention, a preferred solvent is DMSO further diluted into a standard aqueous i.v. solution.

In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension.

The compositions may be in the form of a lyophilized particulate material, a sterile or aseptically produced solution, a tablet, an ampule, etc. Vehicles, such as water (preferably buffered to a physiologically acceptable pH, as for example, in phosphate buffered saline) or an appropriate organic solvent, other inert solid or liquid material such as normal saline or various buffers may be present. The particular vehicle is not critical, and those skilled in the art will know which vehicle to use for any particular utility described herein.

In general terms, a pharmaceutical composition is prepared by mixing, dissolving, binding or otherwise combining the polymer or polymeric conjugate of this invention with one or more water-insoluble or water-soluble aqueous or non-aqueous vehicles. It is imperative that the vehicle, carrier or excipient, as well as the conditions for formulating the composition are such that do not adversely affect the biological or pharmaceutical activity of the active compound.

Subjects, Treatments Modes and Routes of Administration

The preferred animal subject of the present invention is a mammal. The invention is particularly useful in the treatment of human subjects. By the term “treating” is intended the administering to subjects of a pharmaceutical composition comprising a fM-GAi compound. Treating includes administering the agent to subjects at risk for developing a Met-positive tumor prior to evidence of clinical disease, as well as subjects diagnosed with such tumors or cancer, who have not yet been treated or who have been treated by other means, e.g., surgery, conventional chemotherapy, and in whom tumor burden has been reduced even to the level of not being detectable. Thus, this invention is useful in preventing or inhibiting tumor primary growth, recurrent tumor growth, invasion and/or metastasis or metastatic growth.

The pharmaceutical compositions of the present invention wherein the fM-GAi compound is combined with pharmaceutically acceptable excipient or carrier, may be administered by any means that achieve their intended purpose. Amounts and regimens for the administration of can be determined readily by those with ordinary skill in the clinical art of treating any of the particular diseases. Preferred amounts are described below.

The active compounds of the invention may be administered orally, topically, parenterally, by inhalation spray or rectally in dosage unit formulations containing conventional non-toxic pharmaceutically acceptable carriers, adjuvants and vehicles.

In general, the present methods include administration by parenteral routes, including injection or infusion using any known and appropriate route for the subject's disease and condition. Parenteral routes include subcutaneous (s.c.) intravenous (i.v.), intramuscular, intraperitoneal, intrathecal, intracisternal transdermal, topical, rectal or inhalational. Also included is direct intratumoral injection. Alternatively, or concurrently, administration may be by the oral route. The dosage administered will be dependent upon the age, health, and weight of the recipient, kind of concurrent treatment, if any, frequency of treatment, and the nature of the effect desired. Preferably the active compound of the invention is administered in a dosage unit formulation containing conventional non-toxic pharmaceutically acceptable carriers, adjuvants and vehicles.

In one treatment approach, the compounds and methods are applied in conjunction with surgery. Thus, an effective amount of the fM-GAi compound is applied directly to the site of surgical removal of a tumor (whether primary or metastatic). This can be done by injection or “topical” application in an open surgical site or by injection after closure.

In one embodiment, a specified amount of the compound, preferably about 1 pg-100 μg, is added to about 700 ml of human plasma that is diluted 1:1 with heparinized saline solution at room temperature. Human IgG in a concentration of 500 μg/dl (in the 700 ml total volume) may also be used. The solutions are allowed to stand for about 1 hour at room temperature. The solution container may then be attached directly to an iv infusion line and administered to the subject at a preferred rate of about 20 ml/min.

In another embodiment, the pharmaceutical composition is directly infused i.v. into a subject. The appropriate amount, preferably about 1 pg-100 μg, is added to about 250 ml of heparinized saline solution and infused iv into patients at a rate of about 20 ml/min.

The composition can be given one time but generally is administered six to twelve times (or even more, as is within the skill of the art to determine empirically). The treatments can be performed daily but are generally carried out every two to three days or as infrequently as once a week, depending on the beneficial and any toxic effects observed in the subject. If by the oral route, the pharmaceutical composition, preferably in a convenient tablet or capsule form, may be administered once or more daily.

The pharmaceutical formulation for systemic administration according to the invention may be formulated for enteral, parenteral or topical administration, and all three types of formulation may be used simultaneously to achieve systemic administration of the active ingredient.

For lung instillation, aerosolized solutions are used. In a sprayable aerosol preparations, the active protein or small molecule agent may be in combination with a solid or liquid inert carrier material. This may also be packaged in a squeeze bottle or in admixture with a pressurized volatile, normally gaseous propellant. The aerosol preparations can contain solvents, buffers, surfactants, and antioxidants in addition to the protein of the invention.

The appearance of tumors in sheaths (“theca”) encasing an organ often results in production and accumulation of large volumes of fluid in the organ's sheath. Examples include (1) pleural effusion due to fluid in the pleural sheath surrounding the lung, (2) ascites originating from fluid accumulating in the peritoneal membrane and (3) cerebral edema due to metastatic carcinomatosis of the meninges. Such effusions and fluid accumulations generally develop at an advanced stage of the disease. The present invention contemplates administration of the pharmaceutical composition directly administration into cavities or spaces, e.g., peritoneum, thecal space, pericardial and pleural space containing tumor. That is the agent is directly administered into a fluid space containing tumor cells or adjacent to membranes such as pleural, peritoneal, pericardial and thecal spaces containing tumor. These sites display malignant ascites, pleural and pericardial effusions or meningeal carcinomatosis. The drug is preferably administered after partial or complete drainage of the fluid (e.g., ascites, pleural or pericardial effusion) but it may also be administered directly into the undrained space containing the effusion, ascites and/or carcinomatosus. In general, the fM-CAi compound's dose may vary from 1 femtogram to 10 μg, preferably, 1 pg to 1 μg, and given every 3 to 10 days. It is continued until there is no reaccumulation of the ascites or effusion. Therapeutic responses are considered to be no further accumulation of four weeks after the last intrapleural administration.

For topical application, the active compound may be incorporated into topically applied vehicles such as salves or ointments, as a means for administering the active ingredient directly to the affected area. Scarification methods, known from studies of vaccination, can also be used. The carrier for the active agent may be either in sprayable or nonsprayable form. Non-sprayable forms can be semi-solid or solid forms comprising a carrier indigenous to topical application and having a dynamic viscosity preferably greater than that of water. Suitable formulations include, but are not limited to, solution, suspensions, emulsions, creams, ointments, powders, liniments, salves, and the like. If desired, these may be sterilized or mixed with auxiliary agents, e.g., preservatives, stabilizers, wetting agents, buffers, or salts for influencing osmotic pressure and the like. Examples of preferred vehicles for non-sprayable topical preparations include ointment bases, e.g., polyethylene glycol-1000 (PEG-1000); conventional creams such as HEB cream; gels; as well as petroleum jelly and the like.

Other pharmaceutically acceptable carriers according to the present invention are liposomes or other timed-release or gradual release carrier or drug delivery device known in the art

Combinations with Chemotherapeutic and Biological Anti-Cancer Agents

Chemotherapeutic agents can be used together with the present compounds, by any conventional route and at doses readily determined by those of skill in the art. Anti-cancer chemotherapeutic drugs useful in this invention include but are not limited to antimetabolites, anthracycline, vinca alkaloid, anti-tubulin drugs, antibiotics and alkylating agents. Representative specific drugs that can be used alone or in combination include cisplatin (CDDP), adriamycin, dactinomycin, mitomycin, caminomycin, daunomycin, doxorubicin, tamoxifen, taxol, taxotere, vincristine, vinblastine, vinorelbine, etoposide (VP-16), verapamil, podophyllotoxin, 5-fluorouracil (5FU), cytosine arabinoside, cyclophosphamide, thiotepa, methotrexate, camptothecin, actinomycin-D, mitomycin C, aminopterin, combretastatin(s) and derivatives and prodrugs thereof.

Any one or more of such drugs, newer drugs targeting oncogene signal transduction pathways, or that induce apoptosis or inhibit angiogenesis, and biological products such as nucleic acid molecules, vectors, antisense constructs, siRNA constructs, and ribozymes, as appropriate, may be used in conjunction with the present compounds and methods. Examples of such agents and therapies include, radiotherapeutic agents, antitumor antibodies with attached anti-tumor drugs such as plant-, fungus-, or bacteria-derived toxin or coagulant, ricin A chain, deglycosylated ricin A chain, ribosome inactivating proteins, sarcins, gelonin, aspergillin, restricticin, a ribonuclease, a epipodophyllotoxin, diphtheria toxin, or Pseudomonas exotoxin. Additional cytotoxic, cytostatic or anti-cellular agents capable of killing or suppressing the growth or division of tumor cells include anti-angiogenic agents, apoptosis-inducing agents, coagulants, prodrugs or tumor targeted forms, tyrosine kinase inhibitors, antisense strategies, RNA aptamers, siRNA and ribozymes against VEGF or VEGF receptors. Any of a number of tyrosine kinase inhibitors are useful when administered together with, or after, the present compounds. These include, for example, the 4-aminopyrrolo[2,3-d]pyrimidines (U.S. Pat. No. 5,639,757). Further examples of small organic molecules capable of modulating tyrosine kinase signal transduction via the VEGF-R2 receptor are the quinazoline compounds and compositions (U.S. Pat. No. 5,792,771). Other agents which may be employed in combination with the present invention are steroids such as the angiostatic 4,9(11)-steroids and C²¹-oxygenated steroids (U.S. Pat. No. 5,972,922).

Thalidomide and related compounds, precursors, analogs, metabolites and hydrolysis products (U.S. Pat. Nos. 5,712,291 and 5,593,990) may also be used in combination to inhibit angiogenesis. These thalidomide and related compounds can be administered orally. Other anti-angiogenic agents that cause tumor regression include the bacterial polysaccharide CM101 (currently in clinical trials) and the antibody LM609. CM101 induces neovascular inflammation in tumors and downregulates expression VEGF and its receptors. Thrombospondin (TSP-1) and platelet factor 4 (PF4) are angiogenesis inhibitors that associate with heparin and are found in platelet α granules. Interferons and matrix metalloproteinase inhibitors (MMPI's) are two other classes of naturally occurring angiogenic inhibitors that can be used. Tissue inhibitors of metalloproteinases (TIMPs) are a family of naturally occurring MMPI's that also inhibit angiogenesis. Other well-studied anti-angiogenic agents are angiostatin, endostatin, vasculostatin, canstatin and maspin.

Chemotherapeutic agents are administered as single agents or multidrug combinations, in full or reduced dosage per treatment cycle. The combined use of the present compositions with low dose, single agent chemotherapeutic drugs is particularly preferred. The choice of chemotherapeutic drug in such combinations is determined by the nature of the underlying malignancy. For lung tumors, cisplatin is preferred. For breast cancer, a microtubule inhibitor such as taxotere is the preferred. For malignant ascites due to gastrointestinal tumors, 5-FU is preferred. “Low dose” as used with a chemotherapeutic drug refers to the dose of single agents that is 10-95% below that of the approved dosage for that agent (by the U.S. Food and Drug Administration, FDA). If the regimen consists of combination chemotherapy, then each drug dose is reduced by the same percentage. A reduction of >50% of the FDA approved dosage is preferred although therapeutic effects are seen with dosages above or below this level, with minimal side effects. Multiple tumors at different sites may be treated by systemic or by intrathecal or intratumoral administration of the fM-GAi compound.

The optimal chemotherapeutic agents and combined regimens for all the major human tumors are set forth in Bethesda Handbook of Clinical Oncology, Abraham J et al., Lippincott William & Wilkins, Philadelphia, Pa. (2001); Manual of Clinical Oncology, Fourth Edition, Casciato, D A et al, Lippincoft William & Wilkins, Philadelphia, Pa. (2000) both of which are herein incorporated in entirety by reference.

In Vivo Testing of fM-GAi Compounds

The fM-GAi compound may be tested for therapeutic efficacy in well established rodent models which are considered to be representative of a human tumor. The overall approach is described in detail in Geran, R. I. et al, “Protocols for Screening Chemical Agents and Natural Products Against Animal Tumors and Other Biological Systems (3d Ed)”, Canc. Chemother. Reports, Part 3, 3:1-112; and Plowman, J et al., In: Teicher, B, ed., Anticancer Drug Development Guide: Preclinical Screening, Clinical Trials and Approval, Part II: In Vivo Methods, Chapter 6, “Human Tumor Xenograft Models in NCI Drug Development,” Humana Press Inc., Totowa, N.J., 1997. Both these references are hereby incorporated by reference in their entirety.

Human Tumor Xenograft Models

The preclinical discovery and development of anticancer drugs as implemented by the National Cancer Institute (NCI) consists of a series of test procedures, data review, and decision steps (Grever, M R, Semin Oncol., 19:622-638 (1992)). Test procedures are designed to provide comparative quantitative data, which in turn, permit selection of the best candidate agents from a given chemical or biological class. Below, we describe human tumor xenograft systems, emphasizing melanomas, that are currently employed in preclinical drug development.

Since 1975, the NCI approach to drug discovery involved prescreening of compounds in the i.p.-implanted murine P388 leukemia model (see above), followed by evaluation of selected compounds in a panel of transplantable tumors (Venditti, J. M. et al., In: Garrattini S et al, eds., Adv. Pharmacol and Chemother 2:1-20 (1984)) including human solid tumors. The latter was made possible through the development of immunodeficient athymic nude (nu/nu) mice and the transplantation into these mice of human tumor xenografts (Rygaard, J. et al, Acta Pathol. Microbiol. Scand. 77:758-760 (1969); Giovanella, G. C. et al, J. Natl Canc. Inst. 51:615-619 (1973)). Studies assessing the metastatic potential of selected murine and human tumor-cell lines (B16, A-375, LOX-IMVI melanomas, and PC-3 prostate adenocarcinoma) and their suitability for experimental drug evaluation supported the importance of in vivo models derived from the implantation of tumor material in anatomically appropriate host tissues; such models are well suited for detailed evaluation of compounds that inhibit activity against specific tumor types. Beginning about 1990, the NCI began employing human tumor cell lines for large-scale drug screening ((Boyd, M R, In: DeVita, V T et al., Cancer: Principles and Practice of Oncology, Updates, vol 3, Philadelphia, Lippinicott, 1989, pp 1-12; Plowman, supra). Cell lines derived from seven cancer types (brain, colon, leukemia, lung, melanoma, ovarian, and renal) were acquired from a wide range of sources, frozen, and subjected to a battery of in vitro and in vivo characterization. This approach shifted the screening strategy from “compound-oriented” to “disease-oriented” drug discovery (Boyd, supra). Compounds of identified by the screen, demonstrating disease-specific, differential cytotoxicity were considered “leads” for further preclinical evaluation. A battery of human tumor xenograft models was created to deal with such needs.

The initial solid tumors established in mice are maintained by serial passage of 30-40 mg tumor fragments implanted s.c. near the axilla. Xenografts are generally not utilized for drug evaluation until the volume-doubling time has stabilized, usually around the fourth or fifth passage.

The in vivo growth characteristics of the xenografts determine their suitability for use in the evaluation of test agent antitumor activity, particularly when the xenografts are utilized as early stage s.c. models. As used herein, an early stage s.c. model is defined as one in which tumors are staged to 63-200 mg prior to the initiation of treatment. Growth characteristics considered in rating tumors include take-rate, time to reach 200 mg, doubling time, and susceptibility to spontaneous regression. As can be noted, the faster-growing tumors tend to receive the higher ratings.

Any of a number of transgenic mouse models known in the art can be used to test the present compounds. A particularly useful murine human HGF/SF transgenic model has been described by one of the present inventors and his colleagues and may be used to test the present compounds against human tumor xenografts in vivo. See, Zhang Y W et al. (2005) Oncogene 24:101-106; U.S. Pat. App Ser. No. 60/587,044, which references are incorporated by reference in their entirety. Other longer-known models are described below.

Advanced-Stage Subcutaneous Xenograft Models

Such s.c.-implanted tumor xenograft models are used to evaluate the antitumor activity of test agents under conditions that permit determination of clinically relevant parameters of activity, such as partial and complete regression and duration of remission (Martin D S et al., Cancer Treat Rep 68:37-38 (1984); Martin D S et al., Cancer Res. 46:2189-2192 (1986); Stolfi, R L et al., J. Natl Canc Inst 80:52-55 (1988)). Tumor growth is monitored and test agent treatment is initiated when tumors reach a weight range of 100-400 mg (staging day, median weights approx. 200 mg), although depending on the xenograft, tumors may be staged at larger sizes. Tumor sizes and body weights are obtained approximately 2 times/wk. Through software programs (developed by staff of the Information Technology Branch of DTP of the NCI), data are stored, various parameters of effects are calculated, and data are presented in both graphic and tabular formats. Parameters of toxicity and antitumor activity are defined as follows:
1. Toxicity: Both drug-related deaths (DRD) and maximum percent relative mean net body weight losses are determined. A treated animal's death is presumed to be treatment-related if the animal dies within 15 d of the last treatment, and either its tumor weight is less than the lethal burden in control mice, or its net body weight loss at death is 20% greater than the mean net weight change of the controls at death or sacrifice. A DRD also may be designated by the investigator. The mean net body weight of each group of mice on each observation day is compared to the mean net body weight on staging day. Any weight loss that occurs is calculated as a percent of the staging day weight. These calculations also are made for the control mice, since tumor growth of some xenografts has an adverse effect on body weight.
2. Optimal % T/C: Changes in tumor weight (A weights) for each treated (T) and control (C) group are calculated for each day tumors are measured by subtracting the median tumor weight on the day of first treatment (staging day) from the median tumor weight on the specified observation day. These values are used to calculate a percent T/C as follows:

$$\begin{matrix}\% T / C = ( Δ T / Δ C ) \times 100 where Δ T > 0 or \\ = ( Δ T / {T}_{I} ) \times 100 where Δ T < 0\end{matrix} \qquad ( 1 )$$
and T₁ is the median tumor weight at the start of treatment. The optimum (minimum) value obtained after the end of the first course of treatment is used to quantitate antitumor activity.
3. Tumor growth delay: This is expressed as a percentage by which the treated group weight is delayed in attaining a specified number of doublings; (from its staging day weight) compared to controls using the formula:
   [(T−C)/C]×100 (2)
where T and C are the median times (in days) for treated and control groups, respectively, to attain the specified size (excluding tumor-free mice and DRDs). The growth delay is expressed as percentage of control to take into account the growth rate of the tumor since a growth delay based on (T−C) alone varies in significance with differences in tumor growth rates.
4. Net log cell kill: An estimate of the number of log₁₀ units of cells killed at the end of treatment is calculated as:
   {[(T−C)−duration of treatment]×0.301/median doubling time} (3)
where the “doubling time” is the time required for tumors to increase in size from 200 to 400 mg, 0.301 is the log₁₀ of 2, and T and C are the median times (in days) for treated and control tumors to achieve the specified number of doublings. If the duration of treatment is 0, then it can be seen from the formulae for net log cell kill and percent growth delay that log cell kill is proportional to percent growth delay. A log cell kill of 0 indicates that the cell population at the end of treatment is the same as it was at the start of treatment. A log cell kill of +6 indicates a 99.9999% reduction in the cell population.
5. Tumor regression: The importance of tumor regression in animal models as an end point of clinical relevance has been propounded by several investigators (Martin et al., 1984, 1986 supra; Stolfi et al., supra). Regressions are defined-as partial if the tumor weight decreases to 50% or less of the, tumor weight at the start of treatment without dropping below 63 mg (5×5 mm tumor). Both complete regressions (CRs) and tumor free survivors are defined by instances in which the tumor burden falls below measurable limits (<63 mg) during the experimental period. The two parameters differ by the observation of either tumor regrowth (in CR animals) or no regrowth (=tumor-free) prior to the final observation day. Although one can measure smaller tumors, the accuracy of measuring a s.c. tumor smaller than 4×4 mm or 5×5 mm (32 and 63 mg, respectively) is questionable. Also, once a relatively large tumor has regressed to 63 mg, the composition of the remaining mass may be only fibrous material/scar tissue. Measurement of tumor regrowth following cessation of treatment provides a more reliable indication of whether or not tumor cells survived treatment.

Most xenografts that grow s.c. may be used in an advanced-stage model, although for some tumors, the duration of the study may be limited by tumor necrosis. As mentioned previously, this model enables the measurement of clinically relevant parameters and provides a wealth of data on the effects of the test agent on tumor growth. Also, by staging day, the investigator is ensured that angiogenesis has occurred in the area of the tumor, and staging enables “no-takes” to be eliminated from the experiment. However, the model can be costly in terms of time and mice. For slower-growing tumors, the passage time required before sufficient mice can be implanted with tumors may be at least ˜4 wks, and an additional 2-3 wks may be required before the tumors can be staged. To stage tumors, more mice (as many as 50-100% more) than are needed for actual drug testing must be implanted.

Early Treatment and Early Stage Subcutaneous Xenograft Models

These models are similar to the advanced-stage model, but, because treatment is initiated earlier in the development of the tumor, useful tumors are those with >90% take-rate (or <10% spontaneous regression rate). The “early treatment model” is defined as one in which treatment is initiated before tumors are measurable, i.e., <63 mg. The “early stage” model as one in which treatment is initiated when tumor size ranges from 63-200 mg. The 63-mg size is used because it indicates that the original implant, about 30 mg, has demonstrated some growth. Parameters of toxicity are the same as those for the advanced-stage model; parameters of antitumor activity are similar. % T/C values are calculated directly from the median tumor weights on each observation day instead of being measured as changes (Δ) in tumor weights, and growth delays are based on the days after implant required for the tumors to reach a specified size, e.g., 500 or 1000 mg. Tumor-free mice are recorded, but may be designated as “no-takes” or spontaneous regressions if the vehicle-treated control group contains >10% mice with similar growth characteristics. A “no-take” is a tumor that fails to become established and grow progressively. A spontaneous regression (graft failure) is a tumor that, after a period of growth, decreases to ≦50% of its maximum size. Tumor regressions are not normally recorded, since they are not always a good indicator of antineoplastic effects in the early stage model. A major advantage of the early treatment model is the ability to use all implanted mice, which is why a good tumor take-rate is required. In practice, the tumors most suitable for this model tend to be the faster-growing ones.

Challenge Survival Models

In another approach, the effect of human tumor growth on the lifespan of the host is determined. All mice dying or sacrificed owing to a moribund state or extensive ascites prior to the final observation day are used to calculate median day of death for treated (T) and control (C) groups. These values are then used to calculate a percent increase in life span (“ILS”) as follows:
% ILS=[(T−C/C]×100 (4)

Where possible, titration groups are included to establish a tumor doubling time for use in log₁₀ cell kill calculations. A death (or sacrifice) may be designated as drug-related based on visual observations and/or the results of necropsy. Otherwise, treated animal deaths are-designated as treatment-related if the day of death precedes the mean day of death of the controls (−2SD) or if the animal dies without evidence of tumor within 15 days of the last treatment.

Response of Xenograft Models to Standard Agents

In obtaining drug sensitivity profiles for the advanced-stage s.c. xenograft models, the test agent is evaluated following i.p. administration at multiple dose levels. The activity ratings are based on the optimal effects attained with the maximally tolerated dose (<LD₂₀) of each drug for a given treatment schedule which is selected on the basis of the doubling time of a given tumor, with longer intervals between treatments for slower growing tumors.

As described in Plowman, J. et al., supra, at least minimal antitumor effects (% T/C≦40) were produced in the melanoma group by at least 2, and as many as 10, clinical drugs. The number of responses appeared to be independent of doubling time and histological type with a range in the number of responses observed for tumors (seen in each subpanel of other tumor types as well). When the responses are considered in terms of the more clinically relevant end points of partial or complete tumor regression, these tumors models (across all tumors) were quite refractory to standard drug therapy; the tumors did not respond to any of the drugs tested in 30 of 48 (62.5%) of all tumors.

Strategy for Initial Compound Evaluation in Vivo

The in vitro primary screens provide a basis for selecting the most appropriate tumor lines to use for follow-up in vivo testing, with each compound tested only against xenografts derived from cell lines demonstrating the greatest sensitivity to the agent in vitro. The early strategy for in vivo testing emphasized the treatment of animals bearing advanced-stage tumors.

Based on the specific information available to guide dose selection here, much lower doses than those used for typical test agents are selected. Single mice are preferably treated with single ip bolus doses of between 1 pg/kg and 1 mg/kg and observed for 14 d. Sequential 3-dose studies may be conducted as necessary until a nonlethal dose range is established. The test agent is then evaluated preferably in three s.c. xenograft models using tumors that are among the most sensitive to the test agent in vitro and that are suitable for use as early stage models. The compounds are administered ip, as suspensions if necessary, on schedules based, with some exceptions, on the mass doubling time of the tumor. For example, for doubling times of 1.3-2.5, 2.6-5.9, and 6-10 d, preferred schedules are: daily for five treatments (qd×5), every fourth day for three treatments (q4d×3), and every seventh day for three treatments (q7d×3). For most tumors, the interval between individual treatments approximates the doubling time of the tumors, and the treatment period allows a 0.5-1.0 log₁₀ unit of control tumor growth. For tumors staged at 100-200 mg, the tumor sizes of the controls at the end of treatment should range from 500-2000 mg, which allows sufficient time after treatment to evaluate the effects of the test agent before it becomes necessary to sacrifice mice owing to tumor size.

Detailed Drug Studies

Once a compound has been identified as demonstrating in vivo efficacy in initial evaluations, more detailed studies are designed and conducted in human tumor xenograft models to explore further the compound's therapeutic potential. By varying the concentration and exposure time of the tumor cells and the host to the drug, it is possible to devise and recommend treatment strategies designed to optimize antitumor activity.

The importance of “concentration×time” on the antitumor effects of test agents were well illustrated by data obtained with amino-20M-camptothecin (Plowman, J. et al., 1997, supra). Those results indicated that maintaining the plasma concentration above a threshold level for a prolonged period of time was required for optimal therapeutic effects.

Xenograft Model of Metastasis

The compounds of this invention are also tested for inhibition of late metastasis using an experimental metastasis model such as that described by Crowley, C. W. et al., Proc. Natl. Acad. Sci. USA 90 5021-5025 (1993)). Late metastasis involves the steps of attachment and extravasation of tumor cells, local invasion, seeding, proliferation and angiogenesis. Human melanoma cells transfected with a reporter gene, preferably the green fluorescent protein (GFP) gene, but as an alternative with a gene encoding the enzymes chloramphenicol acetyl-transferase (CAT), luciferase or LacZ, are inoculated into nude mice. This permits utilization of either of these markers (fluorescence detection of GFP or histochemical colorimetric detection of enzymatic activity) to follow the fate of these cells. Cells are injected, preferably iv, and metastases identified after about 14 days, particularly in the lungs but also in regional lymph nodes, femurs and brain. This mimics the organ tropism of naturally occurring metastases in human melanoma. For example, GFP-expressing melanoma cells (10⁶ cells per mouse) are injected i.v. into the tail veins of nude mice. Animals are treated with a test composition at 100 μg/animal/day given q.d. IP. Single metastatic cells and foci are visualized and quantitated by fluorescence microscopy or light microscopic histochemistry or by grinding the tissue and quantitative colorimetric assay of the detectable label.

Representative mice are subjected to histopathological and immunocytochemical studies to further document the presence of metastases throughout the major organs. Number and size (greatest diameter) of the colonies can be tabulated by digital image analysis, e.g. as described by Fu, Y. S. et al., Anat. Quant. Cytol. Histol. 11:187-195 (1989)).

For determination of colonies, explants of lung, liver, spleen, para-aortic lymph nodes, kidney, adrenal glands and s.c. tissues are washed, minced into pieces of 1-2 mm³ and the pieces pulverized in a Tekman tissue pounder for 5 min. The pulverized contents are filtered through a sieve, incubated in a dissociation medium (MEM supplemented with 10% FCS, 200 U/ml of collagenase type I and 100 μg/ml of DNase type I) for 8 hr at 37° C. with gentle agitation. Thereafter, the resulting cell suspension is washed and resuspended in regular medium (e.g., MEM with 10% FCS supplemented with the selecting antibiotic (G-418 or hygromycin). The explants are fed and the number of clonal outgrowths of tumor cells is determined after fixation with ethanol and staining with an appropriate ligand such as a monoclonal antibody to a tumor cell marker. The number of colonies is counted over an 80-cm² area. If desired, a parallel set of experiments can be conducted wherein clonal outgrowths are not fixed and stained but rather are retrieved fresh with cloning rings and pooled after only a few divisions for other measurements such as secretion of collagenases (by substrate gel electrophoresis) and Matrigel invasion.

Matrigel invasion assays are described herein, though it is possible to use assays described by others (Hendrix, M. J. C. et al., Cancer Lett., 38:137-147 (1987); Albini, A. et al., Cancer Res., 47 3239-3245 (1987); Melchiori, A., Cancer Res. 52:2353-2356 (1992)).

All experiments are performed with groups that preferably have 10 mice. Results are analyzed with standard statistical tests.

Depending on the tumor, i.v. injections of 0.2−10×10⁵ tumor cells 1 week after an s.c. flank injection of an equal number of tumor cells followed by an additional 5-week interval yielded a ratio of hematogenous:spontaneous pulmonary metastases and an overall pulmonary tumor burden that is convenient for evaluation. The model may peroit retrieval of numerous extrapulmonary metastatic clones from spleen, liver, kidneys, adrenal gland, para-aortic lymph nodes and s.c. sites, most of which likely represent spontaneous metastases from the locally growing tumor.

Treatment Procedure

Doses of the test composition are determined as described above using, inter alia, appropriate animal models of the tumor of cancer of interest. A pharmaceutical composition of the present invention is administered. A treatment consists of injecting the subject with 0.001, 1, 100 and 1000 ng of the compound intravenously in 200 ml of normal saline over a one-hour period. Treatments are given 3×/week for a total of 12 treatments. Patients with stable or regressing disease are treated beyond the 12th treatment. Treatment is given on either an outpatient or inpatient basis as needed.

Patient Evaluation

Assessment of response of the tumor to the therapy is made once per week during therapy and 30 days thereafter. Depending on the response to treatment, side effects, and the health status of the patient, treatment is terminated or prolonged from the standard protocol given above. Tumor response criteria are those established by the International Union Against Cancer and are listed below.

| RESPONSE | DEFINITION |
|---|---|
| Complete remission (CR) | Disappearance of all evidence of disease |
| Partial remission (PR) | ≧50% decrease in the product of the |
| | two greatest perpendicular tumor |
| | diameters; no new lesions |
| Less than partial | 25%-50% decrease in tumor size, stable |
| remission (<PR) | for at least 1 month |
| Stable disease | <25% reduction in tumor size; no pro- |
| | gression or new lesions |
| Progression | ≧25% increase in size of any one measured |
| | lesion or appearance of new lesions |
| | despite stabilization or remission of |
| | disease in other measured sites |

The efficacy of the therapy in a patient population is evaluated using conventional statistical methods, including, for example, the Chi Square test or Fisher's exact test. Long-term changes in and short term changes in measurements can be evaluated separately.
Results

One hundred and fifty patients are treated. The results are summarized below. Positive tumor responses (at least partial remission) are observed in over 80% of the patients as follows:

| Response | % |
|---|---|
| PR | 66% |
| <PR | 20% |
| PR + <PR | 86% |

Toxicity:

The incidence of side effects are between 10% and <1% of total treatments and are clinically insignificant.

For a GA derivative compound to be useful in accordance with this invention, it should demonstrate activity at the femtomolar level in at least one of the in vitro, biochemical, or molecular assays described herein and also have potent antitumor activity in vivo.

Having now generally described the invention, the same will be more readily understood through reference to the following examples which are provided by way of illustration, and are not intended to be limiting of the present invention, unless specified.

Having now generally described the invention, the same will be more readily understood through reference to the following examples which are provided by way of illustration, and are not intended to be limiting of the present invention, unless specified.

### EXAMPLES 1-19

### Synthesis and/or Characterization of Geldanamycin and Derivatives

General Methods. Melting points are uncorrected. Infrared spectra were recorded on a Matton Galaxy Series FTIR 3000 spectrophotometer. Ultraviolet-visible spectra were recorded on a Hitachi U-4001 spectrophotometer. ¹H and ¹³C NMR spectra were recorded on Varian Inova-600, UnityPlus-500, VRX-500 or VRX-300 spectrometers. The numbering used in all assignments is based on GA ring system (Sasaki, K et al., J. Am. Chem. Soc. 92:7591 (1970)) unless otherwise indicated). Mass spectra were performed by the MSU Mass Spectrometry Facility. GA and macbecin II were provided by the National Cancer Institutes. Macbecin I was synthesized from macbecin II per published procedure (Muroi, M et al., 1980). Radicicol was obtained commercially (Sigma-Aldrich). Anhydrous solvents were purified using standard methods.

### EXAMPLE 1

### (+)-Geldanamycin (1)

IR (in CH₂Cl₂) (cm¹) 3535, 3421, 3364, 3060, 2989, 2968, 1733, 1690, 1650, 1603, 1500, 1367, 1284, 1262, 1193, 1135, 1098, 1054; ¹H NMR (CDCl₃, 500 MHz, assignment aided by COSY) δ 8.69 (s, 1H) (22-NH), 7.27 (s, 1H) (19-H), 6.92 (bd, J=11.5 Hz, 1H) (3-H), 6.55 (ddd, J=11.5, 11.0, 1.0 Hz, 1H) (4-H), 5.86 (dd, J=11.0, 10.0 Hz, 1H) (5-H), 5.80 (bd, J=9.5 Hz, 1H) (9-H), 5.17 (s, 1H) (7-H), 4.77 (bs, 2H) (7-O₂CNH₂), 4.29 (bd, J=10.0 Hz, 1H) (6-H), 4.10 (s, 3H) (17-OCH₃), 3.51 (ddd, J=9.0, 6.5, 2.0 Hz, 1H) (11-H), 3.37 (ddd, J=9.0, 3.0, 3.0 Hz, 1H) (12-H), 3.34 (s, 3H) (6- or 12-OCH₃), 3.27 (s, 3H) (6- or 12-OCH₃), 3.03 (bd, J=6.5 Hz, 1H) (11-OH), 2.76 (dqd, J=9.5, 7.0, 2.0 Hz, 1H) (10-H), 2.50-2.39 (m, 2H) (15-H and H′), 2.00 (bs, 3H) (2-CH₃), 1.81-1.70 (m, 2H) (13-H and H′), 1.77 (d, J=1.0 Hz, 3H) (8-CH₃), 1.68-1.60 (m, 1H) (14-H), 0.97-0.93 (m, 6H) (10- and 14-CH₃); (Sasaki et al., 1970, supra; Organic Synthesis, Cumulative Volume 4, 433, “Ethyleneimine”). ¹³C NMR (CDCl₃, 125 MHz, assignment of protonated carbons aided by DEPT) δ 185.0 (18-C), 184.1 (21-C), 168.2 (1-C), 157.0 (17-C), 155.9 (7-O₂CNH₂), 138.1 (20-C), 136.4 (5-C), 134.8 (2-C), 133.3 (8-C), 133.1 (9-C), 127.6 (16-C), 127.2 (3-C), 126.3 (4-C), 111.7 (19-C), 81.7 (7-C), 81.3 (12-C), 81.0 (6-C), 72.7 (11-C), 61.7 (17-OCH₃), 57.3 (6- or 12-OCH₃), 56.7 (6- or 12-OCH₃), 34.7 (13-C), 32.7 (15-C), 32.2 (10-C), 27.9 (14-C), 22.9 (14-CH₃), 12.8 (8-CH₃), 12.5 (2-CH₃), 12.4 (10-CH₃). (For ¹³C NMR of GA, see: Johnson, R D et al., J. Am. Chem. Soc. 96:3316 (1974); Johnson, R D et al., J. Am. Chem. Soc. 99:3541 (1977)).

### EXAMPLE 2

### 17-Allylamino-17-demethoxygeldanamycin (4)

(Schnur, R C et al., 1995a, 1995b) (+)-Geldanamycin (5.1 mg, 9.0 μmol) was stirred with allylamine (10.0 μl, 0.13 mmol) in chloroform (1.5 ml) at room temperature. Upon the complete conversion of GA shown by thin layer chromatography (18 hours), the mixture was washed with brine, dried over anhydrous sodium sulfate, and concentrated. Separation by flash column chromatography on silica gel (hexane/ethyl acetate) gave the product as a purple solid (5.3 mg, 99%). IR (KBr) (cm⁻¹) 3464, 3333, 2958, 2929, 2825, 1728, 1691, 1652, 1571, 1485, 1372, 1323, 1189, 1101, 1057; UV (95% EtOH) (nm) 332 (ε=2.0×10⁴); ¹H NMR (CDCl₃, 500 MHz) δ 9.14 (s, 1H), 7.28 (s, 1H), 6.93 (bd, J=11.5 Hz, 1H), 6.56 (bdd, J=11.5, 11.0 Hz, 1H), 6.38 (bt, J=6.0 Hz, 1H), 5.94-5.81 (m, 3H), 5.30-5.24 (m, 2H), 5.17 (s, 1H), 4.82 (bs, 2H), 4.29 (bd, J=10.0 Hz, 1H), 4.21 (bs, 1H), 4.18-4.08 (m, 2H), 3.55 (ddd, J=9.0, 6.5, 2.0 Hz, 1H), 3.43 (ddd, J=9.0, 3.0, 3.0 Hz, 1H), 3.34 (s, 3H), 3.25 (s, 3H), 2.72 (dqd, J=9.5, 7.0, 2.0 Hz, 1H), 2.63 (d, J=14.0 Hz, 1H), 2.34 (dd, J=14.0, 11.0 Hz, 1H), 2.00 (bs, 3H), 1.78 (d, J=1.0 Hz, 3H), 1.78-1.74 (m, 2H), 1.74-1.67 (m, 1H), 0.99-0.95 (m, 6H); ¹³C NMR (CDCl₃, 125 MHz, assignment of protonated carbons aided by DEPT) δ 183.8 (18-C), 180.9 (21-C), 168.4 (1-C), 156.0 (7-O₂CNH₂), 144.6 (17-C), 141.2 (20-C), 135.8 (5-C), 134.9 (2-C), 133.7 (9-C), 132.7 (8-C), 132.5 (3′-C), 126.9 (4-C), 126.5 (3-C), 118.5 (3′-C), 108.8 (19-C), 108.7 (16-C), 81.6 (7-C), 81.4 (12-C), 81.2 (6-C), 72.6 (11-C), 57.1 (6- or 12-OCH₃), 56.7 (6- or 12-OCH₃), 47.8 (1′-C), 35.0 (13-C), 34.3 (15-C), 32.3 (10-C), 28.4 (14-C), 22.9 (14-CH₃), 12.8 (8-CH₃), 12.6 (2-CH₃), 12.3 (10-CH₃); HRMS (FAB) found 586.3120 [M+H]⁺, calcd. 586.3129 for C₃₁H₄₄N₃O₈.

### Hydroquinone Form of (4)

### 17-Allylamino-17-demethoxy-18,21-dihydrogeldanamycin. (DJAAG)

17-Allyamino-17-demethoxygeldanamycin (3.2 mg, 5.5 μmol) was dissolved in ethyl acetate (3.0 ml), then an aqueous solution (2.5 ml) of sodium dithionite (˜85%, 0.50 g, 2.4 mmol) was added. The mixture was stirred at room temperature for 2 hours. Under nitrogen protection, the light yellow organic layer was separated, washed with brine, dried over anhydrous sodium sulfate, and concentrated to give the product as a dark yellow solid (3.0 mg, 93%). ¹H NMR (done in CDCl₃ following exchangeable hydrogen exchange with D₂O—Na₂S₂O₄, 500 MHz) δ 7.66 (bs, 1H), 6.87 (bd, J=11.5 Hz, 1H), 6.39 (bdd, J=11.5, 11.0 Hz, 1H), 6.04-5.96 (ddt, J=16.0, 10.0, 5.5 Hz, 1H), 5.77 (bd, J=9.5 Hz, 1H), 5.68 (bdd, J=11.0, 10.0 Hz, 1H), 5.29 (bd, J=16.0 Hz, 1H), 5.13 (bd, J=10.0 Hz, 1H), 5.01 (s, 1H), 4.30 (bd, J=10.0 Hz, 1H), 3.56 (bdd, J=9.0, 2.0 Hz, 1H), 3.47 (bd, J=5.5 Hz, 2H), 3.37-3.32 (m, 1H), 3.32 (s, 3H), 3.23 (s, 3H), 2.80-2.71 (m, 1H), 2.61-2.51 (m, 1H), 1.90 (bs, 1H), 1.79-1.72 (m, 7H), 1.66-1.61 (m, 1H), 0.96 (d, J=6.5 Hz, 3H), 0.85 (d, J=7.0 Hz, 3H).

### EXAMPLE 3

### 17-(2-Dimethylaminoethyl)amino-17-demethoxygeldanamycin (5)

(Egorin, M J et al., 2002). N,N-Dimethylethylenediamine (6.0 μl, 0.055 mmol) was added to a solution of (+)-geldanamycin (4.3 mg, 7.7 μmol) in chloroform (1.0 ml). The mixture was stirred at room temperature. Upon the complete conversion of GA shown by thin layer chromatography (4 hours), the mixture was washed with 0.5% aqueous sodium hydroxide solution and brine, dried over anhydrous sodium sulfate, and concentrated. Separation by flash column chromatography on silica gel (ethyl acetate/methanol) gave the product as a purple solid (4.5 mg, 95%). IR (KBr) (cm⁻¹) 3462, 3329, 2932, 2871, 2824, 2774, 1733, 1690, 1653, 1565, 1485, 1373, 1321, 1253, 1188, 1100, 1055; UV (95% EtOH) (nm) 332 (ε=1.7×10⁴); ¹H NMR (CDCl₃, 500 MHz) δ 9.18 (s, 1H), 7.24 (s, 1H), 7.04 (bt, J=5.0 Hz, 1H), 6.94 (bd, J=11.5 Hz, 1H), 6.57 (bdd, J=11.5, 11.0 Hz, 1H), 5.90 (bd, J=9.5 Hz, 1H), 5.84 (dd, J=11.0, 10.0 Hz, 1H), 5.17 (s, 1H), 4.75 (bs, 2H), 4.42 (bs, 1H), 4.29 (bd, J=10.0 Hz, 1H), 3.70-3.42 (m, 3H), 3.57 (bdd, J=9.0, 6.5 Hz, 1H), 3.34 (s, 3H), 3.25 (s, 3H), 2.72 (dqd, J=9.5, 7.0, 2.0 Hz, 1H), 2.67 (d, J=14.0 Hz, 1H), 2.55 (t, J=5.5 Hz, 2H), 2.38 (dd, J=14.0, 11.0 Hz, 1H), 2.25 (s, 6H), 2.01 (bs, 3H), 1.83-1.68 (m, 3H), 1.78 (bs, 3H), 0.98 (d, J=7.0 Hz, 3H), 0.95 (d, J=6.5 Hz, 3H); MS (FAB) found 617 [M+H]⁺.

### EXAMPLE 4

### 17-Amino-17-demethoxygeldanamycin (6)

(Schnur et al., 1995b; Li, L H et al., 1977; Sasaki, K et al., 1979). Concentrated aqueous solution of ammonia (28%, 0.70 ml, 0.010 mol) was added to a solution of (+)-geldanamycin (5.0 mg, 9.0 μmol) in acetonitrile (5.0 ml) at room temperature. The yellow solution turned slowly dark red. Upon the complete conversion of GA shown by thin layer chromatography (5 hours), the mixture was partitioned between ethyl acetate and brine. The organic phase was washed with brine, dried over anhydrous sodium sulfate, and concentrated. Separation of the solid residue by flash column chromatography on silica gel (hexane/ethyl acetate) gave the product as a dark red solid (4.6 mg, 95%). IR (KBr) (cm⁻¹) 3452, 3339, 2957, 2931, 2825, 1721, 1692, 1617, 1591, 1495, 1374, 1323, 1250, 1190, 1133, 1101, 1055; UV (95% EtOH) (nm) 328 (E=2.0×10⁴); ¹H NMR (CDCl₃, 500 MHz) δ 9.08 (s, 1H), 7.26 (s, 1H), 6.95 (bd, J=11.5 Hz, 1H), 6.56 (bdd, J=11.5, 11.0 Hz, 1H), 5.89-5.82 (m, 2H), 5.37 (bs, 2H), 5.17 (s, 1H), 4.73 (bs, 2H), 4.29 (bd, J=10.0 Hz, 1H), 3.98 (bs, 1H), 3.59 (ddd, J=9.0, 6.5, 2.0 Hz, 1H), 3.42 (ddd, J=9.0, 3.0, 3.0 Hz, 1H), 3.34 (s, 3H), 3.25 (s, 3H), 2.75 (dqd, J=9.5, 7.0, 2.0 Hz, 1H), 2.65 (d, J=14.0 Hz, 1H), 2.01 (bs, 3H), 1.97-1.75 (m, 4H), 1.79 (d, J=1.0 Hz, 3H), 0.99-0.97 (m, 6H); ¹³C NMR (CDCl₃, 125 MHz) δ 183.1, 180.4, 167.9, 156.1, 146.0, 140.4, 135.8, 135.0, 134.0, 133.0, 126.9, 126.6, 110.3, 108.6, 81.9, 81.2, 81.1, 72.2, 57.1, 56.8, 35.0, 34.7, 32.2, 28.7, 23.8, 12.8, 12.5, 12.2; HRMS (FAB) found 546.2818 [M+H]⁺, calcd. 546.2816 for C₂₈H₄₀N₃O₈.

### EXAMPLE 5

### 17-(2-Chloroethyl)amino-17-demethoxygeldanamycin (7)

(Sasaki et al., supra). Sodium hydroxide aqueous solution (2.80 M, 0.75 ml, 2.1 mmol) was added to a mixture of (+)-geldanamycin (11.7 mg, 0.021 mmol) and 2-chloroethylamine hydrochloride (242 mg, 2.1 mmol) in acetonitrile (3.0 ml). The mixture was stirred at room temperature. Upon the complete conversion of GA shown by thin layer chromatography (20 hours), the mixture was partitioned between ethyl acetate and brine. The organic phase was washed with brine, dried over anhydrous sodium sulfate, and concentrated. Separation by flash column chromatography on silica gel (hexane/ethyl acetate) gave the product as a purple solid (12.0 mg, 95%). IR (KBr) (cm⁻¹) 3334, 2938, 2874, 2822, 1733, 1696, 1653, 1577, 1489, 1375, 1325, 1274, 1190, 1136, 1101, 1060; UV (95% EtOH) (nm) 332 (ε=1.9×10⁴); ¹H NMR (CDCl₃, 500 MHz) δ 9.09 (s, 1H), 7.29 (s, 1H), 6.94 (bd, J=11.5 Hz, 1H), 6.56 (ddd, J=11.5, 11.0, 1.0 Hz, 1H), 6.35 (bt, J=5.0 Hz, 1H), 5.87 (bd, J=9.5 Hz, 1H), 5.85 (bdd, J=11.0, 10.0 Hz, 1H), 5.18 (s, 1H), 4.72 (bs, 2H), 4.30 (bd, J=10.0 Hz, 1H), 4.03 (bs, 1H), 3.94-3.83 (m, 2H), 3.75-3.67 (m, 2H), 3.56 (ddd, J=9.0, 6.5, 2.0 Hz, 1H), 3.43 (ddd, J=9.0, 3.0, 3.0 Hz, 1H), 3.35 (s, 3H), 3.26 (s, 3H), 2.73 (dqd, J=9.5, 7.0, 2.0 Hz, 1H), 2.70 (d, J=14.0 Hz, 1H), 2.24 (dd, J=14.0, 11.0 Hz, 1H), 2.01 (bs, 3H), 1.78 (d, J=1.0 Hz, 3H), 1.80-1.75 (m, 2H), 1.75-1.68 (m, 1H), 1.00-0.96 (m, 6H); ¹³C NMR (CDCl₃, 125 MHz) [symbol] 183.8, 181.2, 168.3, 155.9, 144.7, 140.8, 135.9, 135.0, 133.6, 132.9, 127.0, 126.5, 110.0, 109.1, 81.6, 81.4, 81.2, 72.7, 57.1, 56.7, 46.9, 42.7, 35.1, 34.4, 32.4, 28.8, 23.0, 12.8, 12.6, 12.5; MS (FAB) found 608 [M+H]⁺.

### EXAMPLE 6

### 17-(2-Fluoroethyl)amino-17-demethoxygeldanamycin. (8)

(Schnur et al., 1995b). Sodium hydroxide aqueous solution (1.10 M, 0.53 ml, 0.58 mmol) was added to a mixture of (+)-geldanamycin (5.5 mg, 9.8 μmol) and 2-fluoroethylamine hydrochloride (65 mg, 0.59 mmol) in acetonitrile (1.0 ml). The mixture was stirred at room temperature. Upon the complete conversion of GA shown by thin layer chromatography (12 hours), the mixture was partitioned between ethyl acetate and brine. The organic phase was washed with brine, dried over anhydrous sodium sulfate, and concentrated. Separation by flash column chromatography on silica gel (hexane/ethyl acetate) gave the product as a purple solid (5.7 mg, 98%). IR (KBr) (cm⁻¹) 3465, 3330, 2954, 2927, 2873, 1728, 1691, 1653, 1576, 1487, 1375, 1323, 1255, 1190, 1103, 1051; UV (95% EtOH) (nm) 332 (ε=1.7×10⁴); ¹H NMR (CDCl₃, 500 MHz) δ 9.10 (s, 1H), 7.29 (s, 1H), 6.94 (bd, J=11.5 Hz, 1H), 6.57 (bdd, J=11.5, 11.0 Hz, 1H), 6.36 (bt, J=5.0 Hz, 1H), 5.88-5.83 (m, 2H), 5.18 (s, 1H), 4.75 (bs, 2H), 4.69-4.57 (m, 2H), 4.30 (bd, J=10.0 Hz, 1H), 3.94-3.76 (m, 2H), 3.56 (bd, J=9.0 Hz, 1H), 3.43 (ddd, J=9.0, 3.0, 3.0 Hz, 1H), 3.35 (s, 3H), 3.26 (s, 3H), 2.73 (dqd, J=9.5, 7.0, 2.0 Hz, 1H), 2.70 (d, J=14.0 Hz, 1H), 2.30 (dd, J=14.0, 11.0 Hz, 1H), 2.01 (bs, 3H), 1.80-1.76 (m, 2H), 1.78 (d, J=1.0 Hz, 3H), 1.75-1.68 (m, 1H), 0.99 (d, J=7.0 Hz, 3H), 0.97 (d, J=6.5 Hz, 3H); ¹³C NMR (CDCl₃, 125 MHz) δ 183.8, 181.2, 168.4, 156.0, 144.9, 140.9, 135.9, 135.0, 133.6, 132.8, 127.0, 126.5, 109.7, 109.1, 81.6, 81.5 (d, J=170 Hz), 81.4, 81.2, 72.6, 57.2, 56.7, 46.0 (d, J=20 Hz), 35.1, 34.3, 32.4, 28.8, 23.0, 12.8, 12.6, 12.5; HRMS (FAB) found 591.2952 [M]⁺, calcd. 591.2956 for C₃₀H₄₂FN₃O₈.

### EXAMPLE 7

### 17-(2-Acetylaminoethyl)amino-17-demethoxygeldanamycin (9)

(Schnur et al., 1995b). N-Acetylethylenediamine (90%, 10.0 μl, 0.094 mmol) was added to a solution of (+)-geldanamycin (5.0 mg, 8.9 μmol) in chloroform (1.0 ml) at room temperature. Upon the complete conversion of GA shown by thin layer chromatography (10 hours), the mixture was washed with distilled water, dried over anhydrous sodium sulfate, and concentrated. Separation by flash column chromatography on silica gel (ethyl acetate) gave the product as a purple solid (4.5 mg, 80%). IR (KBr) (cm⁻¹) 3449, 3338, 2932, 2881, 2824, 1718, 1685, 1654, 1569, 1487, 1374, 1323, 1269, 1189, 1102, 1057; ¹H NMR (CDCl₃, 500 MHz) δ 9.12 (s, 1H), 7.23 (s, 1H), 6.94 (bd, J=11.5 Hz, 1H), 6.63 (bt, J=5.0 Hz, 1H), 6.56 (bdd, J=11.5, 11.0 Hz, 1H), 5.88 (bd, J=9.5 Hz, 1H), 5.84 (dd, J=11.0, 10.0 Hz, 1H), 5.80 (bt, J=6.0 Hz, 1H), 5.17 (s, 1H), 4.72 (bs, 2H), 4.29 (bd, J=10.0 Hz, 1H), 4.17 (bs, 1H), 3.77-3.62 (m, 2H), 3.58-3.46 (m, 3H), 3.42 (ddd, J=9.0, 3.0, 3.0 Hz, 1H), 3.34 (s, 3H), 3.25 (s, 3H), 2.73 (dqd, J=9.5, 7.0, 2.0 Hz, 1H), 2.64 (d, J=14.0 Hz, 1H), 2.33 (dd, J=14.0, 11.0 Hz, 1H), 2.01 (s, 3H), 2.00 (d, J=1.0 Hz, 3H), 1.80-1.76 (m, 2H), 1.78 (d, J=1.0 Hz, 3H), 1.74-1.67 (m, 1H), 0.98 (d, J=7.0 Hz, 3H), 0.95 (d, J=6.5 Hz, 3H); HRMS (FAB) found 631.3344 [M+H]⁺, calcd. 631.3343 for C₃₂H₄₇N₄O₉.

### EXAMPLE 8

### 17-(6-Acetylamino-1-hexyl)amino-17-demethoxygeldanamycin (10)

A solution of (+)-geldanamycin (5.7 mg, 0.010 mmol) and N-(6-aminohexyl)acetamide (5.5 mg, 0.035 mmol) in chloroform was stirred at room temperature. Upon the complete conversion of GA shown by thin layer chromatography (20 hours), the mixture was washed with distilled water, dried over anhydrous sodium sulfate, and concentrated. Separation by flash column chromatography on silica gel (ethyl acetate) gave the product as a purple solid (5.7 mg, 82%). IR (KBr) (cm⁻¹) 3445, 3323, 3202, 2931, 2865, 2824, 1723, 1687, 1653, 1562, 1486, 1371, 1322, 1256, 1188, 1135, 1106; UV (95% EtOH) (nm) 333 (ε=1.2×10⁴); ¹H NMR (CDCl₃, 500 MHz, assignment aided by COSY) δ 9.17 (bs, 1H), 7.26 (s, 1H), 6.94 (bd, J=11.5 Hz, 1H), 6.57 (bdd, J=11.5, 11.0 Hz, 1H), 6.26 (bt, J=5.0 Hz, 1H), 5.89 (bd, J=9.5 Hz, 1H), 5.85 (dd, J=11.0, 10.0 Hz, 1H), 5.42 (bs, 1H), 5.18 (s, 1H), 4.73 (bs, 2H), 4.31 (bs, 1H), 4.29 (bd, J=10.0 Hz, 1H), 3.59-3.39 (m, 4H), 3.35 (s, 3H), 3.27-3.19 (m, 2H), 3.25 (s, 3H), 2.74 (dqd, J=9.5, 7.0, 2.0 Hz, 1H), 2.66 (d, J=14.0 Hz, 1H), 2.39 (dd, J=14.0, 11.0 Hz, 1H), 2.01 (bs, 3H), 1.96 (s, 3H), 1.80-1.75 (m, 2H), 1.78 (d, J=1.0 Hz, 3H), 1.73-1.62 (m, 3H), 1.55-1.47 (m, 2H), 1.46-1.33 (m, 4H), 0.99 (d, J=7.0 Hz, 3H), 0.95 (d, J=6.5 Hz, 3H); ¹³C NMR (CDCl₃, 125 MHz) δ 183.9, 180.7, 170.0, 168.4, 156.0, 144.9, 141.5, 135.9, 135.0, 133.8, 132.8, 127.0, 126.6, 108.7, 108.4, 81.7, 81.5, 81.2, 72.7, 57.2, 56.7, 45.8, 39.4, 35.1, 34.4, 32.4, 29.7, 29.6, 28.6, 26.5, 26.4, 23.4, 22.9, 12.8, 12.6, 12.4; HRMS (FAB) found 687.3967 [M+H]⁺, calcd. 687.3969 for C₃₆H₅₅N₄O₉.

### EXAMPLE 9

### (+)-Biotin 17-(6-aminohexyl)amino-17-demethoxygeldanamycin amide (11)

1,6-Diaminohexane (10.0 mg, 0.086 mmol) was added to a solution of (+)-geldanamycin (5.0 mg, 8.9 μmol) in chloroform (1.0 ml) at room temperature. Upon the complete conversion of GA shown by thin layer chromatography (20 hours), the mixture was washed with 0.5% aqueous sodium hydroxide solution and brine, dried over potassium carbonate and concentrated. The resulted dark purple solid was then stirred overnight with (+)-biotin N-hydroxysuccinimide ester (3.0 mg, 8.8 μmol) in DMF (1.0 ml). Removal of the solvent and separation by flash column chromatography on silica gel (ethyl acetate/methanol) gave the product as a purple solid (6.5 mg, 85%). IR (KBr) (cm⁻¹) 3327, 2931, 2864, 1709, 1651, 1562, 1485, 1371, 1325, 1255, 1099, 731; ¹H NMR (CDCl₃, 500 MHz) [symbol] 9.19 (s, 1H), 7.24 (s, 1H), 6.94 (bd, J=11.5 Hz, 1H), 6.56 (bdd, J=11.5, 11.0 Hz, 1H), 6.28 (bt, J=5.0 Hz, 1H), 5.87 (bd, J=9.5 Hz, 1H), 5.84 (dd, J=11.0, 10.0 Hz, 1H), 5.88-5.77 (m, 2H), 5.17 (s, 1H), 5.15 (bs, 1H), 4.87 (bs, 2H), 4.50 (dd, J=7.5, 5.0 Hz, 1H), 4.32-4.29 (m, 2H), 4.23 (bs, 1H), 3.58-3.41 (m, 4H), 3.34 (s, 3H), 3.26 (s, 3H), 3.24-3.20 (m, 2H), 3.17-3.12 (m, 1H), 2.91 (dd, J=13.0, 5.0 Hz, 1H), 2.75-2.69 (m, 2H), 2.66 (d, J=14.0 Hz, 1H), 2.38 (dd, J=14.0, 11.0 Hz, 1H), 2.21-2.15 (m, 2H), 2.01 (bs, 3H), 1.78 (d, J=1.0 Hz, 3H), 1.78-1.32 (m, 17H), 0.98 (d, J=7.0 Hz, 3H), 0.95 (d, J=6.5 Hz, 3H); ^{[14]}HRMS (FAB) found 871.4619 [M+H]⁺, calcd. 871.4592 for C₄₄H₆₇N₆O₁₀S.

### EXAMPLE 10

### 17-[2-[2-(2-Acetylaminoethoxy)ethoxy]ethyl]amino-17-demethoxygeldanamycin (12)

A mixture of 2,2′-(ethylenedioxy)bis(ethylamine) (56.0 μl, 0.38 mmol), acetic anhydride (46.0 μl, 0.48 mmol) and triethylamine (73.2 μl, 0.52 mmol) in chloroform (1.0 ml) was stirred for 1 hour at room temperature, then concentrated to dryness under high vacuum. The colorless solid residue was then stirred with (+)-geldanamycin (4.0 mg, 7.1 μmol) in chloroform (1.0 ml). Upon the complete conversion of GA shown by thin layer chromatography (20 hours), the mixture was washed with distilled water, dried over anhydrous sodium sulfate, and concentrated. Separation by flash column chromatography on silica gel (ethyl acetate/methanol) gave the desired product as a purple solid (1.1 mg, 21%). IR (KBr) (cm⁻¹) 3446, 3336, 2960, 2929, 2877, 1727, 1689, 1655, 1566, 1487, 1375, 1325, 1261, 1190, 1103, 1057; ¹H NMR (CDCl₃, 300 MHz) δ 9.17 (s, 1H), 7.25 (s, 1H), 6.94 (bd, J=11.5 Hz, 1H), 6.78 (bt, J=5.0 Hz, 1H), 6.57 (bdd, J=11.5, 11.0 Hz, 1H), 6.36 (bs, 1H), 5.89 (bd, J=9.5 Hz, 1H), 5.85 (dd, J=11.0, 10.0 Hz, 1H), 5.18 (s, 1H), 4.74 (bs, 2H), 4.29 (bd, J=10.0 Hz, 1H), 4.26 (bs, 1H), 3.78-3.40 (m, 14H), 3.35 (s, 3H), 3.25 (s, 3H), 2.78-2.64 (m, 2H), 2.39 (dd, J=14.0, 11.0 Hz, 1H), 2.01 (bs, 3H), 1.98 (s, 3H), 1.78-1.67 (m, 3H), 1.78 (d, J=1.0 Hz, 3H), 0.99-0.94 (m, 6H); HRMS (FAB) found 719.3864 [M+H]⁺, calcd. 719.3867 for C₃₆H₅₅N₄O₁₁.

### EXAMPLE 11

### 17-Carboxymethylamino-17-demethoxygeldanamycin (13)

(+)-Geldanamycin (3.1 mg, 5.5 μmol) was stirred at room temperature with glycine sodium salt (10.7 mg, 0.11 mmol) in a mixture of ethanol (1.2 ml) and water (0.3 ml). Upon the complete conversion of GA shown by thin layer chromatography (3 hours), the purple mixture was acidified with diluted hydrochloric acid and partitioned between chloroform and distilled water. The organic phase was dried over anhydrous sodium sulfate and concentrated. Separation by flash column chromatography on silica gel (ethyl acetate/methanol) gave the product as a purple solid (3.2 mg, 96%). IR (KBr) (cm⁻¹) 3446, 3305, 2929, 2875, 1734, 1693, 1655, 1618, 1574, 1485, 1394, 1319, 1267, 1139, 1072; ¹H NMR (CDCl₃, 500 MHz) δ 8.91 (s, 1H), 7.25 (s, 1H), 6.83 (bs, 1H), 6.80 (bd, J=11.5 Hz, 1H), 6.60 (bdd, J=11.5, 11.0 Hz, 1H), 5.86-5.80 (m, 2H), 5.16 (s, 1H), 4.95 (bs, 2H), 4.33-4.21 (m, 2H), 4.27 (bd, J=10.0 Hz, 1H), 3.54 (dd, J=9.0, 2.0 Hz, 1H), 3.42 (ddd, J=9.0, 3.0, 3.0 Hz, 1H), 3.33 (s, 3H), 3.25 (s, 3H), 2.70 (dqd, J=9.5, 7.0, 2.0 Hz, 1H), 2.59 (d, J=14.0 Hz, 1H), 2.27 (dd, J=14.0, 11.0 Hz, 1H), 2.07 (bs, 3H), 1.80-1.75 (m, 2H), 1.62-1.54 (m, 1H), 1.77 (bs, 3H), 0.98 (d, J=7.0 Hz, 3H), 0.91 (d, J=6.5 Hz, 3H); HRMS (FAB) found 604.2867 [M+H]⁺, calcd. 604.2870 for C₃₀H₄₂N₃O₁₀.

### EXAMPLE 12

### 17-(1-Azetidinyl)-17-demethoxygeldanamycin (14)

(Schnur, R C et al., 1994). Azetidine (4.0 μl, 0.059 mmol) was added to a solution of (+)-geldanamycin (7.5 mg, 0.013 mmol) in dichloromethane (1.5 ml) with stirring. Upon the complete conversion of GA shown by thin layer chromatography (40 minutes), the mixture was washed with brine, dried over anhydrous sodium sulfate, and concentrated. Separation by flash column chromatography on silica gel (hexane/ethyl acetate) gave the product as a deep purple solid (7.7 mg, 98%). IR (in CH₂Cl₂) (cm⁻¹) 3422, 3075, 3049, 2986, 1733, 1686, 1651, 1605, 1540, 1486, 1420, 1375, 1283, 1260, 1103, 1047; ¹H NMR (CDCl₃, 500 MHz) δ 9.16 (s, 1H), 7.10 (s, 1H), 6.92 (bd, J=11.5 Hz, 1H), 6.56 (bdd, J=11.5, 11.0 Hz, 1H), 5.92 (bd, J=9.5 Hz, 1H), 5.82 (dd, J=1.0, 10.0 Hz, 1H), 5.15 (s, 1H), 4.79 (bs, 2H), 4.72-4.58 (m, 4H), 4.28 (bd, J=10.0 Hz, 1H), 3.54 (bd, J=9.0 Hz, 1H), 3.43 (ddd, J=9.0, 3.0, 3.0 Hz, 1H), 3.34 (s, 3H), 3.24 (s, 3H), 2.71 (dqd, J=9.5, 7.0, 2.0 Hz, 1H), 2.59 (d, J=14.0 Hz, 1H), 2.42 (quintet, J=8.0 Hz, 2H), 2.23 (dd, J=14.0, 11.0 Hz, 1H), 2.00 (bs, 3H), 1.78 (bs, 3H), 1.77-1.73 (m, 2H), 1.69-1.62 (m, 1H), 0.97 (d, J=7.0 Hz, 3H), 0.94 (d, J=6.5 Hz, 3H); ¹³C NMR (CDCl₃, 125 MHz, assignment of protonated carbons aided by DEPT and HMQC) δ 185.8 (18-C), 178.4 (21-C), 168.4 (1-C), 156.0 (7-O₂CNH₂), 145.9 (17-C), 140.5 (20-C), 135.5 (5-C), 135.1 (2-C), 134.0 (9-C), 132.6 (8-C), 126.7 (4-C), 126.6 (3-C), 109.6 (19-C), 109.2 (16-C), 81.8 (7-C), 81.6 (12-C), 81.3 (6-C), 72.5 (11-C), 58.9 (1′- and 3′-C), 57.1 (6- or 12-OCH₃), 56.7 (6- or 12-OCH₃), 35.1 (13-C), 34.1 (15-C), 32.3 (10-C), 28.1 (14-C), 22.9 (14-CH₃), 18.4 (2′-C), 12.7 (8-CH₃), 12.6 (2-CH₃), 12.2 (10-CH₃); MS (FAB) found 586 [M+H]⁺.

### EXAMPLE 13

### 17-(1-Aziridinyl)-17-demethoxygeldanamycin (15)

Aziridine (Allen, C F H et al., 1963) (0.30 ml, 5.80 mmol) was added to a solution of (+)-geldanamycin (5.8 mg, 0.010 mmol) in dichloromethane (2.0 ml). The mixture was stirred at room temperature. Upon the complete conversion of GA shown by thin layer chromatography (25 minutes), the mixture was washed with brine, dried over anhydrous sodium sulfate, and concentrated. Separation by flash column chromatography on silica gel (hexane/ethyl acetate) gave the product as an orange solid (5.6 mg, 95%). IR (KBr) (cm⁻¹) 3438, 3338, 3192, 2925, 2827, 1736, 1701, 1687, 1644, 1585, 1517, 1457, 1367, 1272, 1192, 1112; ¹H NMR (CDCl₃, 500 MHz) [symbol] 8.77 (s, 1H) (22—NH), 7.27 (s, 1H) (19-H), 6.91 (bd, J=11.5 Hz, 1H), 6.55 (bdd, J=11.5, 11.0 Hz, 1H), 5.86-5.80 (m, 2H), 5.17 (s, 1H), 4.80 (bs, 2H), 4.30 (bd, J=10.0 Hz, 1H), 3.52 (ddd, J=9.0, 6.5, 2.0 Hz, 1H), 3.42-3.37 (m, 2H), 3.34 (s, 3H), 3.27 (s, 3H), 2.73 (dqd, J=9.5, 7.0, 2.0 Hz, 1H), 2.57 (d, J=14.0 Hz, 1H), 2.50 (dd, J=14.0, 11.0 Hz, 1H), 2.44-2.33 (m, 4H), 2.00 (bs, 3H), 1.80-1.76 (m, 2H), 1.77 (bs, 3H), 1.75-1.69 (m, 1H), 0.99-0.96 (m, 6H); 13 NMR (CDCl₃, 125 MHz, assignment of protonated carbons aided by DEPT) δ 183.8 (18-C), 183.2 (21-C), 168.3 (1-C), 156.0 (7-O₂CNH₂), 152.7 (17-C), 138.8 (20-C), 136.1 (5-C), 134.9 (2-C), 133.3 (9-C), 133.1 (8-C), 127.0 (4-C), 126.4 (3-C), 125.4 (16-C), 111.6 (19-C), 81.6 (7-C), 81.1 (12-C), 81.1 (6-C), 72.7 (11-C), 57.2 (6- or 12-OCH₃), 56.7 (6- or 12-OCH₃), 35.1 (13-C), 33.6 (15-C), 32.3 (10-C), 29.2 (17-NCH₂), 28.9 (14-C), 23.3 (14-CH₃), 12.9 (8-CH₃), 12.5 (2-CH₃), 12.4 (10-CH₃); HRMS (FAB) found 572.2968 [M+H]⁺, calcd. 572.2926 for C₃₀H₄₂N₃O₈.

### EXAMPLE 14

### 5′-Bromogeldanoxazinone (16)

3-bromo-4-nitrophenol and 3-bromo-6-nitrophenol. 3.8 ml of fuming nitric acid (89 mmole) in 12 ml glacial acetic acid was added over 35 minutes to a solution of 15.2 grams (87.9 mmole) of 3-bromophenol in 60 ml of glacial acetic acid in a flask with a surrounding ice bath. The reaction was stirred at room temperature for an additional 30 minutes and the reaction was then poured on ice. This was then concentrated in vacuo. Medium pressure chromatography on silica gel (1:2 ethyl acetate:hexanes as eluent) allowed separation of products 3-bromo-4-nitrophenol (3.47 grams, 15.9 mmole, 18% yield); m.p. 130-131° C. following recrystallization from ether/hexanes (reported m.p. 130 131° C. (Wright, C et al, 1987) and 131° C. (Hodgson, H H et al, 1926); ¹H NMR (DMSO-d6, 500 MHz) δ 7.99 (d, 1H, J=9 Hz), 7.18 (d, 1H, J=3 Hz), 6.91 (dd, 1H, J=9, 3 Hz,); and 3-bromo-6-nitrophenol (1.94 grams, 8.90 mmole, 10% yield, following recrystallization from ether/hexanes); m.p. 41.5-42.5° C. (reported m.p. 42-45° C. (Hanzlik, R P et al., 1990) and 42° C. (Hodson et al.,); ¹H NMR (CDCl₃, 500 MHz) δ 10.60 (s, 1H), 7.95 (d, 1H, J=9 Hz), 7.35 (d, 1H, J=2 Hz), 7.11 (dd, 1H, J=9, 2 Hz,); ¹³C NMR (CDCl₃; assignments aided by HMQC) δ 122.9 (C-2), 123.8 (C-4), 126.0 (C-5), 132.2 (C-3), 132.7 (C-6), 155.2 (C-1); IR (KBr) 3450 (broad), 1612, 1578, 1527, 1475, 1311, 1235, 1186, 900 cm⁻¹).

2-Amino-5-bromophenol. 3-Bromo-6-nitrophenol (0.292 gms, 1.34 mmole) was stirred in an 0.5% aqueous sodium hydroxide solution (30 mL). Sodium hydrosulphite (2.00 gms of 85%, 9.76 mmole) was added to the reaction flask and this was stirred at room temperature for 15 minutes. The reaction flask was then acidified with diluted hydrochloric acid until a pH of 5 was obtained. The reaction was then extracted three times with 40 mL portions of diethyl ether, the combined organic layers dried over anhydrous sodium sulfate, and concentrated to provide crude 2-amino-5-bromophenol (0.533 gms, m.p. 99.5-100.5° C.), which was recrystallized from ethyl ether/hexanes to provide the pure product (0.151 gms, 0.80 mmole, 60% yield; m.p. 125-127° C. (decompose) (reported m.p. 149.5-150.5° C. (Boyland, E et al., 1954); ¹H NMR (CD₃CN, 500 MHz) δ 7.08 (bs, 1H), 6.82 (d, 1H, J=2 Hz), 6.78 (dd, 1H, J=8, 2 Hz), 6.56 (d, 1H, J=8 Hz), 4.03 (bs, 2H); IR (KBr) 3496 (broad), 3377, 3298, 1598, 1502, 1431, 1269, 1210, 916, 877 cm⁻¹)

5′-Bromogeldanoxazinone (16) (Webb et al., supia; Rinehart, K L et al., 1977). A mixture of (+)-geldanamycin (21.8 mg, 0.039 mmol) and 2-amino-5-bromophenol (14.6 mg, 0.078 mmol) in glacial acetic acid (2.0 ml) was stirred at 78° C. under nitrogen for 19 hours, then cooled and concentrated. Separation of the deep orange residue by flash chromatography on silica gel (hexane/ethyl acetate) gave a crude product contaminated with unreacted (+)-geldanamycin. This was then dissolved in chloroform and subjected to preparative HPLC separation (Waters Nova-Pak Silica 6 μm×7.8×300 mm column, 2.0 ml/min CHCl₃/EtOAc 2:3) to afford the product as a bright orange powder (16.4 mg, 60% yield); mp 274-278° C. (decompose) (lit. mp 275-278° C.) (Rinehart, supra). IR (KBr) (cm¹) 3442, 3342, 3209, 2954, 2926, 2878, 1734, 1700, 1615, 1583, 1507, 1384, 1314, 1192, 1111, 1061 (lit. 1605, 1585, 1505) (Rinheart, supra); ¹H NMR (CDCl₃, 500 MHz) [symbol] 9.13 (bs, 1H), 8.33 (s, 1H), 7.73 (d, J=8.5 Hz, 1H), 7.60 (d, J=2.0 Hz, 1H), 7.53 (dd, J=8.5, 2.0 Hz, 1H), 7.03 (bd, J=11.5 Hz, 1H), 6.60 (bdd, J=11.5, 11.0 Hz, 1H), 5.96 (bd, J=9.5 Hz, 1H), 5.86 (dd, J=11.0, 10.0 Hz, 1H), 5.21 (s, 1H), 4.72 (bs, 2H), 4.35 (bd, J=10.0 Hz, 1H), 4.25 (bs, 1H), 3.64 (bdd, J=9.0, 6.5 Hz, 1H), 3.46 (ddd, J=9.0, 3.0, 3.0 Hz, 1H), 3.37 (s, 3H), 3.27 (s, 3H), 2.82-2.71 (m, 3H), 2.08 (bs, 3H), 1.98-1.86 (m, 2H), 1.85-1.77 (m, 1H), 1.81 (d, J=1.0 Hz, 3H), 1.01 (d, J=7.0 Hz, 3H), 0.99 (d, J=6.5 Hz, 3H); ¹³C NMR (CDCl₃, 125 MHz) 180.7, 168.4, 156.0, 148.5, 145.0, 143.5, 136.8, 135.5, 135.3, 133.9, 133.0, 132.9, 130.9, 129.1, 126.7, 125.2, 119.3, 117.5, 112.9, 81.9, 81.3, 81.2, 72.2, 57.1, 56.8, 35.3, 33.1, 32.2, 29.4, 27.6, 23.3, 12.8, 12.7, 12.1; HRMS (FAB) found 698.2080 [M+H]⁺, calcd. 698.2077 for C₃₄H₄₁BrN₃O₈.

### EXAMPLE 15

### 5′-Iodoleldanoxazinone (17)

3-iodo-4-nitrophenol and 3-iodo-6-nitrophenol. 3.0 ml of fuming nitric acid (75 mmole) in 12 ml glacial acetic acid was added over 25 minutes to a solution of 15.03 grams (68.3 mmole) of 3-iodophenol in 60 ml glacial acetic acid in a flask with a surrounding ice bath. The reaction was stirred at room temperature for an additional 30 minutes and the reaction was then poured on ice. This was then concentrated in vacuo, taken up with 150 ml water and extracted with two portions of 300 ml methylene chloride, and the combined methylene chloride layers dried over anhydrous magnesium sulfate and evaporated to give 17 grams organic residue. Medium pressure chromatography on silica gel (1:2 ethyl acetate:hexanes as eluent) allowed separation of products 3-iodo-4-nitrophenol (6.93 grams, 26.1 mmole, 38% yield); m.p. 121-123° C.; ¹H NMR (CDCl₃, 300 MHz) δ 7.98 (d, 1H, J=9 Hz), 7.54 (d, 1H, J=3 Hz), 6.92 (dd, 1H, J=9, 3 Hz), 5.54 (bs, 1H); IR (KBr) 3150 (broad), 1600, 1580, 1512, 1404, 1336, 1298, 1212, 1121, 1023, 870 cm⁻¹; and 3-iodo-6-nitrophenol (3.07 grams, 11.6 mmole, 17% yield; m.p. 92-94° C. following recrystallization from methylene chloride/hexanes (reported m.p. 96° C. (Hodgson, H H et al., 1927); ¹H NMR (CDCl₃, 300 MHz) δ 10.53 (s, 1H), 7.76 (d, 1H, J=9.0 Hz), 7.59 (d, 1H, J=2.0 Hz), 7.33 (dd, 1H, J=9.0, 2.0 Hz); ¹³C NMR (CDCl₃; assignments aided by HMQC) □ 105.2 (C-3), 125.6 (C-5), 129.2 (C-2), 129.7 (C-4), 133.4 (C-6), 154.6 (C-1); IR (KBr) 3430 (broad), 1604, 1571, 1518, 1463, 1317, 1225, 1172, 1055, 888 cm⁻¹; Anal. Calcd for C₆H₄INO₃: C, 27.19; H, 1.52; N, 5.29. Found: C, 27.36; H, 1.57; N, 5.15.).

2-Amino-5-iodophenol. 3-Iodo-6-nitrophenol (0.993 gms, 3.75 mmole) was stirred in an aqueous sodium hydroxide solution (0.233 gm NaOH in 100 mL water). Sodium hydrosulphite (4.62 gms of 85%, 22.6 mmole) was added to the reaction flask and this was stirred at room temperature for 40 minutes. The reaction flask was then cooled with a surrounding ice bath and acetic acid was added until a pH of 5-6 was obtained. The reaction was then extracted three times with 200 mL portions of methylene chloride, the combined organic layers dried over anhydrous magnesium sulfate, and concentrated to provide crude 6-amino-3-iodophenol (0.533 gms, m.p. 99.5-100.5° C.), which was recrystallized from ethyl ether/hexanes to provide the pure product (0.463 gms, 1.97 mmole, 53% yield; m.p. 126-128° C. (decompose) (reported m.p. 141° C. (Hodgson, H H et al., 1928)); ¹H NMR (CD₃CN, 500 MHz) δ 7.04 (bs, 1H), 6.97 (d, 1H, J=2 Hz), 6.95 (dd, 1H, J=8, 2 Hz), 6.45 (d, 1H, J=8 Hz), 4.05 (bs, 2H); IR (KBr) 3455 (broad), 3380, 3305, 1714, 1504, 1430, 1365, 1279, 1257, 1223, 890 cm⁻¹; Anal. Calcd for C₆H₆INO: C, 30.66; H, 2.57; N, 5.96. Found: C, 30.65; H, 2.42; N, 5.92.).

5′-Iodogeldanoxazinone (17). A mixture of (+)-geldanamycin (4.8 mg, 8.6 μmol) and 2-amino-5-iodophenol (4.0 mg, 0.017 mmol) in glacial acetic acid (1.0 ml) was stirred at 78° C. under nitrogen for 20 hours, then cooled and concentrated. Separation of the deep orange residue by flash chromatography on silica gel (hexane/ethyl acetate) gave a crude product contaminated with unreacted (+)-geldanamycin. This was then dissolved in chloroform and subjected to preparative HPLC separation (Waters Nova-Pak Silica 6 μm 7.8×300 mm column, 2.0 ml/min CHCl₃/EtOAc 2:3) to afford the product as a bright orange powder (2.8 mg, 44%). IR (in CH₂Cl₂) (cm⁻¹) 3139, 3076, 3048, 2995, 2967, 1733, 1684, 1599, 1580, 1496, 1447, 1423, 1260, 1098; ¹H NMR (CDCl₃, 500 MHz, assignment aided by COSY) [symbol] 9.12 (bs, 1H), 8.30 (s, 1H), 7.79 (d, J=2.0 Hz, 1H), 7.71 (dd, J=8.5, 2.0 Hz, 1H), 7.55 (d, J=8.5 Hz, 1H), 7.01 (bd, J=11.5 Hz, 1H), 6.59 (bdd, J=11.5, 11.0 Hz, 1H), 5.94 (bd, J=9.5 Hz, 1H), 5.84 (dd, J=11.0, 10.0 Hz, 1H), 5.20 (s, 1H), 4.71 (bs, 2H), 4.33 (bd, J=10.0 Hz, 1H), 4.24 (bs, 1H), 3.63 (ddd, J=9.0, 6.5, 2.0 Hz, 1H), 3.45 (ddd, J=9.0, 3.0, 3.0 Hz, 1H), 3.36 (s, 3H), 3.26 (s, 3H), 2.79-2.70 (m, 3H), 2.06 (bs, 3H), 1.97-1.84 (m, 2H), 1.82-1.74 (m, 1H), 1.80 (d, J=1.0 Hz, 3H), 0.99 (d, J=7.0 Hz, 3H), 0.97 (d, J=6.5 Hz, 3H); ¹³C NMR (CDCl₃, 125 MHz) [symbol] 180.8, 168.4, 156.0, 148.7, 144.9, 143.3, 136.9, 135.6, 135.3, 135.0, 133.9, 133.6, 133.0, 131.0, 126.8, 126.6, 125.2, 117.5, 112.9, 96.6, 81.9, 81.4, 81.3, 72.2, 57.1, 56.8, 35.4, 33.0, 32.3, 27.7, 23.3, 12.8, 12.6, 12.2; HRMS (FAB) found 746.1937 [M+H]⁺, calcd. 746.1938 for C₃₄H₄₁IN₃O₈.

### EXAMPLE 16

### 11-O-Acetyl-17-(1-azetidinyl)-17-demethoxygeldanamycin (18)

(Schnur et 1., 1995a)). 17-(1-Azetidinyl)-17-demethoxygeldanamycin (3.2 mg, 5.5 mol) was stirred with acetic anhydride (5.2 μl, 0.055 mmol) and DMAP (7.3 mg, 0.060 mmol). Upon the complete conversion of starting material shown by thin layer chromatography (40 hours), the mixture was washed with brine, dried over anhydrous sodium sulfate, and concentrated. Separation by flash column chromatography on silica gel (hexane/ethyl acetate) gave the product as a purple solid (3.2 mg, 93%). IR (in CH₂Cl₂) (cm⁻¹) 3686, 3536, 3420, 3069, 3052, 2930, 1734, 1689, 1649, 1601, 1585, 1549, 1486, 1435, 1374, 1273, 1102; ¹H NMR (CDCl₃, 500 MHz) [symbol] 9.37 (s, 1H), 7.13 (bs, 1H), 6.94 (s, 1H), 6.50 (ddd, J=11.5, 11.0, 1.0 Hz, 1H), 5.81 (dd, J=11.0, 7.5 Hz, 1H), 5.45 (bs, 1H), 5.28 (bd, J=10.0 Hz, 1H), 5.04 (dd, J=8.0, 3.5 Hz, 1H), 4.64-4.54 (m, 4H), 4.48 (bd, J=7.5 Hz, 1H), 3.63 (bs, 1H), 3.33 (s, 3H), 3.31 (s, 3H), 2.85-2.77 (m, 1H), 2.71 (bd, J=10.0 Hz, 1H), 2.38 (quintet, J=8.0 Hz, 2H), 2.06-2.00 (m, 1H), 1.98 (bs, 3H), 1.97 (s, 3H), 1.71-1.56 (m, 2H), 1.68 (bs, 3H), 1.28-1.18 (m, 1H), 0.96-0.93 (m, 6H); ^{[7] 13}C NMR (CDCl₃, 125 MHz) [symbol] 186.2, 178.0, 170.6, 169.2, 155.7, 145.6, 140.4, 135.6, 134.8, 132.9, 128.3, 126.2, 109.2, 108.6, 80.0, 79.2, 78.4, 75.1, 58.5, 57.6, 56.1, 35.8, 33.0, 30.1, 29.7, 21.6, 20.9, 18.5, 15.6, 14.1, 12.3; HRMS (FAB) found 628.3237 [M+H]⁺, calcd. 628.3234 for C₃₃H₄₆IN₃O₉.

### EXAMPLE 17

### 17-(1-Azetidinyl)-7-decarbamyl-17-demethoxygeldanamycin (19)

(Schnur et al., 1994, 1995a, supra). Potassium tert-butoxide (5.3 mg, 0.045 mmol) was added to a solution of 17-(1-azetidinyl)-17-demethoxygeldanamycin (5.0 mg, 8.5 μmol) in tert-butanol (4.0 ml) under nitrogen atmosphere. The reaction was stirred at room temperature for 1 hour, then quenched by partitioning between ethyl acetate and brine. The organic layer was washed with brine, dried over anhydrous sodium sulfate, and concentrated. Separation by flash column chromatography on silica gel (hexane/ethyl acetate) gave the product as a purple solid (4.4 mg, 95%). IR (KBr) (cm⁻¹) 3461, 3330, 2955, 2927, 2871, 2826, 1685, 1652, 1539, 1489, 1404, 1381, 1287, 1255, 1191, 1136, 1106; ¹H NMR (CDCl₃, 500 MHz) [symbol] 9.16 (s, 1H), 7.09 (s, 1H), 6.90 (bd, J=11.5 Hz, 1H), 6.54 (bdd, J=11.5, 11.0 Hz, 1H), 5.98 (dd, J=11.0, 10.0 Hz, 1H), 5.70 (bd, J=9.5 Hz, 1H), 4.72-4.59 (m, 4H), 4.16 (bd, J=10.0 Hz, 1H), 3.98 (s, 1H), 3.52 (dd, J=9.0, 2.0 Hz, 1H), 3.41 (ddd, J=9.0, 3.0, 3.0 Hz, 1H), 3.34 (s, 3H), 3.23 (s, 3H), 2.73 (dqd, J=9.5, 7.0, 2.0 Hz, 1H), 2.57 (d, J=14.0 Hz, 1H), 2.42 (quintet, J=8.0 Hz, 2H), 2.23 (dd, J=14.0, 11.0 Hz, 1H), 2.01 (d, J=1.0 Hz, 3H), 1.77-1.71 (m, 2H), 1.74 (d, J=1.0 Hz, 3H), 1.70-1.62 (m, 1H), 0.97 (d, J=7.0 Hz, 3H), 0.94 (d, J=6.5 Hz, 3H); ¹³C NMR (CDCl₃, 125 MHz) [symbol] 185.8, 178.4, 168.6, 145.8, 140.5, 137.2, 136.1, 134.8, 132.0, 126.9, 125.9, 109.5, 109.2, 81.8, 80.5, 80.3, 72.9, 58.9, 56.7, 56.3, 34.9, 34.2, 32.2, 28.2, 22.9, 18.4, 12.6, 12.4, 11.8; MS (FAB) found 543 [M+H]⁺.

### EXAMPLE 18

### 17,21-Dihydrogeldanamycin (20)

(Schur et al., 1995b). (+)-Geldanamycin (3.5 mg, 6.2 μmol) was dissolved in ethyl acetate (2.5 ml), then aqueous solution (2.5 ml) of sodium dithionite (˜85%, 0.50 g, 2.4 mmol) was added. The mixture was stirred at room temperature. Upon the complete conversion of GA shown by thin layer chromatography (1 hour), the organic layer was separated, washed with brine, dried over anhydrous sodium sulfate, and concentrated. Separation of the solid residue by flash column chromatography on silica gel (hexane/ethyl acetate) afforded a pale yellow solid (3.3 mg, 94%). ¹H NMR (CDCl₃, 500 MHz) δ 8.34 (s, 1H), 8.08 (s, 1H), 8.02 (bs, 1H), 6.76 (bd, J=11.5 Hz, 1H), 6.37 (bdd, J=11.5, 11.0 Hz, 1H), 5.94 (bd, J=9.5 Hz, 1H), 5.64 (dd, J=11.0, 10.0 Hz, 1H), 5.04 (bs, 1H), 4.95 (s, 1H), 4.65 (bs, 2H), 4.29 (bd, J=10.0 Hz, 1H), 3.81 (s, 3H), 3.61 (bd, J=9.0 Hz, 1H), 3.43 (bd, J=9.0 Hz, 1H), 3.33 (s, 3H), 3.21 (s, 3H), 2.79-2.74 (m, 2H), 2.35 (bd, J=14.0 Hz, 1H), 1.82-1.65 (m, 3H), 1.76 (bs, 6H), 0.92 (d, J=6.5 Hz, 3H), 0.86 (d, J=7.0 Hz, 3H); HRMS (FAB) found 562.2886 [M]⁺, calcd. 562.2890 for C₂₉H₄₂N₂O₉.

### EXAMPLE 19

### Halogen-substituted GA Derivatives Prepared from Compound 15: Labeled 17-(2-halo-substituted-ethyl)amino-17-demethoxygeldanamycin Derivatives

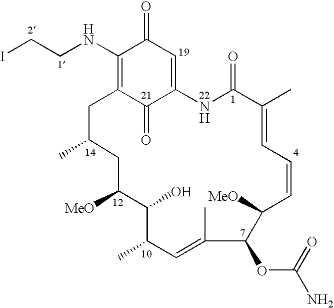

17-(2-Iodoethyl)amino-17-demethoxygeldanamycin. (17-IEG) Phosphoric acid solution (3.0 M, 20.0 μl) was added to a solution of 17-(1-Aziridinyl)-17-demethoxygeldanamycin (17-ARG) (1.1 mg, 1.92 μmol) and potassium iodide (17.4 mg, 0.10 mmol) in dimethylformamide (0.20 ml). After 10 minutes, the mixture was partitioned between ethyl acetate and brine. The organic phase was washed with brine, dried over anhydrous sodium sulfate, and concentrated to give a purple solid (1.3 mg, 97%). IR (KBr) (cm⁻¹) 3466, 3336, 2927, 2824, 1718, 1690, 1652, 1576, 1486, 1374, 1322, 1252, 1188, 1099; ¹H NMR (CDCl₃, 500 MHz) δ 9.09 (s, 1H), 7.30 (s, 1H), 6.94 (d, J=11.5 Hz, 1H), 6.57 (dd, J=11.5, 11.0 Hz, 1H), 6.34 (bt, J=5.0 Hz, 1H), 5.87 (bd, J=9.5 Hz, H), 5.85 (bdd, J=11.0, 10.0 Hz, 1H), 5.18 (s, 1H), 4.73 (br s, 2H), 4.30 (d, J=10.0 Hz, 1H), 4.03 (bs, 1H), 3.91-3.87 (m, 2H), 3.56 (bd, J=9.0 Hz, 1H), 3.44 (ddd, J=9.0, 3.0, 3.0 Hz, 1H), 3.35 (s, 3H), 3.31-3.28 (m, 2H), 3.26 (s, 3H), 2.73 (dqd, J=9.5, 7.0, 2.0 Hz, 1H), 2.69 (d, J=14.0 Hz, 1H), 2.19 (dd, J=14.0, 11.0 Hz, 1H), 2.01 (bs, 3H), 1.80-1.76 (m, 2H), 1.78 (d, J=1.0 Hz, 3H), 1.75-1.69 (m, 1H), 0.99-0.96 (m, 6H); HRMS (FAB) found 700.2099 [M+H]⁺, calcd. 700.2095 for C₃₀H₄₂IN₃O₈.

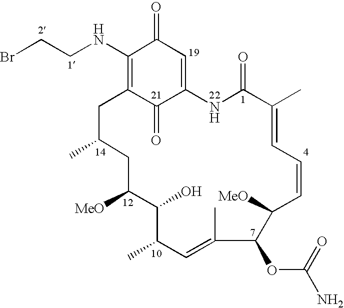

17-(2-Bromoethyl)amino-17-demethoxygeldanamycin. (17-BEG) Phosphoric acid solution (3.0 M, 20.0 μl) was added to a solution of 17-(1-Aziridinyl)-17-demethoxygeldanamycin (17-ARG) (1.1 mg, 1.92 μmol) and potassium bromide (12.8 mg, 0.11 mmol) in dimethylformamide (0.20 ml). After 10 minutes, the mixture was partitioned between ethyl acetate and brine. The organic phase was washed with brine, dried over anhydrous sodium sulfate, and concentrated to give a purple solid (1.2 mg, 96%). IR (KBr) (cm⁻¹) 3460, 3335, 2926, 2850, 2824, 1723, 1691, 1652, 1575, 1487, 1374, 1322, 1254, 1189, 1099; ¹H NMR (CDCl₃, 500 MHz) δ 9.08 (s, 1H), 7.29 (s, 1H), 6.94 (d, J=11.5 Hz, 1H), 6.57 (dd, J=11.5, 11.0 Hz, 1H), 6.36 (bt, J=5.0 Hz, 1H), 5.87 (bd, J=9.5 Hz, H), 5.85 (bdd, J=11.0, 10.0 Hz, 1H), 5.18 (s, 1H), 4.74 (br s, 2H), 4.30 (d, J=10.0 Hz, 1H), 4.03 (bs, 1H), 3.97-3.92 (m, 2H), 3.58-3.52 (m, 3H), 3.44 (ddd, J=9.0, 3.0, 3.0 Hz, 1H), 3.35 (s, 3H), 3.26 (s, 3H), 2.73 (dqd, J=9.5, 7.0, 2.0 Hz, 1H), 2.70 (d, J=14.0 Hz, 1H), 2.23 (dd, J=14.0, 11.0 Hz, 1H), 2.01 (bs, 3H), 1.79-1.76 (m, 2H), 1.78 (d, J=1.0 Hz, 3H), 1.75-1.68 (m, 1H), 0.99-0.96 (m, 6H); HRMS (FAB) found [M+H]⁺, calcd. for C₃₀H₄₂BrN₃O₈.

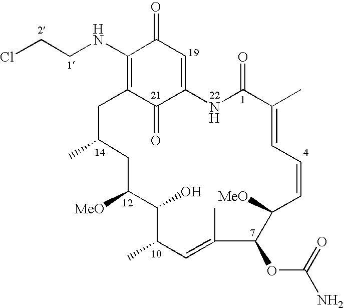

17-(2-Chloroethyl)amino-17-demethoxygeldanamycin. (17-CEG) Hydrochloric acid solution (1.0 M, 20.0 μl) was added to a solution of 17-(1-aziridinyl)-17-demethoxygeldanamycin (17-ARG) (0.1 mg, 0.17 μmol) in dimethylformamide (0.10 ml). After 2 hours, the mixture was partitioned between ethyl acetate and brine. The organic phase was washed with brine, dried over anhydrous sodium sulfate, and concentrated to give a purple solid. TLC of this crude product revealed that the starting material completely converted to the desired title product (major) and 17-HEG (minor).

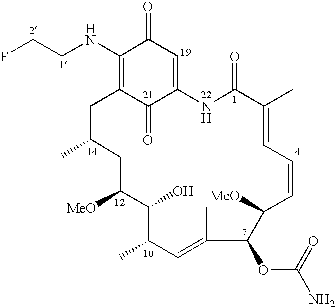

17-(2-Fluoroethyl)amino-17-demethoxygeldanamycin. (17-FEG) Hydrofluoric acid solution (48%, 10.0 μl) was added to a solution of 17-(1-aziridinyl)-17-demethoxygeldanamycin (17-ARG) (0.1 mg, 0.17 μmol) in dimethylformamide (0.10 ml). After 2 hours, the mixture was partitioned between ethyl acetate and brine. The organic phase was washed with brine, dried over anhydrous sodium sulfate, and concentrated to give a purple solid. TLC of this crude product revealed that the starting material completely converted to the desired title product (major) and 17-HEG (minor).

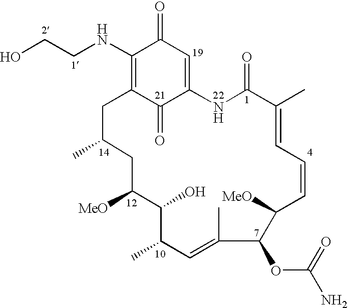

17-(2-Hydroxyethyl)amino-17-demethoxygeldanamycin. (17-HEG) Phosphoric acid solution (3.0 M, 5.0 μl) was added to a solution of 17-(1-aziridinyl)-17-demethoxygeldanamycin (17-ARG) (0.1 mg, 0.17 μmol) in DMSO (0.20 ml) and water (0.05 ml). After 2 hours, the mixture was partitioned between ethyl acetate and brine. The organic phase was washed with brine, dried over anhydrous sodium sulfate, and concentrated to give a purple solid. TLC of this crude product revealed that the starting material completely converted to the desired title product.

### EXAMPLE 20

### Geldanamycin Derivatives and their Inhibitory Activity in the HGF/SF-Met-uPA-Plasmin Cell-Based Assay

Two derivatives of the GA derivative class geldanoxazinone were synthesized and tested for their inhibitory effect (chemical structures shown above). Such derivatives can be prepared by acid-catalyzed condensation of GA with a 2-aminophenol (see Examples above). 5-Bromo-2-aminophenol and 5-iodo-2-aminophenol were used to thus prepare adducts 16 and 17 in 60% and 44% yield, respectively. Each of these latter compounds was found to be inhibitory to the Met signaling pathway only at nanomolar concentrations (<8 IC₅₀). See Table 1.

In an effort to investigate the effect of modification of the ansa ring of GA on activity, an active 17-aminosubstituted-17-demethoxygeldanamycin derivative 17-N-azetidinyl-17-demethoxygeldanamycin (14) was used for making such changes.

TABLE 1
uPA-plasmin inhibition index of compounds.

| | | uPA-plasmin |
|---|---|---|
| | | inhibition |
| Compound | Chemical Name | index* |
| 8 | 17-(2-Fluoroethyl)amino-17- | 19 |
| | demethoxygeldanamycin | |
| 4 | 17-Allylamino-17-demethoxygeldanamycin | 18.0 |
| 15 | 17-N-Aziridinyl-17-demethoxygeldanamycin | 15.7 |
| 6 | 17-Amino-17-demethoxygeldanamycin | 15.3 |
| 14 | 17-N-Azetidinyl-17-demethoxygeldanamycin | 15 |
| 5 | 17-(2-Dimethylaminoethyl)amino-17- | 14.9 |
| | demethoxygeldanamycin | |
| 1 | Geldanamycin | 14.3 |
| 7 | 17-(2-Chloroethyl)amino-17- | 14.0 |
| | demethoxygeldanamycin | |
| 20 | Dihydrogeldanamycin | 12.7 |
| 18 | 11-O-Acetyl-17-N-azetidinyl-17- | 7.9 |
| | demethoxygeldanamycin | |
| 3 | Radicicol | 7.9 |
| 21 | Macbecin II | 6.5 |
| 2 | Macbecin I | 6.4 |
| 13 | 17-Carboxymethylamino-17- | 6.3 |
| | demethoxygeldanamycin | |
| 9 | 17-(2-Acetylaminoethyl)amino-17- | 5.8 |
| | demethoxygeldanamycin | |
| 17 | 5′-Iodogeldanoxazone | 5.8 |
| 12 | 17-(8-Acetamido-3,6-dioxaoctylamino)- | 5.8 |
| | 17-demethoxygeldanamycin | |
| 19 | 17-N-Azetidinyl-7-decarbamyl-17- | 5.7 |
| | demethoxygeldanamycin | |
| 11 | 17-(6-Biotinylaminohexyl)amino-17- | 5.5 |
| | demethoxygeldanamycin | |
| 10 | 17-(6-Acetylaminohexyl)amino-17- | 5.3 |
| | demethoxygeldanamycin | |
| 16 | 5′-Bromogeldanoxazone | 5.3 |

*uPA-plasmin inhibition index or IC₅₀ is the negative log of the drug concentration at which 50% inhibition of uPA occurs when MDCK cells are treated with HGF/SF. Compounds with IC₅₀ higher than 12 are referred to fM-Gai (inhibitors in the fM or lower range) while compounds with index lower than 8 belong to the group known nM-Gai (inhibitors in the nM range).

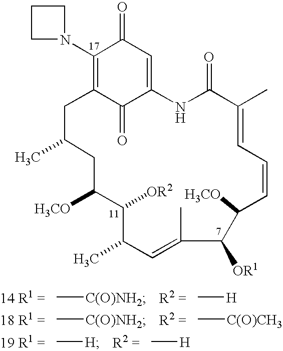 The 11-hydroxyl group of the latter compound could be esterified with acetic anhydride and 4-dimethylaminopyridine to provide 11-O-acetyl-17-N-azetidinyl-17-demethoxygeldanamycin (18).

The 7-urethane group of compound 14 could be removed per slight modification of the Schnur et al. (supra) procedure by treatment with potassium tert-butoxide in tert-butanol (in lieu of the solvent dimethyl sulfoxide, which gave lower product yield) to provide 17-N-azetidinyl-7-decarbamoyl-17-demethoxygeldanamycin (19). Both modifications to the ansa ring led to compounds that exhibited only <8 IC₅₀ Met-uPA-plasmin signaling inhibitory activity (Table 1).

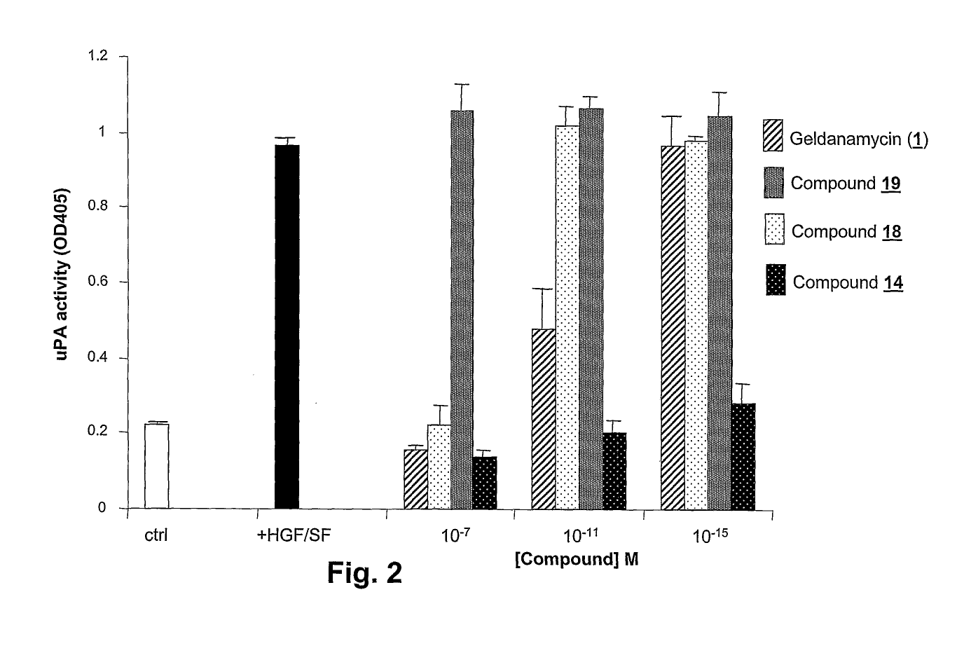

As seen in FIG. 2, compound 14 is highly active (>15 IC₅₀), exceeding the activity of GA, while the modified compound 19 was completely inactive. The activity of compound 18 was <8 IC₅₀.

Finally, studies were done to test the inhibitory activity of the GA-related ansamycin macbecin I (3), and of the hydroquinone forms of the benzoquinone ansamycins, dihydrogeldanamycin (20) and macbecin II (21), as well as of radicicol (3). Results are in Table I. Despite the knowledge that radicicol (Sharma, S V et al., 1998) and macbecins I (Blagosklonny et al., supra)) and II (see herein) have high affinity for hsp90, each of these compounds exhibited poor activity in the present HGF/SF-induced uPA-plasmin assays. However, dihydrogeldanamycin was found to be highly active (>12 IC₅₀).

As mentioned in the Background section, investigations into the therapeutic potential of GA and its derivatives have been focused primarily on biological processes in which hsp90 plays a critical role (Sausville et al, 2003; Workman, 2003; Banerji et al, 2003). Multiple proteins critical to cancer cell survivability and proliferation are dependent on this chaperone protein (Neckers, L et al, 2003; Maloney, A et al, 2003). The ability of GA derivatives to block the function of hsp90 has led to the clinical investigations of 17-N-allylamino-17-demethoxygeldanamycin (4) for cancer treatment (supra). Preliminary reports showed efficacy as an anticancer therapeutic, though hepatic toxicity has reported to be dose-limiting. (Non-dose limiting toxicities included anemia, anorexia, nausea, emesis, and diarrhea.) See, for example, Neckers et al, supra; and Sausville et al, supra.

As disclosed herein, various GA derivatives act as inhibitors of the Met signal transduction pathway in cancer cells at concentrations far below those needed for inhibition of hsp90 function. Additionally, it is disclosed that the inhibitory activity did not always correlate with affinity to human α-hsp90. Although the unknown target(s) of the active GA derivatives disclosed herein remain to be identified, the results suggest certain structure-activity relationships.

Whereas some 17-N-amino-derivatized-17-demethoxygeldanamycin compounds were active in cell-based assays, others were not, notably those with longer 17-N-amino substitutions, e.g., compounds 9, 10, 11, and 12 and the carboxylate derivative 13.

As for ansa ring modifications, when the 7-urethane group was removed from the active GA derivative 14, the resulting decarbamoylated compound 19 was inactive. Crystallographic analysis of GA derivatives 4 and 5 complexed with the N-terminal domain of hsp90 (Stebbins et al, supra; Jez et al, supra) showed that the urethane functionality is undergoes hydrogen bonding interactions with several amino acid residues of hsp90. Additionally, Schnur et al (1995a) reported that the 7-urethane was needed for anti-erbB-2 activity. The 7-urethane of GA derivatives is buried deep in the ATP-binding site of hsp90. Accordingly, the present inventors suggest that the binding site for GA of the unknown target(s) for Met function shares similarities with this binding area of hsp90. Compound 18, made by acetylation of the 11-hydroxyl group of the active GA derivative 14 was inactive in the cell-based assays for Met signaling.

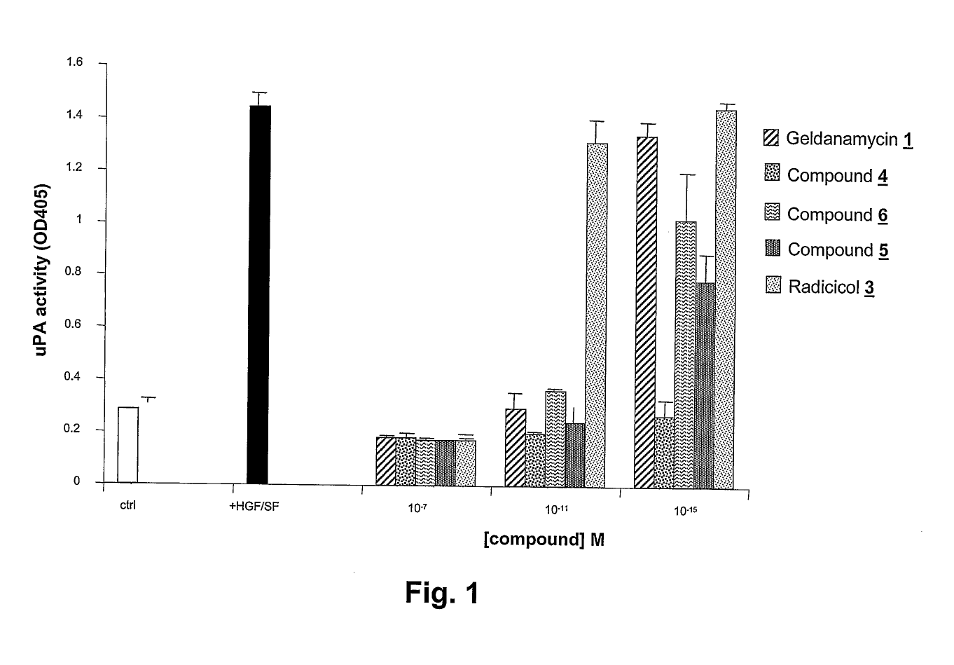

Again, GA is best known for its direct effect on hsp90. The reported cellular effect of GA is such that hsp90 is usually up-regulated and that of Met expression is down-regulated in vitro, as described in Example 21 et seq., below. See also Nimmanapalli, R et al., 2001 and Maulik, G et al., 2002a. This effect of GA's on hsp90 and Met expression levels is disclosed herein only at higher concentrations (<8 IC₅₀). At subnanomolar concentrations (>12 IC₅₀), where uPA activity remains inhibited, there is no change of either hsp90 or Met expression (Examples below). The target of active compounds is different from hsp90, as described below. The cell-based assay used here to detect uPA activity is based upon a HGF/SF induced uPA-plasmin network using MDCK cell lines. Upon treatment with HGF/SF, the uPA activity of MDCK cells is significantly increased (FIGS. 1 and 2; compare Control (“ctl”) vs +HGF/SF). However, this activity is dramatically inhibited by our high activity GA derivatives at femtomolar concentration levels, while radicicol inhibits this activity only at nanomolar levels. (See FIG. 1 for the inhibitory effect of several high activity GA derivatives.)

High activity GA derivatives not only inhibited uPA activity at fM levels, they also inhibited tumor cell invasion in vitro (see Examples below). However, proliferation was only inhibited at mM levels, the same concentrations of the low activity or “nM-GA” derivatives (Webb et al., supra). This suggests that GA's inhibit proliferation and invasion by several mechanisms. For example, proliferation may be affected via inhibition of hsp90 function, whereas invasion is affected by GA interaction with one or more unknown targets.

To support this conception, MDCK cells were intentionally cultured in the presence of macbecin II (21) which inhibits both invasion and proliferation activity at nM levels. MDCK cells were maintained at the highest non-toxic concentrations of macbecin II (21) (3 μM) for several months. Under these conditions, both Met and hsp90 returned to parental (“control”) levels and Met responsiveness to HGF/SF was restored, whereas hsp90 appeared to remain complexed with macbecin. Strikingly, the uPA-plasmin sensitivity to GA's in the macbecin II-treated cells was the same as that in the parental MDCK cells. HGF/SF could still significantly upregulate uPA activity and this could also be inhibited by GA's at fM levels. These findings further confirmed the present inventors' conception that GA inhibits HGF/SF induced uPA activity through non-hsp90 target(s).

The activities observed herein differed from the previously published relative affinities of these compounds with hsp90. For example, the hsp90 high affinity compound radicicol (3) (Roe et al., supra) was inactive in the present cell-based assays whereas the hsp90 binding compounds GA and 17-N-allylamino-17-demethoxyGA (4) were active. Although the target binding site in these cell-based uPA assays remains unknown, the site may also be an ATP-binding site, albeit with some differences.

Kamal et al., supra reported that a high-affinity conformation of hsp90 in tumor cells accounts for the tumor selectivity of 17-N-allylamino-17-demethoxyGA (4) and radicicol (3). The hsp90 of the tumor cells is in multichaperone complexes whereas normal tissue hsp90 is not so complexed. It remains unclear whether the targets at work here are similarly complexed and change the conformation of the GA binding site.

The exquisitely sensitivity of the Met signal transduction pathway to the active compounds, described herein, suggested a catalytic role for the compounds in the disruption of the pathway. Dihydrogeldanamycin (20) was found to be active in the present assays, albeit slightly less so than GA itself. However, compound 20 has been reported to be air-oxidizable to GA (Schnur et al., 1995b, supra), and this cannot be discounted as a possible contributing factor to the activity of compound 20 disclosed herein. However, the related ansamycins macbecin I (2) and its reduction product, macbecin II (21) were both found to be inactive. Both the latter compounds bind hsp90. It is the inventors' view that the active ansamycin derivatives (“fM-GA's”) participate in a catalytic electron-transfer process and that the oxidation-reduction potential between dihydrogeldanamycin (20) and GA is critical for it to be able to do so. The potential difference between the two macbecins may be inadequate for this to occur.

Because of the low concentrations of the highly active GA's that are needed to arrest the Met signaling responsible for the invasive and metastatic behavior of solid tumors, these compounds are attractive drug candidates. The low concentrations at which they are active should eliminate the documented dose-dependent toxicities of GA derivatives. Successful identification and isolation of the targets of such derivatives would allow better screening and design of yet other compounds that would be effective inhibitors of this Met signaling pathway.

### EXAMPLE 21

### Geldanamycins Inhibition of HGF/SF Mediated Tumor Cell Invasion: A. Materials and Methods

Cell Lines and Drugs: MDCK (canine kidney epithelial cells), DBTRG, U373, U118, SW1783 (human glioblastoma cells), SK-LMS-1 (human leiomyosarcoma cells) were purchased from ATCC. DU145, PC-3 (human prostate cancer cells) were from the laboratory of Dr. Han-Mo Koo, Van Andel Research Institute. U87 and SNB19 human glioblastoma cells were from Dr. Jasti Rao, University of Illinois. SNB19 was grown in DMEM F12 medium. All other cells were grown in Dulbecco's Modified Eagle's Medium (DMEM) (both from Gibco®, Invitrogen Corp.). Growth medium was supplemented with 10% fetal bovine serum (FBS; Hyclone) and penicillin and streptomycin.

Geldanamycin and chemical derivatives, 17-(N-allylamino)-17-demethoxygeldanamycin (17-AAG), and 17-amino-17-demethoxygeldanamycin (17-ADG), and Macbecin II (MA) were provided by the National Cancer Institute (NCI) or synthesized as described herein). Radicicol (RA) was purchased from Sigma.

Long term cultures (>3 months) of MDCK cells in growth medium containing MA at 1, 2 and 3×10⁻⁶ M yielded MDCKG1, MDCKG2 and MDCKG3 cells. All compounds were first diluted in DMSO at 0.01M, separated into small stock aliquots (5 μl) and kept at −80° C. until use. When used, stocks were thawed and serially diluted with DMEM/10% FBS. For long term culture with MA, conditioned medium with the compound at 1, 2, or 3×10⁻⁶ M was changed at least twice a week.

HGF/SF-Met-uPA-Plasmin Cell-Based Assay (Webb et al., supra). Cells were seeded in 96-well plates at 1500 cells/well (with the exception of SK-LMS-1 cells, which were seeded at 5000 cells/well) in order to detect color intensity, either with MTS (Promega) for cell growth determination or via Chromozyme PL (Boehringer Mannheim) for uPA-plasmin activity measurement. Cells were grown overnight in DMEM/10% FBS as described previously. Drugs were dissolved in DMSO and serially diluted from stock concentrations into DMEM/10% FBS medium and added to the appropriate wells. Immediately after drug or reagent addition, HGF/SF (60 ng/ml) was added to all wells (with the exception of wells used as controls to calculate basal growth and uPA-plasmin activity levels). Twenty-four hours after drug and HGF/SF addition, plates were processed for the determination of uPA-plasmin activity as follows: Wells were washed twice with DMEM (without phenol red; Life Technologies, Inc.), and 200 μl of reaction buffer [50% (v/v) 0.05 units/ml plasminogen in DMEM (without phenol red), 40% (v/v) 50 mM Tris buffer (pH 8.2), and 10% (v/v) 3 mM Chromozyme PL (Boehringer Mannheim) in 100 mM glycine solution] were added to each well. The plates were then incubated at 37° C., 5% CO2 for 4 h, at which time the absorbances generated were read on an automated spectrophotometric plate reader at a single wavelength of 405 nm. uPA-plasmin inhibition index or IC50 is the negative log₁₀ of the concentration at which uPA-plasmin activity is inhibited by 50%

Proliferation Assay. In parallel with uPA-plasmin detection assay, cell proliferation in 96-well plates was detected with MTS. Cell preparations were the same as described for the uPA-plasmin assay above, except that 15 μl MTS in PMS (phenazine methosulfate) solution (0.92 mg/ml PMS in 0.2 g KCl, 8.0 g NaCl, 0.2 g KH₂PO₄, 1.15 g Na₂HPO₄, 100 mg MgCl₂.6H₂O, 133 mg CaCl₂.2H₂O) was added to each well 24 hours after drug and HGF/SF addition. The plates were then incubated at 37° C., in a 5% CO₂ atmosphere for 4 h. The absorbance was read on an automated spectrophotometric plate reader at 490 nm.

Scatter Assay. In parallel with assessing uPA activity, 96-well plates of MDCK cells were used to detect cell scattering. Cell preparation was same as above (plasmin assay) described above. At the same time as uPA activity was measured, the cells being assayed for scatter were fixed, stained (Diff-Quik Set, Dade Behring AG) and photographed.

In Vitro Cell Invasion Assay. The in vitro invasion assay was performed as previously described by Jeffers et al., 1996, using a 24-well invasion chamber coated with GFR-Matrigel® (Becton Dickinson). Cells were suspended in DMEM/0.1% BSA and were plated in the invasion (upper) chamber (5−25×10³ cells/well) (DBTRG 5,000, SNB19 and U373 25,000 cells/well). The lower chamber was filled with DMEM/0.1% BSA with or without the addition of HGF/SF (100 ng/ml). To evaluate GA inhibition, GA was serially diluted into both the upper and lower chambers at final concentrations 1 μM, to 1 fM as indicated and immediately after HGF/SF addition. After 24 h, cells remaining in the upper chamber were removed by scraping. The cells that invaded through the Matrigel® and attached to the lower surface of the insert were stained using Diff-Quik (Dade Behring Inc.) and counted under a light microscope.

Western Blot and Expression of Met and other Proteins. Cells were seeded in 60×15 mm dishes at 10⁵ cells per dish. HGF/SF (100 ng/ml) was added to each dish 24 hr later. Immediately thereafter, serially diluted GA or MA was added to the relevant dishes at the concentrations indicated, and incubated for the indicated length of time before lysis. For Met and MAPK phosphorylation detection, 10⁵ cells were seeded in 60×15 mm dishes and serum-starved for 24 hrs. After HGF/SF (100 ng/ml) stimulation, cells were lysed at 10 and 30 min. Control cells were not given HGF/SF. After cell lysis, protein concentration was determined by DC protein assay (Bio-Rad), and equal quantities of protein were loaded and separated by SDS-PAGE and transferred in a Western blot to PVDF membranes (Invitrogen). After blocking with 5% dry milk, membranes were blotted with specific antibodies. Antibodies used were: Met (for MDCK cells, Met 25 HZ: purchased from Cell Signaling; for DBTRG, C-28, Santa Cruz Biologicals), phospho-Met (Tyr 1234/1235 rabbit polyclonal antibodies (Cell Signaling), phospho p44/42 MAPK (Thr202/tyr204 rabbit polyclonal antibodies (Cell Signaling), or β-actin (AC-15: ab6276, Abcam) which served as a loading control. After exposure to HRP-conjugated secondary antibody, membranes were incubated with ECL (“Enhanced Chemiluminescence, Amersham Biosciences) and chemiluminescence signal intensity was detected by imaging analysis.

Solid-Phase Binding Assays. GA immobilized affinity gel beads were prepared as follows after Whitesell et al. (1994): GA (1.5 equivalents to affinity gel beads) was stirred with 1,6-diaminohexane (5-10 equivalents) in chloroform at room temperature. Upon the complete conversion of GA (monitored by TLC), the mixture was washed sequentially with dilute aqueous sodium hydroxide and brine. The organic layer was dried over anhydrous sodium sulfate, filtered and concentrated to give 17-(6-aminohexylamine)-17-demethoxygeldanamycin as a dark purple solid (pure by ¹H NMR). The intermediate was then taken up in DMSO and stirred with Affi-Gel 10 beads (Bio-Rad) for two hours. The resulting purple GA-beads were washed with DMSO.

Control beads were made of affinity gel linked with a small chain analogue which does not have affinity for HSP90. Affi-Gel 10 beads (Bio-Rad) were stirred with N-(6-aminohexyl)acetamide (Lee et al., 1995) (1.3 equivalents) in DMSO at room temperature for 2 hours, then washed thoroughly with DMSO.

The above-obtained GA- and control beads were washed in 5 volumes of TNESV (50 mM Tris-HCl (pH 7.5), 20 mM Na₂MoO₄, 0.09% NP-40, 150 mM NaCl, and 1 mM sodium orthovanadate) 3 times and rotated overnight in TNESV at 4° C. to hydrolyze any unreacted N-hydroxysuccinimide, then rocked in 1% BSA in TNESV (1:10) at room temperature for at least 3 hours. After washing thrice more with TNESV, beads were resuspended in 50% TNESV and stored at −78° C.

To perform affinity pull-down experiments, 5×0⁵ cells were seeded in 100×20 mm dishes. After cells grew to 80% confluence, GA or MA, at various concentrations was added to the dishes. After 24 hours, cells were washed twice with PBS and lysed in TNESV buffer supplemented with Complete™ proteinase inhibitors (Roche Molecular Biochemicals). Protein concentration was determined by DC protein assay. Equal quantities of protein were used for Western blotting for Met and HSP90α. For pull-down assays, 20 μl of control or GA beads adjusted for equal concentrations were added to 500 Mμl of extract and rotated at 4° C. overnight. Beads were recovered by low speed centrifugation and washed 3× with TNESV. Sixty μl 2× sample buffer was added to beads and boiled for 10 min. The samples were subjected to SDS-PAGE followed by Western blot analysis.

### EXAMPLE 22

### Geldanamycins are Potent Inhibitors of HGF/SF-Induced uPA Activity in Human Cells

The present inventors' laboratory had previously reported that certain GA derivatives inhibit HGF/SF-induced uPA activity in MDCK cells at very low concentrations (Webb et al., 2000). The most active derivatives, designated “fM-GAi” compounds, are those in which the 17-methoxy group of GA has been replaced by an amino or an alkylamino group (discussed herein).

To determine whether, like MDCK cells, human tumor cells displayed fM-GAi sensitivity, several cell lines were first screened for HGF/SF-inducible uPA activity (Table 2). High levels of uPA activity were induced in MDCK cells by HGF/SF. However, we also identified four human tumor cell lines that exhibited HGF/SF-inducible uPA activity, namely three glioblastoma multiforme (GBM) cell lines (DBTRG, U373 and SNB19) and the highly invasive SK-LMS-1 leiomyosarcoma cells (Jeffers et al., supra; Webb et al., 2000). Detailed fM-GAi concentration-inhibition testing of the compounds listed in Table 1?? were performed using the cell lines shown in Table 2??. Radicicol (RA) and macbecin II (MA) served as examples of drugs that inhibit uPA activation in the nM range. MDCK cells, as previously characterized by Webb et al. supra, were used as a control for fM-GAi drug sensitivity and showed the same sensitivity as previously reported (FIG. 1, panel A) Importantly, only human tumor cell lines that exhibited at least a 1.5-fold level of uPA activation following exposure to HGF/SF (Table 2) were showed similar fM-GAi sensitivity to that of MDCK cells (FIG. 1, panels B (DBTRG), C (U373), and D (SNB19) and data not shown). None of the compounds exhibited significant effects on cell proliferation (FIG. 1, panels E, F, and G). The fM-GAi compounds showed dose-dependency curves extending over a broad concentration range in each cell line, with inhibitory effects for 17-AAG in MDCK and U373 cells observed at concentrations as low as 10⁻¹⁷ M.

These results confirmed that sensitivity to fM-GAi compounds is not a peculiar feature of a particular cell line. However, it also appears that fM-GAi drugs are only effective in cells that attain at least a 50% induction of uPA activity in response to HGF/SF exposure. In the sensitive GBM cell lines, notably DBTRG and U373 cells (FIG. 1, panels B and C, respectively) a reduction in baseline uPA activity was observed in response to fM-GAi compounds. This could be related to low level autocrine HGF/SF-Met signaling found in some GBM cells (Koochekpour et al., 1997).

RA and MA inhibited HGF/SF-mediated induction of uPA activity only at nM or higher concentrations. RA, which displays a much higher binding affinity to HSP90 than does GA (Kd=19 nM vs. 1.2 μM) (Roe et al, 1999; Schulte et al., 1999), inhibited HGF/SF-mediated uPA activity only at nM concentrations. Thus, while HSP90 may be a molecular target for the nM-GAi class of compounds, it cannot account for fM-GAi activity in these sensitive cells.

TABLE 2
HGF/SF Induction of uPA Activity in Selected Cell Lines¹

| | Cell | uPA activity |
|---|---|---|
| Category | lines | induction (fold) |
| Canine kidney epithelial cells | MDCK* | 4.27 |
| Human glioblastoma | DBTRG* | 2.28 |
| | SNB19* | 1.95 |
| | U373* | 1.56 |
| | U118 | 1.12 |
| | U87 | 1.04 |
| | SW1874 | 0.97 |
| Human leiomyosarcoma | SK-LMS-1* | 2.01 |
| Human prostate cancer | DU145 | 1.06 |
| | PC-3 | 1.00 |

¹To measure HGF/SF inducible uPA activity, cells were seeded in 96-well plates. Twenty-four hours later, HGF/SF was added to triplicate wells at final concentrations of 0, 10, 20, 40, and 60 ng/ml and uPA activity was measured after an additional 24 hours of incubation. The values shown are the mean ratios of peak uPA induction observed following HGF/SF exposure to basal uPA activity for each cell line.
Asterisks (*) indicate those cells lines which display fM-GAi sensitivity (FIG. 1, data not shown).

### EXAMPLE 23

### fM-GAi and HGF/SF-Induced Scattering and Invasion

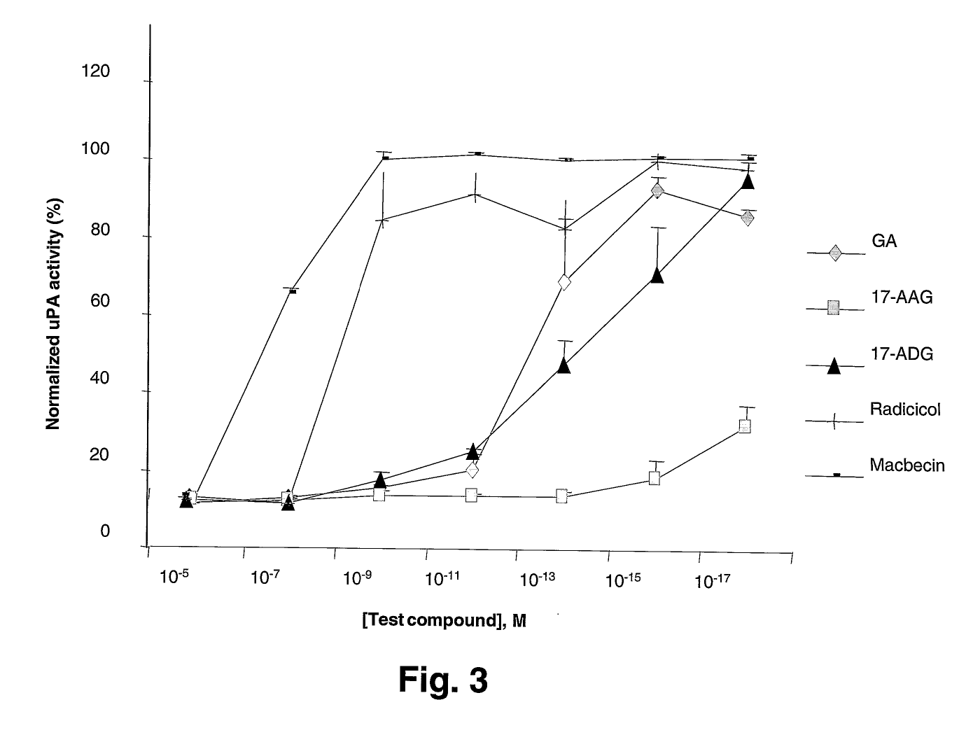

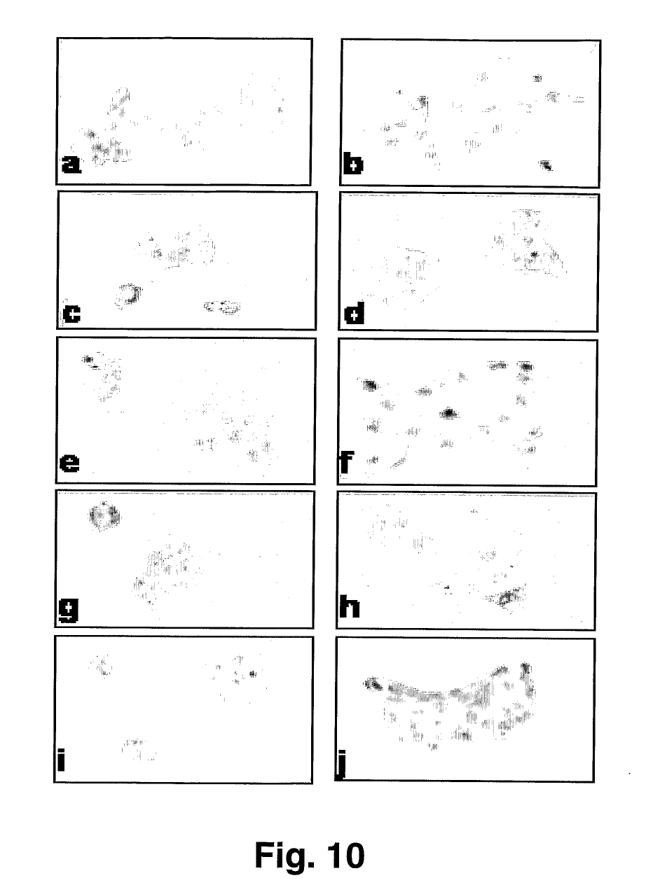

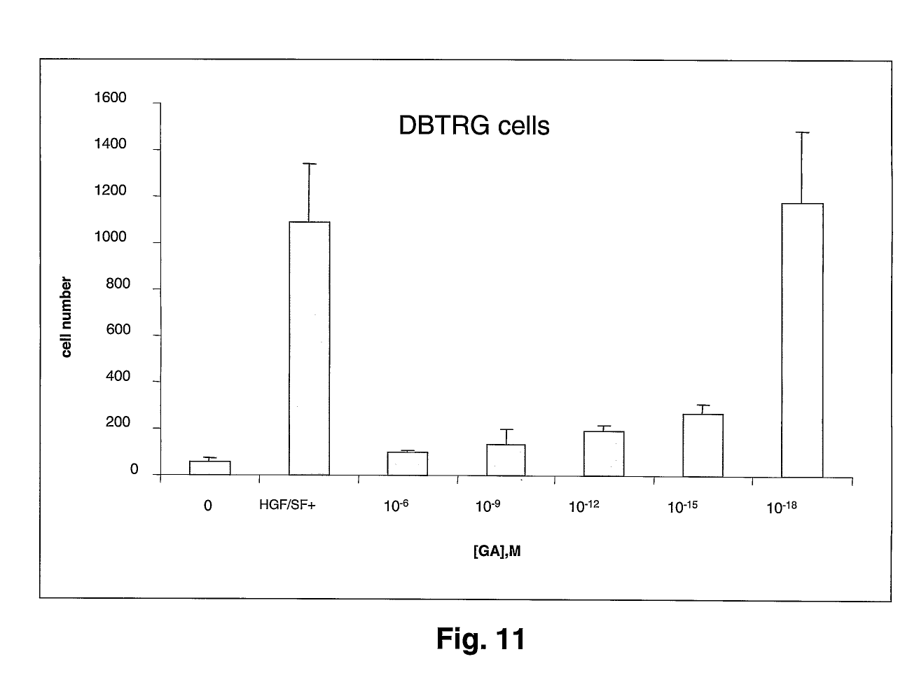

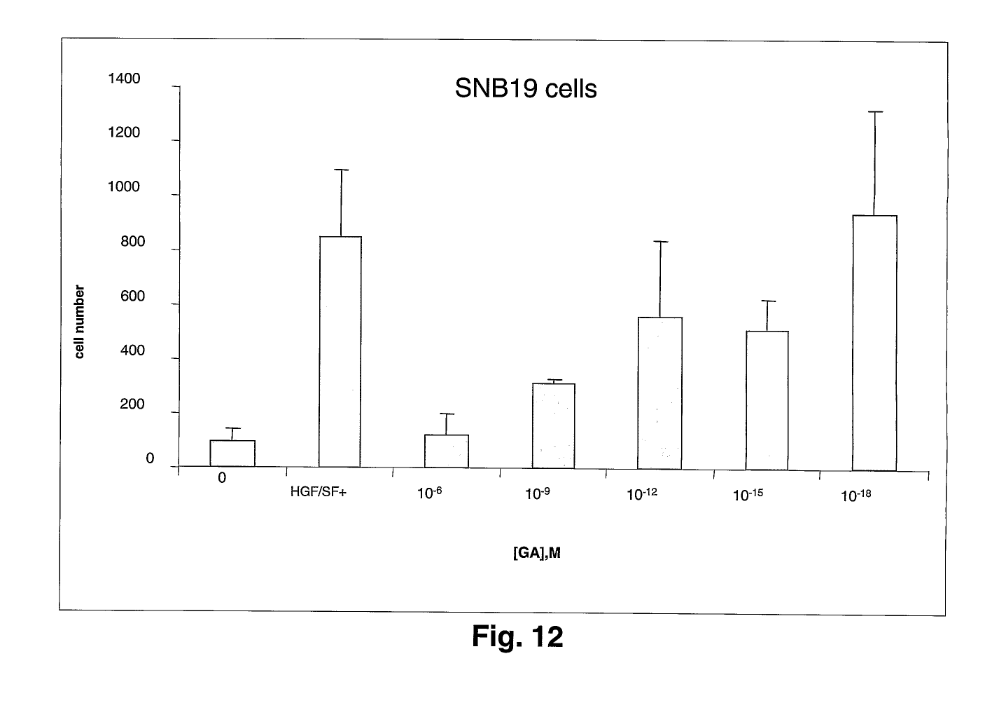

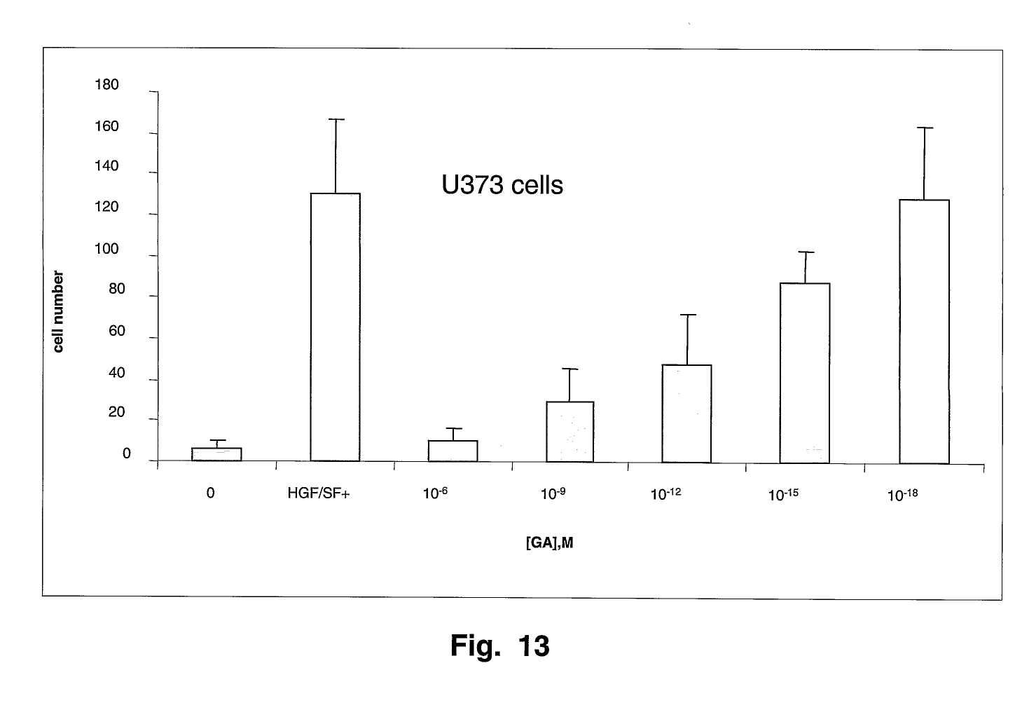

The next study tested whether, in addition to inhibition of uPA activity, fM-GAi compounds affect biological activities of cell scattering and tumor cell invasion in vitro. GA itself and 17-AAG inhibit HGF/SF-induced MDCK cell scattering in the pM to fM range (FIG. 10). Moreover, FIGS. 11-13 show that, even at pM-fM concentrations, GA abolished HGF/SF-induced Matrigel® invasion by the highly invasive DBTRG, SNB19 and U373 human GBM cells. Such marked inhibition of invasion even in the fM range, closely paralleled the inhibitory effects of fM-GAi on HGF/SF induction of uPA activity (cf. FIG. 3-6).

### EXAMPLE 24

### Further Evidence for a Molecular Target Other than HSP90 that Accounts for fM-GAi Activity

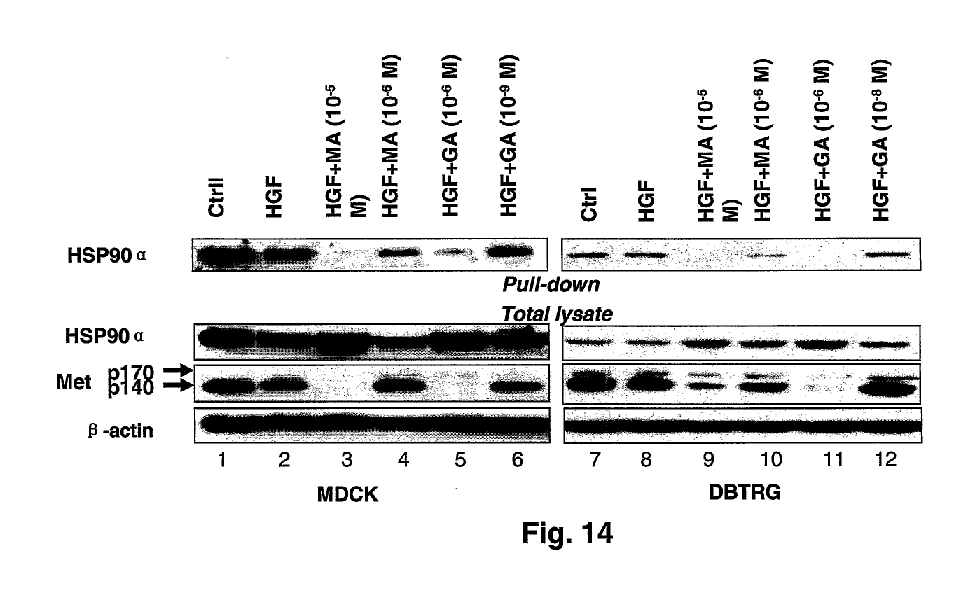

Previous work in the present inventors' laboratory showed that GA inhibited uPAR expression and Met expression in SK-LMS-1 and MDCK cells at nM or higher concentrations (Webb et al., supra), vs the fM range at which inhibition of uPA activity occurred. A study tested the sensitivity of Met and HSP90α expression to GA and MA in cell lines sensitive to fM-GAi compounds (FIG. 14). As reported by others, at nM levels GA up-regulates HSP90α (Nimmanapalli et al., 2001) and downregulates Met expression (Maulik et al., 2002a; Webb et al, supra) (FIG. 3, lanes 5 and 11 for MDCK and DBTRG cells, respectively). However, no significant changes were observed in the relative abundance of either HSP90α or Met at the sub-nM concentrations of fM-GAi compounds like GA (FIG. 14, lanes 6 and 12), at which concentrations uPA activity, scattering or in vitro invasion were inhibited.

GA up-regulation of HSP90α and downregulation of Met were observed at 1-5 M MA (lanes 3 and 9), but less response was observed at 10⁻⁶ M (lanes 4 and 10). Importantly, negligible levels of total HSP90α were recovered with GA-affinity beads at 10⁻⁵ M MA and 10⁻⁶ M GA (lanes 3, 5, 9, and 11), respectively, and the available HSP90α to the GA affinity beads was also reduced with 10⁻⁶ M MA (lanes 4 and 10). These results show that both drugs in the cell lysate effectively competed to prevent association of HSP90 with the bead form of the GA, showing that the available binding sites are blocked. These results led to the conclusion that at sub-nM concentrations of GA, no effect occurs on Met or HSP90α expression. Moreover, the nM-GAi drug MA, like GA, effectively competed with HSP90α binding to GA-affinity beads, even though MA lacks fM-GAi activity. These results indicate that the sub-nM inhibitory effects of fM-GAi compounds cannot involve binding in any stoichiometrically significant way to HSP90α.

### EXAMPLE 25

### Analysis of MDCK Cells Chronically Exposed to Macbecin II

From the preceding experiments showing that HSP90α in MA treated cells was unavailable to GA-affinity beads, it was predicted that if MDCK cultures were maintained chronically on MA at the highest non-toxic level, the binding sites on HSP90α and other nM-GAi target molecules would be occupied, enabling the testing of whether these cells were still sensitive to fM-GAi compounds.

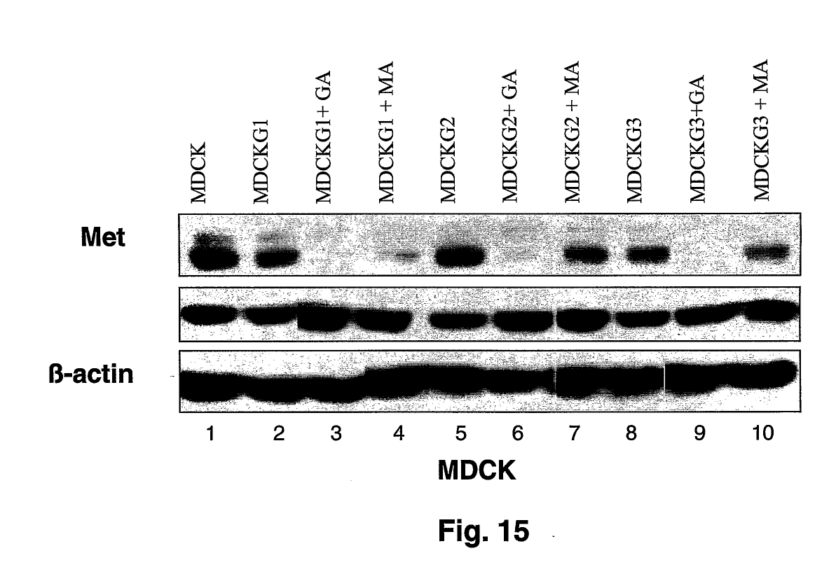

Several high concentrations of MA were tested, but results shown are only those using the highest non-toxic levels that MDCK cells could tolerate and still grow. MDCK cells were cultured long-term in medium containing MA at 1-, 2- and 3×10⁻⁶ M concentrations to generate cells designated MDCKG1, MDCKG2, and MDCKG3, respectively. MDCK cells continued to proliferate at MA concentrations up to, but not above, 3×10⁻⁶ M. All of the cell lines grew well in the presence of MA, albeit at slower rates than parental cells (not shown). In FIG. 15 are displayed the responses of cell lines that had been chronically exposed to MA to an acute challenge with 10⁻⁶M GA or 10⁻⁵ M MA. Cells maintained in 1-2×10⁻⁶ M MA (MDCKG1-G2) exhibited normal levels of both Met and HSP90 (lanes 2, and 5) while Met abundance was lower in MDCKG3 cells (maintained in 3×10⁻⁶M MA) than in parental cells (cf lanes 1 and 8). Upon acute GA challenge for 24 hrs, all of the cell lines chronically exposed to MA showed dramatic decreases in Met abundance, with less of a decrease evident upon challenge with 10⁻⁵ M MA itself, especially with MDCKG2 and -G3 cells (lanes 3, 6, and 9). Acute increases in HSP90α were suggested in MDCKG1 AND -G2 cell lines upon GA challenge, but not in MDCKG3 cells. From these results it is concluded that the MDCKG3 were rendered at least partially tolerant to 10⁻⁶ M GA while MDCKG1 and G2 as are less so and, in large measure, are more like the parental cells (cf. FIGS. 14 and 15).

### EXAMPLE 26

### Met Function in Cells Chronically Exposed to Macbecin II

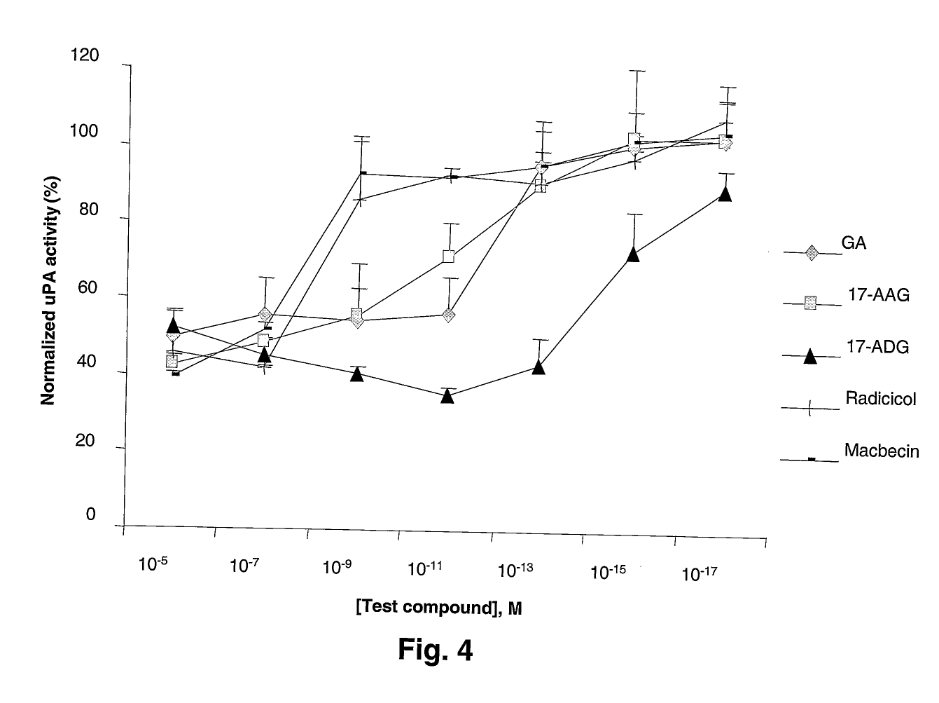

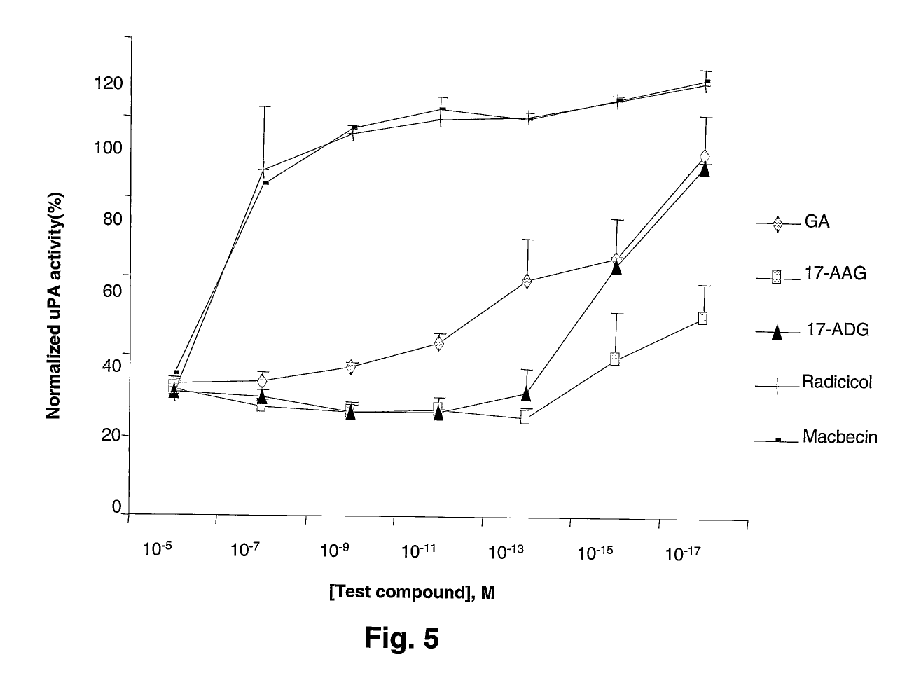

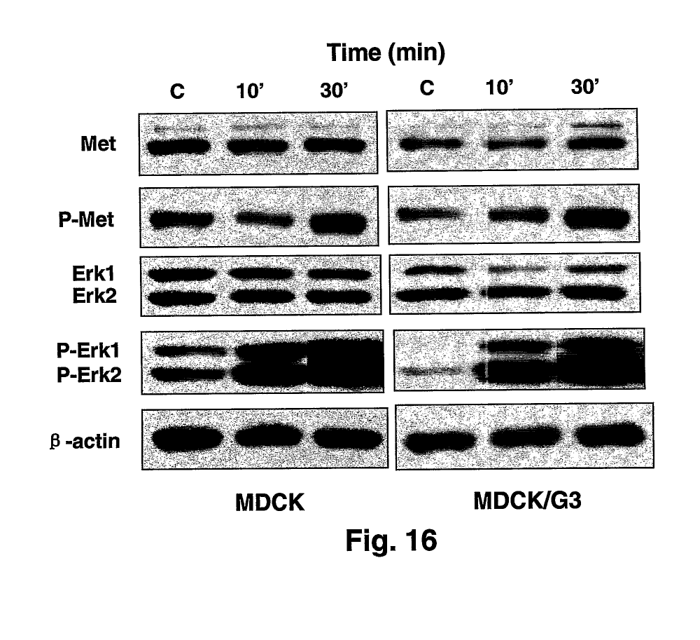

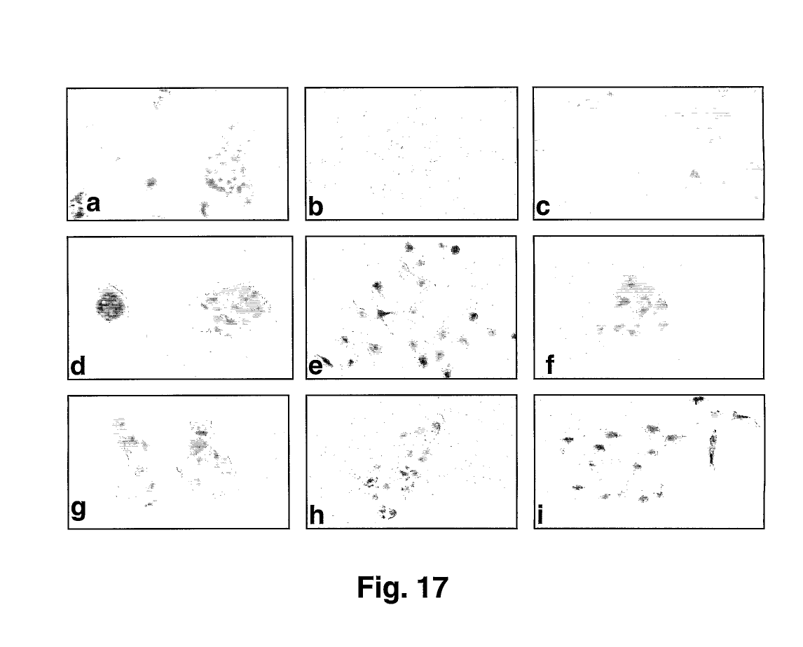

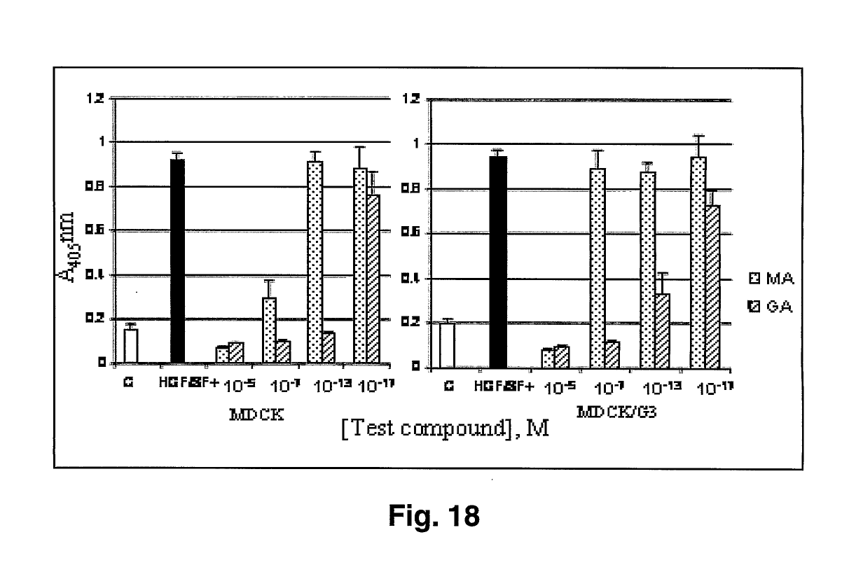

To assess whether MA-treated MDCKG3 cells retained their sensitivity to GA, a study was done which first tested whether Met remained functional in cells chronically exposed to MA. Met function was measured as HGF/SF-induced downstream signaling (FIG. 16), scattering activity (FIG. 17), and induction of uPA activity (FIG. 18). Parental MDCK cells and MDCKG3 cells showed comparable time courses of Erk1 and Erk 2 phosphorylation after HGF/SF stimulation (FIG. 16) as well as similar levels and time courses of Met phosphorylation. Thus, despite slightly lower levels of Met expression in MDCKG3 cells (FIGS. 4 and 5), HGF/SF-induced Met and Erk1 and Erk2 phosphorylation patterns are comparable to those of MDCK parental cells.

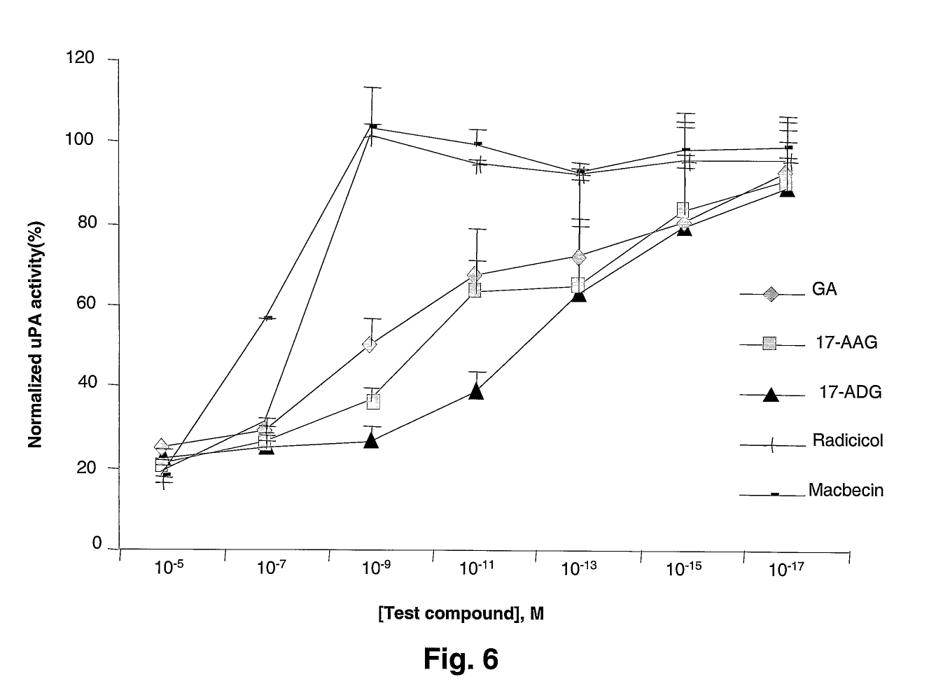

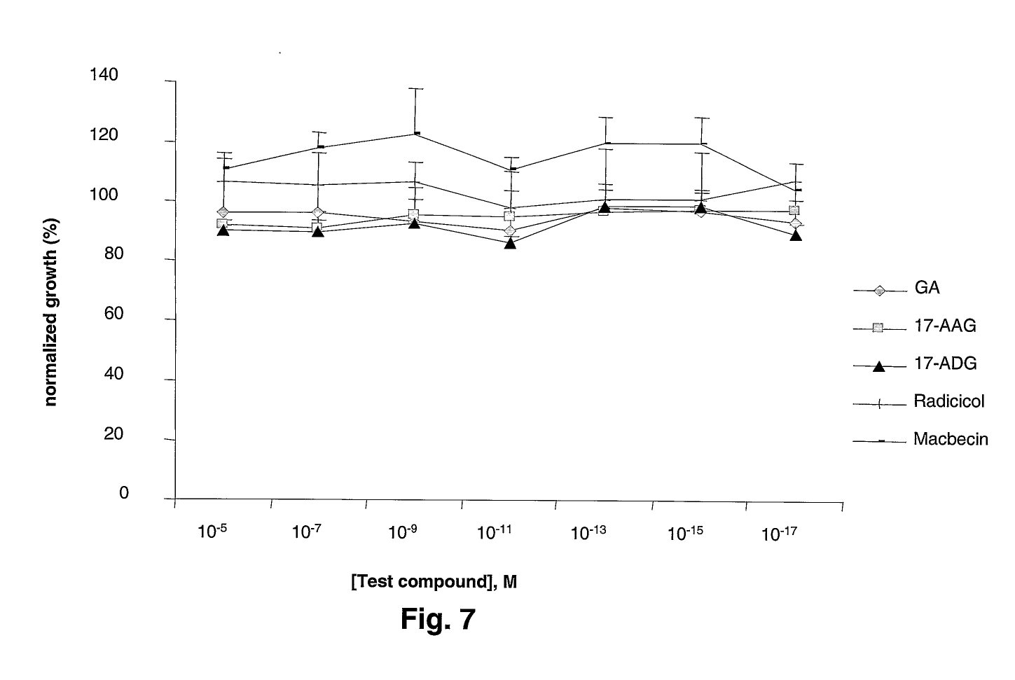

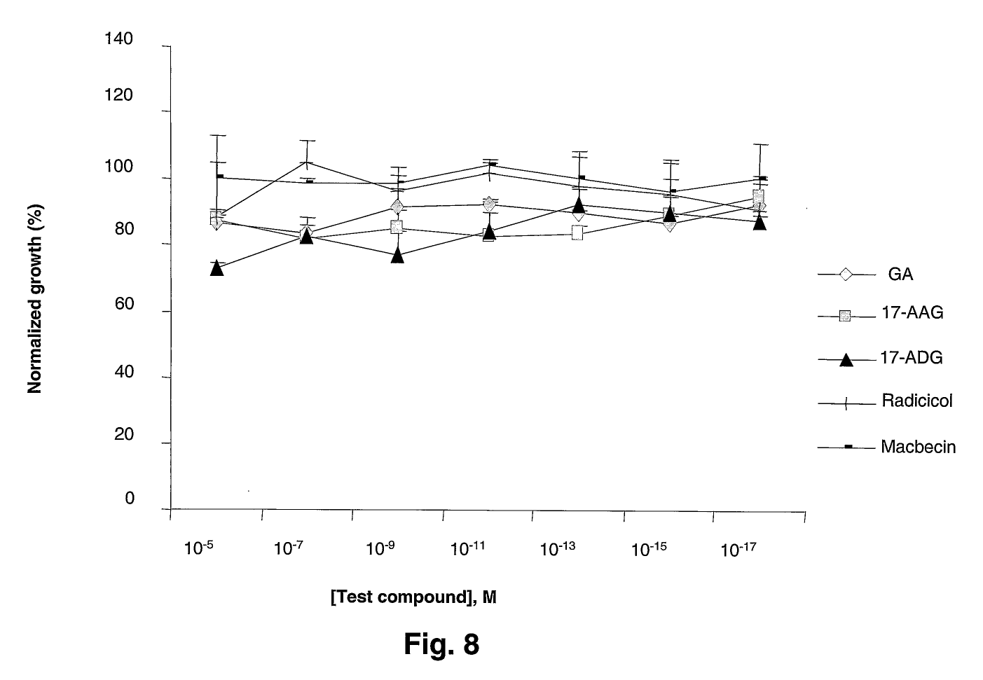

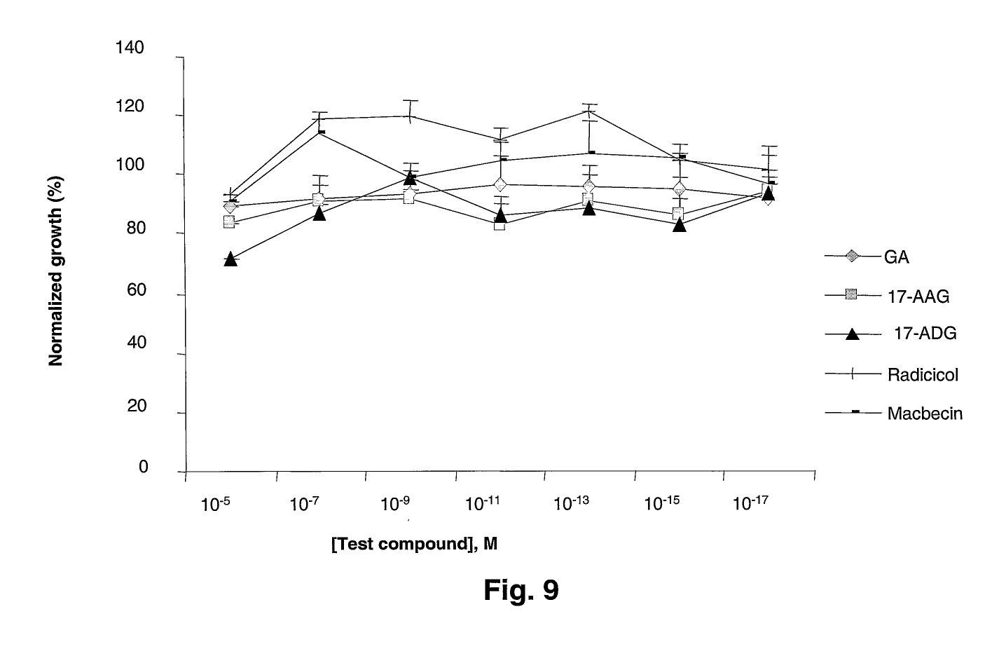

MDCKG3 cells still scattered in response to HGF/SF even in the presence of 3×10⁻⁶ M MA (FIG. 6A, panels d and e), while the same concentration of MA effectively blocked scattering of MDCK cells (FIG. 17, panel c).

GA inhibitory activity at 10⁻⁷ to 10⁻¹⁵ M on HGF/SF-induced cell scattering in MDCKG3 cells was tested next (FIG. 17). Only at 10⁻¹⁵ M GA, was scattering again fully observed (FIG. 6A, panel i), showing that exquisite sensitivity to fM-GAi persists even in MDCKG3 cells maintained in 3×10⁻⁶ MA.

The next experiment tested whether Met remained functional in MDCKG3 cells chronically exposed to MA, as measured by HGF/SP-induced downstream uPA induction (FIG. 18). Just as in parental MDCK cells, GA was a far more potent inhibitor of HGF/SF-induced uPA activity than was MA; it was effective at 10⁻³ M in MDCKG3 cells. Taken together, these findings show that Met in MDCKG3 cells is fully responsive to HGF/SF in signaling through Erk1 and Erk 2, both by scattering activity and uPA induction.

### DISCUSSION OF EXAMPLES 22-26

HGF/SF-induced uPA activity is known to be correlated with tumor invasion and metastasis in many types of solid tumors. When Met signaling is initiated by HGF/SF, both uPA and uPAR expression are up-regulated and plasminogen is cleaved into plasmin, leading to degradation of the extracellular matrix (Ellis et al., 1993). High-levels of uPA and uPAR expression are associated with poor clinical prognosis (Duffy, 1996; Duffy et al., 1996; Harbeck et al., 2002), and, indeed, uPAR-targeted anti-cancer strategies are being developed (Gondi et al., 2003; Lakka et al., 2003; Schweinitz et al., 2004). The present inventors and colleagues previously developed a cell-based method for screening HGF/SF-induced uPA-plasmin network inhibitors and, using this assay, discovered that fM-GAi compounds can inhibit HGF/SF-induced uPA-plasmin proteolysis at fM concentrations in MDCK cells (Webb et. al., supra).

The Examples above show that not only uPA-plasmin activity, but also HGF/SF induced scattering was inhibited by fM-GAi at fM levels (FIG. 2 A). MDCK cells appeared to be the most sensitive indicator of these highly potent effects, both in HGF/SF-induced scattering assays and uPA-plasmin induction (Table 2).

The present inventors found that, with a mouse mammary cancer cell line DA3 and a human prostate cancer cell line DU145, both cell lines scattered in response to HGF/SF but uPA activity was not induced by HGF/SF and the scattering was only inhibited at nM. The explanation for this result is that HGF/SF inducible scattering and uPA-plasmin up-regulation are linked to the fM-GAi sensitivity as indicated from the results in Table 2 and FIG. 1. MDCK cells remain a better test system for detecting fM-GAi effects on scattering.

Also disclosed herein for the first time was the fM-GAi-mediated uPA inhibition in four human tumor cell lines that respond to HGF/SF. Hence, these potent effects are a property of human tumor cells as well, not something peculiar to MDCK cells. In the sensitive human cell lines, uPA activity was upregulated by HGF/SF by at least 1.5 fold, a level that appears to be necessary for reliably measuring fM-GAi inhibition. In fM-GAi sensitive glioblastoma (GBM) cell lines, there occurred a marked reduction in baseline uPA activity, a reduction that does not occur in insensitive cell lines even though the baseline uPA activity may be higher than in the sensitive cell lines. Many GBM cell lines express HGF/SF and Met in an autocrine manner (Koochekpour et al., supra), whereas none of the “insensitive” cells do so. Thus, the reduction in baseline may be explained by the exquisitely potent activity of the fM-GAi drugs being directed at an HGF/SF induced pathway. In addition, the fM-GAi compounds inhibit invasion (in vitro) in all 3 sensitive GBM cells in parallel with uPA inhibition, confirming the causal relatedness of uPA inhibition and tumor invasion and metastasis.

At nM concentrations, members of the GA drug family inhibit tumor growth by interfering with HSP90α chaperone function leading to degradation of improperly folded oncoproteins (Chavany et al., 1996; Stebbins et al., 1997; Whitesell & Cook, 1996). Most of the identified cellular oncoproteins bind to HSP90 via the amino-terminal ATP binding domain, which is also the GA binding domain (Chavany et al., supra; Mimnaugh et al., 1996; Schneider et al., 1996; Schulte et al., 1997). Typically, in cells treated with nM concentrations of GA, HSP90 expression is up-regulated and oncoproteins are degraded within 24 hours. GA treatment induces oncoprotein degradation within 6 to 24 hours (Liu et al., 1996; Maulik et al., supra; Nimmanapalli et al., 2001; Tikhomirov & Carpenter, 2000; Yang et al., 2001), accompanied by up-regulation of HSP90α expression (Nimmanapalli et al., 2001). Yet in a human small cell lung cancer (SCLC) cell line, GA treatment resulted in Met degradation even when HSP90 expression did not change (Maulik et al., supra).

In contrast, it is shown here that scattering, invasion and uPA activity are inhibited by fM-GAi compounds at concentrations that are much too low to cause either HSP90 upregulation or Met downregulation. Also, fM-GAi compounds inhibit uPA activity even when added up to 4 hrs after HGF/SF addition, even though phosphorylation of key signaling components occurs as early as 10 min after HGF/SF addition. Therefore, it has been shown herein that fM-GAi inhibition must occur downstream to Met signaling.

RA, with a higher HSP90 binding affinity than GA, only shows nM-GAi uPA inhibition. RA binds to the same ATP pocket of HSP90 as does GA and the fM-GAi compounds, but with higher affinity (Roe et al., 1999; Schulte et al., 1999). This finding suggests that fM-GAi compounds inhibit HGF/SF-induced uPA activity, cell scattering, and tumor cell invasion through non-HSP90 targets. The concurrent inhibition of these three activities suggests that fM-GAi drugs target a common step in the HGF/SF-regulated migration/invasion pathway. It is not inconceivable that a rare subset of HSP90 chaperones is responsible for the fM-GAi inhibition. For example, Eustace et al. (2004) reported that an HSP90α isoform has an essential role in cancer invasiveness, and that this isoform is expressed extracellularly and interacts outside the cell to promote MMP2 activation.

To test whether this form of HSP90α was possibly also responsible for the sensitive uPA effects described here, a study was done using GA-beads in the uPA assay. Inhibition of HGF/SF-induced uPA activity with extracellular GA affinity beads only occurred at 10⁻⁵ M, provig that fM level inhibition of uPA is not related to such an HSP90α extracellular isoform. According to this invention, there is a novel molecular target for fM-GAi drugs.

Glioblastomas are highly invasive tumors, and HGF/SF stimulation of the uPA-plasmin network is a key step in GBM invasion (Gondi et al., 2003; Rao, 2003). These tumors infiltrate normal brain tissue and propagate along blood vessels, such that it is impossible to completely resect them. Eighty percent of GBM tumors express HGF/SF, while 100% overexpress Met (Birchmeier et al., supra). uPA activity was found to be higher in astrocytomas (particularly in glioblastomas) than in normal brain tissue or in low-grade gliomas. (Bhattacharya et al., 2001; Gladson et al., 1995; Yamamoto et al., 1994), and elevated uPA expression is a poor prognostic indicator (Zhang et al., 2000). Therefore, drugs that target Met and uPA may be important for new therapeutic strategies (Rao, 2003). Previous study of some of the present inventors and colleagues measured the invasive potential in several GBM cell lines; DBTRG and U373 were the most invasive lines (Koochekpour et al, 1997). SNB19 cells are also a highly invasive GBM cell line (Lakka et al., 2003). As shown here, all three invasive GBM cell lines showed fM-GAi inhibition of HGF/SF-induced uPA activity and invasion at extremely low concentrations. 17-AAG is currently in clinical trials for several different cancers (Blagosklonny, 2002; Goetz et al., 2003) but not glioblastoma. According to the present invention, fM-GAi drugs are useful for the treatment of GBM brain cancer.

### REFERENCES CITED ABOVE

### As Abbreviated Citations

Abounader, R, Ranganathan, S, Lal, B, Fielding, K, Book, A, Dietz, H, Burger, P, Laterra, J. (1999) J Natl Cancer Inst 91:1548-1556
Abounader, R, Lal, B, Luddy, C, Koe, G, Davidson, B, Rosen, E. M, Laterra, J. (2002) FASEB J 16:108-110
Allen, C F, Spangler, F W, Webster, E R. (1963) Org Synth Coll 4:433
Banerji, U, Judson, I, Workman, P. (2003) Curr Cancer Drug Targets 3:385-390
Bhattacharya, A, Lakka, S S, Mohanam, S, Boyd, D, Rao, J S (2001) Clin Canc Res 7:267-76
Birchmeier, C, Birchmeier, W, Gherardi, E, Vande Woude, G F (2003) Nat Rev Mol Cell Biol 4:915-25
Blagosklonny, M V, Toretsky, J, Bohen, S, Neckers, L. Proc Natl Acad Sci USA 1996 93:8379-8383
Blagosklonny, M V (2002) Leukemia 16:455-62
Bohen, S P (1998), Mol Cell Biol 18:3330-3339
Bonvini, P, An, W G, Rosolen, A, Nguyen, P, Trepel, J, Garcia de Herreros, A, Dunach, M, Neckers, L M (2001) Canc Res 61:1671-7
Boyland, E, Sims, P. (1954) J Chem Soc 980-985
Cao, B, Su, Y, Oskarsson, M, Zhao, P, Kort, E J, Fisher, R. J, Wang, L M, Vande Woude, G F (2001) Proc Natl Acad Sci USA 98:7443-7448
Chavany, C, Mimnaugh, E, Miller, P, Bitton, R, Nguyen, P, Trepel, J, Whitesell, L, Schnur, R, Moyer, J, Neckers, L (1996) J Biol Chem 271:4974-7
Christensen, J. G, Schreck, R, Burrows, J, Kuruganti, P, Chan, E, Le, P, Chen, J, Wang, X, Ruslim, L, Blake, R, Lipson, K. E, Ramphal, J, Do, S, Cui, J. J, Chemington, J. M, Mendel, D. B. (2003) Canc Res 63:7345-7355
Danilkovitch-Miagkova, A, Zbar, B. (2002) J Clin Invest 109:863-867
Date, K, Matsumoto, K, Shimura, H, Tanaka, M, Nakamura, T. (1997) FEBS Lett 420:1-6
DeBoer C, Meulman P A, Wnuk R J, Peterson D H. (1970), J Antibiot (Tokyo) 23:442-7
Duffy, M J (1996) Clin Canc Res 2:613-618
Duffy, M J, Duggan, C, Maguire, T, Mulcahy, K, Elvin, P, McDermott, E, Fennelly, J J O'Higgins, N (1996) Enzyme Protein 49:85-93
Dutta, R, Inouye, M. (2000) Trends Biochem Sci 25:24-28
Egorin M J, Rosen D M, Wolff J H, Callery P S, Musser S M, Eiseman J L (1998) Canc Res 58:2385-2396
Egorin, M J, Lagattuta, T F, Hamburger, D R, Covey, J M, White, K D, Musser, S M, Eiseman, J L. (2002) Cancer Chemother Pharmacol 49:7-19
Ellis, V, Behrendt, N, Dano, K (1993) Methods Enzymol 223:223-33
Eustace, B K, Sakurai, T, Stewart, J K, Yimlamai, D, Unger, C, Zehetmeier, C, Lain, B, Torella, C, Henning, S W, Beste, G, Scroggins, B T, Neckers, L, Bag, L L, Jay, D G (2004) Nat Cell Biol 6:507-14
Fink, A L. (1999) Physiol Rev 79 425-449
Gladson, C L, Pijuan-Thompson, V, Olman, M A, Gillespie, G Y & Yacoub, I Z (1995) Am J Pathol 146:1150-60
Goetz, M P, Toft, D O, Ames, M M, Erlichman, C (2003) Ann Oncol 14:1169-76
Gondi, C S, Lakka, S S, Yanamandra, N, Siddique, K, Dinh, D H, Olivero, W C, Gujrati, M, Rao, J S (2003) Oncogene 22:5967-75
Grenert, J P, Sullivan, W P, Fadden, P, Haystead, T A, Clark, J, Minmaugh, E, Krutzsch, H, Ochel, H J, Schulte, T W, Sausville, E, Neckers, L M, Toft, D O. (1997) J Biol Chem 272:23843-23850
Hanzlik, R P, Weller, P E, Desai, J, Zheng, J, Hall, L R, Slaughter, D E. (1990) J Org Chem 55:2736-2742
Harbeck, N, Kates, R E, Look, M P, Meijer-Van Gelder, M E, Klijn, J G, Kruger, A, Kiechle, M, Janicke, F, Schmitt, M, Foekens, J A (2002) Canc Res 62:4617-22
Hattori, N, Mizuno, S, Yoshida, Y, Chin, K, Mishima, M, Sisson, T H, Simon, R H, Nalkmura, T, Miyake, M (2004) Am J Pathol 164:1091-8
Hodgson, H H, Moore, F H. (1926) J Chem Soc 155-161
Hodgson, H H, Moore, F H. (1927) J Chem Soc 630-635
Hodgson, H H, Kershaw, A. (1928) J Chem Soc 2703-2705
Ivy, P S, Schoenfeldt, M. (2004) Oncology (Huntingt) 18:610 615 619-20
Jeffers, M, Rong, S, Vande Woude, G F (1996) Mol Cell Biol 16:1115-25
Jez, J M, Chen, J C, Rastelli, G, Stroud, R M, Santi, D V. (2003) Chem Biol 10:361-368
Jiang, W. G, Grimshaw, D, Lane, J, Martin, T. A, Abounder, R, Laterra, J, Mansel, R. E. (2001) Clin Canc Res: 7:2555-2562
Kamal, A, Thao, L, Sensintaffar, J, Zhang, L, Boehm, M F, Fritz, L C, Burrows, F J. (2003) Nature 425:407-410
Koochekpour, S, Jeffers, M, Rulong, S, Taylor, G, Klineberg, E, Hudson, E A, Resau, J H, Vande Woude, G F (1997) Canc Res 57:5391-8
Lakka, S S, Gondi, C S, Yanamandra, N, Dinh, D H, Olivero, W C, Gujrati, M, Rao, J S (2003) Canc Res 63:2454-61
Lee, Y B, Park, M H, Folk, J E (1995) J Med Chem 38:3053-61
Li, L H, Clark, T D, Cowie, C H, Rinehart, K L. (1977) Cancer Treat Rep 61:815-824
Liu, Z G, Hsu, H, Goeddel, D V, Karin, M (1996) Cell 87:565-76
Maloney, A, Workman, P. (2002) Expert Opin Biol Ther 2:3-24
Maloney, A, Clarke, P A, Workman, P. (2003) Curr Cancer Drug Targets 3:331-341
Matsumoto, K, Nakamura, T. (2003) Cancer Sci: 94:321-327
Maulik, G, Kijima, T, Ma, P C, Ghosh, S K, Lin, J, Shapiro, G I, Schaefer, E, Tibaldi, E, Johnson, B E, Salgia, R (2002a). Clin Canc Res 8:620-6277
Maulik, G, Shrikhande, A, Kijima, T, Ma, P C, Morrison, P T, Salgia, R (2002b) Cytokine Growth Factor Rev 13:41-59
Mimnaugh, E G, Chavany, C, Neckers, L (1996) J Biol Chem 271:22796-801
Morotti, A, Mila, S, Accornero, P, Tagliabue, E, Ponzetto, C. (2002) Oncogene: 21:4885-4893
Munster P N, Basso A, Solit D, Norton L, Rosen N (2001) Clin Canc Res 7:2155-2158
Muroi, M, Izawa, M, Kosai, Y, Asai, M. (1980) J Antibiot 33:205-212
Neckers, L, Ivy, S P. (2003) Curr Opin Oncol 15:419-24
Nimmanapalli, R, O'Bryan, E, Bhalla, K. (2001) Canc Res 61:1799-1804
Ochel, H J, Eichhorn, K, Gademann, G (2001) Cell Stress Chaperones 6:105-12
Picard, D, (2002) Cell Mol Life Sci 59:1640-1648
Pratt, W B, Toft, D O, (2003) Exp Biol Med 228:111-133
Rao, J S (2003) Nat Rev Cancer 3:489-501
Richter, K, Buchner, J. (2001) J Cell Physiol 188:281-290
Rinehart, K L., Jr, W, M M, Witty, T R, Tipton, C D, Shield, L S. (1977) Bioorg Chem 6:353-369
Roe, S M, Prodromou, C, O'Brien, R, Ladbury, J E, Piper, P W, Pearl, L H, (1999) J Med Chem 42:260-266
Rong, S, Segal, S, Anver, M, Resau, J. H, Woude, G. F. V. (1994) Proc Natl Acad Sci USA 91:4731-4735
K Sasaki K, Rinehart K L, Slomp G, Grostic M F, Olson E C, (1970) J Am Chem Soc 92:7591-7593 (1970))
Sasaki, K, Yasuda, H, Onodera, K. (1979) J Antibiot 32:849-851
Sausville, E A, Tomaszewski, J E, Ivy, P. (2003) Curr Cancer Drug Targets 3:377-383
Schneider, C, Sepp-Lorenzino, L, Nimmesgern, E, Ouerfelli, O, Danishefsky, S, Rosen, N, Hartl, F U (1996) Proc Natl Acad Sci USA 93:14536-41
Schnur, R C, Corman, M L. (1994) J Org Chem 59:2581-2584
Schnur, R C, Corman, M L, Gallaschun, R J, Cooper, B A, Dee, M F, Doty, J L, Muzzi, M L, DiOrio, C I, Barbacci, E G, Miller, P E, Pollack, V A, Savage, D M, Sloan, D E, Pustilnik, L R, Moyer, J D, Moyer, M P. (1995a) J Med Chem 38:3813-3820
Schnur, R C, Corman, M L, Gallaschun, R S, Cooper, B A, Dee, M F, Doty, J L, Muzzi, M L, Moyer, J D, DiOrio, C I, Barbacci, E G, Miller, P E, O'Brien, A T, Morin, M J, Foster, B A, Pollack, V A, Savage, D M, Sloan, D E, Pustilnik, L R, Moyer, M P. (1995b) J Med Chem 38:3806-3812
Schulte, T W, An, W G, Neckers, L M (1997) Biochem Biophys Res Commun 239:655-9
Schulte, T W, Akinaga, S, Soga, S, Sullivan, W, Stensgard, B, Toft, D, Neckers, L M. (1998) Cell Stress Chaperones 3:100-108
Schulte, T W, Akinaga, S, Murakata, T, Agatsuma, T, Sugimoto, S, Nakano, H, Lee, Y S, Simen, B B, Argon, Y, Felts, S, Toft, D O, Neckers, L M, Sharma, S V (1999) Mol Endocrinol 13:1435-48
Schulte, T W, Neckers, L M, (1998) Cancer Chemother Pharmacol 42:273-279
Schweinitz, A, Steinmetzer, T, Banke, I J, Arlt, M, Sturzebecher, A, Schuster, O, Geissler, A, Giersiefen, H, Zeslawska, E, Jacob, U, Kruger, A, Sturzebecher, J (2004) J Biol Chem
Sharma, S V, Agatsuma, T, Nakano, H. (1998) Oncogene 16:2639-2645
Solit, D B, Zheng, F F, Drobnjak, M, Munster, P N, Higgins, B, Verbel, D, Heller, G, Tong, W, Cordon-Cardo, C, Agus, D B, Scher, H I, Rosen, N (2002) Clin Cane Res 8:986-93
Stabile, L. P, Lyker, J. S, Huang, L, Siegfried, J. M. (2004) Gene Ther: 11:325-335
Stebbins, C E, Russo, A A, Schneider, C, Rosen, N, Hartl, F U, Pavletich, N P. (1997) Cell: 89:239-50
Tacchini, L, Matteucci, E, De Ponti, C, Desiderio, M A (2003) Exp Cell Res 290:391-401
Takayama, H, LaRochelle, W. J, Sharp, R, Otsuka, T, Kriebel, P, Anver, M, Aaronson, S. A, Merlino, G. (1997) Proc Natl Acad Sci USA 94:701-706
Tikhomirov, O, Carpenter, G (2000) J Biol Chem 275:26625-31
Webb, C P, Hose, C D, Koochekpour, S, Jeffers, M, Oskarsson, M, Sausville, E, Monks, A, Vande
Woude, G F (2000) Cane Res 60:342-9
Whitesell, L, Cook, P (1996) Mol Endocrinol 10:705-12
Whitesell, L, Mimnaugh, E G, De Costa, B, Myers, C E, Neckers, L M. (1994) Proc Natl Acad Sci USA 91:8324-8328
Workman, P. (2003) Curr Cancer Drug Targets 3:297-300
Wright, C, Shulkind, M, Jones, K, Thompson, M. (1987) Tetrahedron Lett 28:6389-6390
Yamamoto, M, Sawaya, R, Mohanam, S, Bindal, A K, Bruner, J M, Oka, K, Rao, V H, Tomonaga, M, Nicolson, G L, Rao, J S (1994) Cane Res 54:3656-61
Yang, J, Yang, J M, Iannone, M, Shih, W J, Lin, Y, Hait, W N (2001) Canc Res 61:4010-6
Zhang, X, Bu, X Y, Zhen, H N, Fei, Z, Zhang, J N, Fu, L A (2000) J Clin Neurosci 7:116-9

All the references cited above are incorporated herein by reference in their entirety, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

1. A compound of 17-N-Aziridinyl-17-demethoxygeldanamycin or a pharmaceutically acceptable salt thereof.

2. (canceled)

3. (canceled)

4. (canceled)

5. (canceled)

6. (canceled)

7. (canceled)

8. (canceled)

9. (canceled)

10. A pharmaceutical compositions comprising
(a) the compound of claim 1; and
(b) a pharmaceutically acceptable carrier or excipient.

11. A method of inhibiting the HGF/SF-induced, Met receptor mediated biological activity of a Met-bearing tumor or cancer cell, comprising providing to said cell an effective amount of a compound of Formula I or Formula II 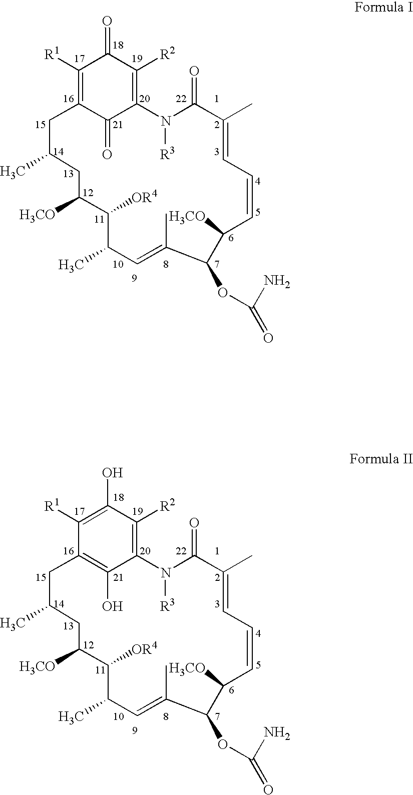
pharmaceutically acceptable salt thereof;
which compound has an IC₅₀ of more than about 10⁻¹⁰ M for inhibition of said biological activity, wherein
R¹ is a lower alkyl, alkenyl or alkynyl; a substituted lower alkyl, alkenyl or alkynyl; a lower alkoxy, alkenoxy or alkynoxy; a straight or branched alkylamine, alkenyl amine or alkynyl amine; or a 3-6 member heterocyclic group that is optionally substituted;
R² is H, a lower alkyl, alkenyl or alkynyl, a substituted lower alkyl, alkenyl or alkynyl; a lower alkoxy, alkenoxy or alkynoxy; a straight or branched alkylamine, alkenyl amine or alkynyl amines; or a 3-6 member heterocyclic group that is optionally substituted;
R³ is H; a lower alkyl, alkenyl or alkynyl; a substituted lower alkyl, alkenyl or alkynyl; a lower alkoxy, alkenoxy or alkynoxy; a straight or branched alkylamine, alkenyl amine or alkynyl amine; or wherein the N is a member or a heterocycloalkyl, heterocylokenyl or heteroaryl ring that is optionally substituted;
R⁴ is H; a lower alkyl, alkenyl or alkynyl, a substituted lower alkyl, alkenyl or alkynyl, an wherein
the bonds linking positions C₂ and C₃, C₄ and C₅, and C₈ and C₉ are optionally single bonds.

12. The method of claim 11 wherein said biological activity is the induction of uPA activity in said cells.

13. The method of claim 11 wherein said biological activity is growth or scatter of said cells.

14. The method of claim 13 wherein said growth of said cells is in vitro.

15. The method of claim 13 wherein said growth of said cells is in vivo.

16. The method of claim 11 wherein said biological activity is invasion of said cells.

17. The method of claim 16 wherein said invasion is in vitro.

18. The method of claim 16 wherein said invasion is in vivo.

19. The method of claim 16 wherein said invasion results in tumor metastasis.

20. A method of inhibiting in a subject metastasis of Met-bearing tumor or cancer cells that is induced by HGF/SF, comprising providing to said subject an effective amount of a compound of Formula I or Formula II 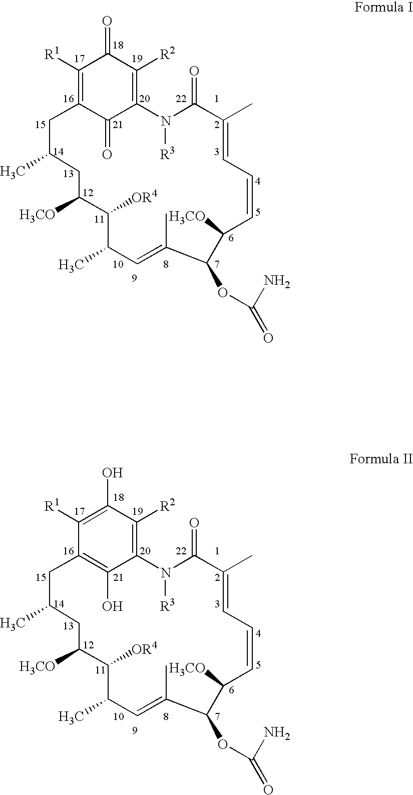
pharmaceutically acceptable salt thereof;
which compound has an IC₅₀ of more than about 10⁻¹⁰ M for inhibition of tumor cell invasion when measured in an assay in vitro,
wherein R¹ is a lower alkyl, alkenyl or alkynyl; a substituted lower alkyl, alkenyl or alkynyl; a lower alkoxy, alkenoxy or alkynoxy; a straight or branched alkylamine, alkenyl amine or alkynyl amine; or a 3-6 member heterocyclic group that is optionally substituted;
R² is H, a lower alkyl, alkenyl or alkynyl, a substituted lower alkyl, alkenyl or allynyl; a lower alkoxy, alkenoxy or alkynoxy; a straight or branched alkylamine, alkenyl amine or alkynyl amines; or a 3-6 member heterocyclic group that is optionally substituted;
R³ is H; a lower alkyl, alkenyl or alkynyl; a substituted lower alkyl, alkenyl or alkynyl; a lower alkoxy, alkenoxy or alkynoxy; a straight or branched alkylamine, alkenyl amine or alkynyl amine; or wherein the N is a member of a heterocycloalkyl, heterocycloalkenyl or heteroaryl ring that is optionally substituted;
R⁴ is H; a lower alkyl, alkenyl or alkynyl, a substituted lower alkyl, alkenyl or alkynyl, and wherein
the bonds linking positions C₂ and C₃, C₄ and C₅, and C₈ and C₉ are optionally single bonds.

21. A method of inhibiting in a subject metastasis of Met-bearing tumor or cancer cells that is induced by HGF/SF, comprising providing to said subject an effective amount of a pharmaceutical composition according to claim 10 which composition comprises a chemical compound that has an IC₅₀ of more than about 10⁻¹⁰ for inhibition of tumor cell invasion when measured in an assay in vitro.

22. The method of claim 11 wherein said inhibition results in measurable regression of a tumor caused by said cells or measurable attenuation of tumor growth in said subject.

23. A method of protecting against growth or metastasis of a Met-positive tumor in a susceptible subject, comprising administering to said subject who is either
(a) at risk for development of said tumor, or
(b) in the case of an already treated subject, at risk for recurrence of said tumor, an effective amount of a compound of Formula I or Formula II 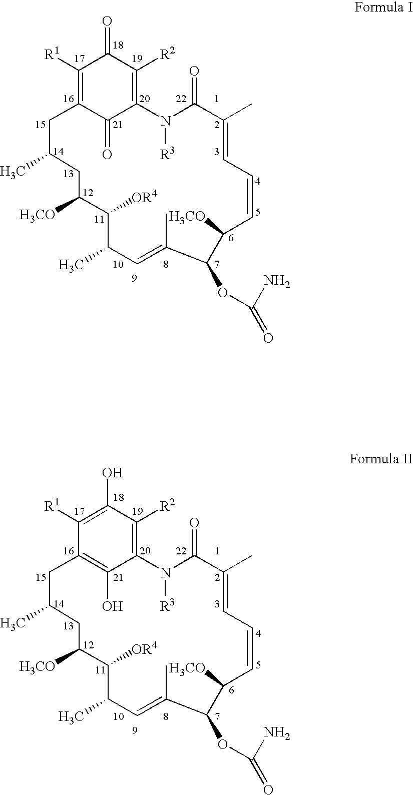
pharmaceutically acceptable salt thereof;
which compound has an IC₅₀ of more than about 10⁻¹⁰ M for inhibiting Met activation of uPA in cancer cells,
wherein R¹ is a lower alkyl, alkenyl or alkynyl; a substituted lower alkyl, alkenyl or alkynyl; a lower alkoxy, alkenoxy or alkynoxy; a straight or branched alkylamine, alkenyl amine or alkynyl amine; or a 3-6 member heterocyclic group that is optionally substituted;
R² is H, a lower alkyl, alkenyl or alkynyl, a substituted lower alkyl, alkenyl or allynyl; a lower alkoxy, alkenoxy or alkynoxy; a straight or branched alkylamine, alkenyl amine or alkynyl amines; or a 3-6 member heterocyclic group that is optionally substituted;
R³ is H; a lower alkyl, alkenyl or alkynyl; a substituted lower alkyl, alkenyl amine or alkynyl; a lower alkoxy, alkenoxy or alkynoxy; a straight or branched alkylamine, alkenyl amine or alkynyl amine; or wherein the N is a member of a heterocycloalkyl, heterocylokenyl or heteroaryl ring that is optionally substituted;
R⁴ is H; a lower alkyl, alkenyl or alkynyl, a substituted lower alkyl, alkenyl or alkynyl, and wherein
the bonds linking positions C₂ and C₃, C₄ and C₅, and C₈ and C₉ are optionally single bonds.

24. The method of claim 23 wherein the subject is a human.

25. A method of inducing an antitumor or anticancer response in a mammal having an HGF-responsive Met-expressing tumor, comprising administering to said mammal an effective amount of a compound of Formula I or Formula II 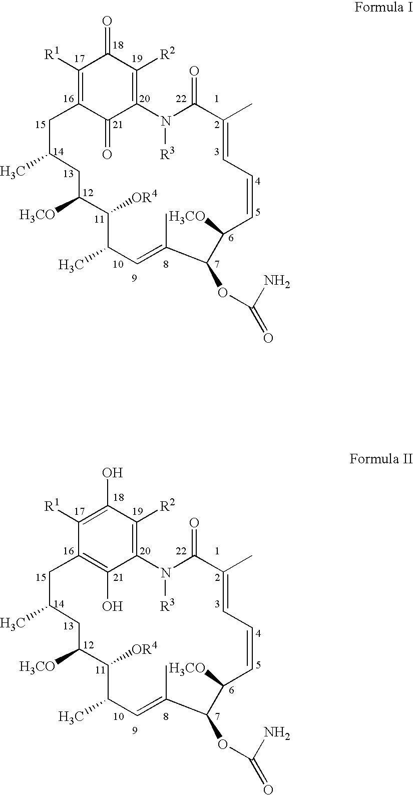
pharmaceutically acceptable salt thereof, at a concentration of more than about 10⁻¹⁰ M;
wherein R¹ is a lower alkyl, alkenyl or alkynyl; a substituted lower alkyl, alkenyl or alkynyl; a lower alkoxy, alkenoxy; a straight or branched alkylamine, alkenyl amine or alkynyl amine; or a 3-6 member heterocyclic group that is optionally substituted;
R² is H, a lower alkyl, alkenyl or alkynyl, a substituted lower alkyl, alkenyl or allynyl; a lower alkoxy, alkenoxy or alkynoxy; a straight or branched alkylamine, alkenyl amine or alkynyl amine; or a 3-6 member heterocyclic group that is optionally substituted;
R³ is H; a lower alkyl, alkenyl or alkynyl; a substituted lower alkyl, alkenyl or alkynyl; a lower alkoxy, alkenoxy or alkynoxy; a straight or branched alkylamine, alkenyl amine or alkynyl amine; or wherein the N is a member of a heterocycloalkyl, heterocylokenyl or heteroaryl ring that is optionally substituted;
R⁴ is H; a lower alkyl, alkenyl or alkynyl, a substituted lower alkyl, alkenyl or alkynyl, and wherein
the bonds linking positions C₂ and C₃, C₄ and C₅, and C₈ and C₉ are optionally single bonds,
thereby inducing an antitumor or anticancer response which is
(a) a partial response characterized by
(i) at least a 50% decrease in the sum of the products of maximal perpendicular diameters of all measurable lesions;
(ii) no evidence of new lesions, and
(iii) no progression of any preexisting lesions, or
(b) a complete response characterized by the disappearance of all evidence of tumor or cancer disease for at least one month.

26. The method of claim 25 wherein said antitumor or anticancer response is a partial antitumor or anticancer response.

27. The method of claim 25 wherein the mammal is a human.

28. A compound according to claim 1 which is detectably labeled with a halogen radionuclide.

29. The compound of claim 28 wherein the radionuclide is bonded to the R¹ group.

30. The compound of claim 28 wherein the radionuclide is selected from the group consisting of ¹⁸F, ⁷⁶Br, ¹²³I, ¹²⁴I, and ¹³¹I.

31. A method of imaging a tumor in a subject comprising administering an effective amount of a labeled compound according to claim 28, and imaging the detectable label with an imaging means.

32. The method of claim 11 wherein the compound is a benzoquinone of Formula I.

33. The method of claim 11 wherein the compound is a hydroquinone of Formula II.

34. The method of claim 11 wherein R¹ is a 3-6 member heterocyclic ring in which the heteroatom is N.

35. The method of claim 11 wherein each of R², R³ and R⁴ of the compound is H.

36. The method of claim 11 wherein the compound is selected from the group consisting of:
(a) 17-(2-Fluoroethyl)amino-17-demethoxygeldanamycin;
(b) 17-Allylamino-17-demethoxygeldanamycin;
(c) 17-N-Aziridinyl-17-demethoxygeldanamycin;
(d) 17-Amino-17-demethoxygeldanamycin;
(e) 17-N-Azetidinyl-17-demethoxygeldanamycin;
(f) 17-(2-Dimethylaminoethyl)amino-17-demethoxygeldanamycin;
(g) 17-(2-Chloroethyl)amino-17-demethoxygeldanamycin; and
(h) Dihydrogeldanamycin.
